SEQUENCE LISTING (84 sequences)

SEQ ID NO: 1 - DNA, 53 bp, synthetic construct
  misc_feature 1..53: note = Description of Artificial Sequence: Synthetic oligonucleotide
  source 1..53: mol_type = other DNA
caccacagccgaatctcgcaatgaatctcgagattcattgcgagattcggctg

SEQ ID NO: 2 - RNA, 25 nt, synthetic construct
  misc_feature 1..25: note = Description of Artificial Sequence: Synthetic oligonucleotide
  source 1..25: mol_type = other RNA
acatggacgagatggatacaggtga

SEQ ID NO: 3 - DNA, 21 bp, synthetic construct
  misc_feature 1..21: note = Description of Artificial Sequence: Synthetic primer
  source 1..21: mol_type = other DNA
tcagtcaacgggggacataaa

SEQ ID NO: 4 - DNA, 21 bp, synthetic construct
  misc_feature 1..21: note = Description of Artificial Sequence: Synthetic primer
  source 1..21: mol_type = other DNA
ggggctgtactgcttaaccag

SEQ ID NO: 5 - DNA, 21 bp, synthetic construct
  misc_feature 1..21: note = Description of Artificial Sequence: Synthetic primer
  source 1..21: mol_type = other DNA
catggcggaaaaagatcctcc

SEQ ID NO: 6 - DNA, 21 bp, synthetic construct
  misc_feature 1..21: note = Description of Artificial Sequence: Synthetic primer
  source 1..21: mol_type = other DNA
gtcggtcacgtcataggactg

SEQ ID NO: 7 - DNA, 19 bp, synthetic construct
  misc_feature 1..19: note = Description of Artificial Sequence: Synthetic primer
  source 1..19: mol_type = other DNA
ctcccgtggcttctagtgc

SEQ ID NO: 8 - DNA, 22 bp, synthetic construct
  misc_feature 1..22: note = Description of Artificial Sequence: Synthetic primer
  source 1..22: mol_type = other DNA
gccttagtttggacaggatctg

SEQ ID NO: 9 - DNA, 24 bp, synthetic construct
  misc_feature 1..24: note = Description of Artificial Sequence: Synthetic primer
  source 1..24: mol_type = other DNA
atgctcccagggctgttttcccat

SEQ ID NO: 10 - DNA, 25 bp, synthetic construct
  misc_feature 1..25: note = Description of Artificial Sequence: Synthetic primer
  source 1..25: mol_type = other DNA
gtggtgccagatcttttccatgtcg

SEQ ID NO: 11 - DNA, 19 bp, synthetic construct
  misc_feature 1..19: note = Description of Artificial Sequence: Synthetic primer
  source 1..19: mol_type = other DNA
tttctctgcctctgccaac

SEQ ID NO: 12 - DNA, 22 bp, synthetic construct
  misc_feature 1..22: note = Description of Artificial Sequence: Synthetic primer
  source 1..22: mol_type = other DNA
tctcattgatcacctgtccatc

SEQ ID NO: 13 - DNA, 20 bp, synthetic construct
  misc_feature 1..20: note = Description of Artificial Sequence: Synthetic primer
  source 1..20: mol_type = other DNA
ctaaaggatgagatggcccg

SEQ ID NO: 14 - DNA, 22 bp, synthetic construct
  misc_feature 1..22: note = Description of Artificial Sequence: Synthetic primer
  source 1..22: mol_type = other DNA
gaaggtctggataggaaggttg

SEQ ID NO: 15 - DNA, 19 bp, synthetic construct
  misc_feature 1..19: note = Description of Artificial Sequence: Synthetic primer
  source 1..19: mol_type = other DNA
gctcctcttaggggccact

SEQ ID NO: 16 - DNA, 19 bp, synthetic construct
  misc_feature 1..19: note = Description of Artificial Sequence: Synthetic primer
  source 1..19: mol_type = other DNA
ccacgtctcaccattgggg

SEQ ID NO: 17 - DNA, 21 bp, synthetic construct
  misc_feature 1..21: note = Description of Artificial Sequence: Synthetic primer
  source 1..21: mol_type = other DNA
gtaacttcgtgcctagcaaca

SEQ ID NO: 18 - DNA, 23 bp, synthetic construct
  misc_feature 1..23: note = Description of Artificial Sequence: Synthetic primer
  source 1..23: mol_type = other DNA
cctttgtcagaatactgagcagc

SEQ ID NO: 19 - DNA, 23 bp, synthetic construct
  misc_feature 1..23: note = Description of Artificial Sequence: Synthetic primer
  source 1..23: mol_type = other DNA
ctgtaacatggaaactggggaaa

SEQ ID NO: 20 - DNA, 23 bp, synthetic construct
  misc_feature 1..23: note = Description of Artificial Sequence: Synthetic primer
  source 1..23: mol_type = other DNA
ccatagctgaactgaaaaccacc

SEQ ID NO: 21 - DNA, 21 bp, synthetic construct
  misc_feature 1..21: note = Description of Artificial Sequence: Synthetic primer
  source 1..21: mol_type = other DNA
accacaatacctacatgcacc

SEQ ID NO: 22 - DNA, 21 bp, synthetic construct
  misc_feature 1..21: note = Description of Artificial Sequence: Synthetic primer
  source 1..21: mol_type = other DNA
aagcaggcgaggaaaagatag

SEQ ID NO: 23 - DNA, 22 bp, synthetic construct
  misc_feature 1..22: note = Description of Artificial Sequence: Synthetic primer
  source 1..22: mol_type = other DNA
cttctgtctactgaacttcggg

SEQ ID NO: 24 - DNA, 22 bp, synthetic construct
  misc_feature 1..22: note = Description of Artificial Sequence: Synthetic primer
  source 1..22: mol_type = other DNA
caggcttgtcactcgaattttg

SEQ ID NO: 25 - DNA, 23 bp, synthetic construct
  misc_feature 1..23: note = Description of Artificial Sequence: Synthetic primer
  source 1..23: mol_type = other DNA
ttaaaaacctggatcggaaccaa

SEQ ID NO: 26 - DNA, 23 bp, synthetic construct
  misc_feature 1..23: note = Description of Artificial Sequence: Synthetic primer
  source 1..23: mol_type = other DNA
gcattagcttcagatttacgggt

SEQ ID NO: 27 - DNA, 21 bp, synthetic construct
  misc_feature 1..21: note = Description of Artificial Sequence: Synthetic primer
  source 1..21: mol_type = other DNA
ctcttgcctacaagcagttca

SEQ ID NO: 28 - DNA, 19 bp, synthetic construct
  misc_feature 1..19: note = Description of Artificial Sequence: Synthetic primer
  source 1..19: mol_type = other DNA
ccgtgttctcctcgtcctt

SEQ ID NO: 29 - DNA, 20 bp, synthetic construct
  misc_feature 1..20: note = Description of Artificial Sequence: Synthetic primer
  source 1..20: mol_type = other DNA
caacgcctactctcccagac

SEQ ID NO: 30 - DNA, 22 bp, synthetic construct
  misc_feature 1..22: note = Description of Artificial Sequence: Synthetic primer
  source 1..22: mol_type = other DNA
gagccttgatgtactgtaccac

SEQ ID NO: 31 - DNA, 19 bp, synthetic construct
  misc_feature 1..19: note = Description of Artificial Sequence: Synthetic primer
  source 1..19: mol_type = other DNA
cacagctctacggcgactg

SEQ ID NO: 32 - DNA, 23 bp, synthetic construct
  misc_feature 1..23: note = Description of Artificial Sequence: Synthetic primer
  source 1..23: mol_type = other DNA
ctgcatagtgattgcgtttcttc

SEQ ID NO: 33 - DNA, 25 bp, synthetic construct
  misc_feature 1..25: note = Description of Artificial Sequence: Synthetic primer
  source 1..25: mol_type = other DNA
ctgacccatctcagaagcagaatct

SEQ ID NO: 34 - DNA, 25 bp, synthetic construct
  misc_feature 1..25: note = Description of Artificial Sequence: Synthetic primer
  source 1..25: mol_type = other DNA
tccatgtggtcatggctttcattgg

SEQ ID NO: 35 - DNA, 22 bp, synthetic construct
  misc_feature 1..22: note = Description of Artificial Sequence: Synthetic primer
  source 1..22: mol_type = other DNA
ctcaaagacctatagtgctggc

SEQ ID NO: 36 - DNA, 20 bp, synthetic construct
  misc_feature 1..20: note = Description of Artificial Sequence: Synthetic primer
  source 1..20: mol_type = other DNA
caaagtgacggctctggtag

SEQ ID NO: 37 - DNA, 20 bp, synthetic construct
  misc_feature 1..20: note = Description of Artificial Sequence: Synthetic primer
  source 1..20: mol_type = other DNA
gcacaccaggcagtagcttt

SEQ ID NO: 38 - DNA, 23 bp, synthetic construct
  misc_feature 1..23: note = Description of Artificial Sequence: Synthetic primer
  source 1..23: mol_type = other DNA
caggagttgattccagacaggta

SEQ ID NO: 39 - DNA, 20 bp, synthetic construct
  misc_feature 1..20: note = Description of Artificial Sequence: Synthetic primer
  source 1..20: mol_type = other DNA
tcgctgatgcactgcctatg

SEQ ID NO: 40 - DNA, 21 bp, synthetic construct
  misc_feature 1..21: note = Description of Artificial Sequence: Synthetic primer
  source 1..21: mol_type = other DNA
gagaggtccacagagctgatt

SEQ ID NO: 41 - DNA, 20 bp, synthetic construct
  misc_feature 1..20: note = Description of Artificial Sequence: Synthetic primer
  source 1..20: mol_type = other DNA
ctgacctgaaagccgagaag

SEQ ID NO: 42 - DNA, 20 bp, synthetic construct
  misc_feature 1..20: note = Description of Artificial Sequence: Synthetic primer
  source 1..20: mol_type = other DNA
agaaggtgctaacgaacagg

SEQ ID NO: 43 - DNA, 21 bp, synthetic construct
  misc_feature 1..21: note = Description of Artificial Sequence: Synthetic primer
  source 1..21: mol_type = other DNA
aagtcccagaaatcgcctatg

SEQ ID NO: 44 - DNA, 21 bp, synthetic construct
  misc_feature 1..21: note = Description of Artificial Sequence: Synthetic primer
  source 1..21: mol_type = other DNA
ggtatggtttcacgactggag

SEQ ID NO: 45 - DNA, 21 bp, synthetic construct
  misc_feature 1..21: note = Description of Artificial Sequence: Synthetic primer
  source 1..21: mol_type = other DNA
atgggcggaatggtctctttc

SEQ ID NO: 46 - DNA, 21 bp, synthetic construct
  misc_feature 1..21: note = Description of Artificial Sequence: Synthetic primer
  source 1..21: mol_type = other DNA
tggggaccttgtcttcatcat

SEQ ID NO: 47 - DNA, 21 bp, synthetic construct
  misc_feature 1..21: note = Description of Artificial Sequence: Synthetic primer
  source 1..21: mol_type = other DNA
atgcggtggaacactttctgg

SEQ ID NO: 48 - DNA, 21 bp, synthetic construct
  misc_feature 1..21: note = Description of Artificial Sequence: Synthetic primer
  source 1..21: mol_type = other DNA
gcacgtcaactctacactggt

SEQ ID NO: 49 - DNA, 21 bp, synthetic construct
  misc_feature 1..21: note = Description of Artificial Sequence: Synthetic primer
  source 1..21: mol_type = other DNA
aacctgctccaaaagtgaacc

SEQ ID NO: 50 - DNA, 22 bp, synthetic construct
  misc_feature 1..22: note = Description of Artificial Sequence: Synthetic primer
  source 1..22: mol_type = other DNA
cccagggtaatatggaagaggc

SEQ ID NO: 51 - DNA, 20 bp, synthetic construct
  misc_feature 1..20: note = Description of Artificial Sequence: Synthetic primer
  source 1..20: mol_type = other DNA
caatcattgacagcgagggc

SEQ ID NO: 52 - DNA, 21 bp, synthetic construct
  misc_feature 1..21: note = Description of Artificial Sequence: Synthetic primer
  source 1..21: mol_type = other DNA
ggtgtagctcggttctggtag

SEQ ID NO: 53 - DNA, 21 bp, synthetic construct
  misc_feature 1..21: note = Description of Artificial Sequence: Synthetic primer
  source 1..21: mol_type = other DNA
ggtgtagctcggttctggtag

SEQ ID NO: 54 - DNA, 20 bp, synthetic construct
  misc_feature 1..20: note = Description of Artificial Sequence: Synthetic primer
  source 1..20: mol_type = other DNA
tcacctgggactccatttgc

SEQ ID NO: 55 - DNA, 19 bp, synthetic construct
  misc_feature 1..19: note = Description of Artificial Sequence: Synthetic primer
  source 1..19: mol_type = other DNA
tcattgcgagattcggctg

SEQ ID NO: 56 - DNA, 19 bp, synthetic construct
  misc_feature 1..19: note = Description of Artificial Sequence: Synthetic primer
  source 1..19: mol_type = other DNA
gatgaatccgtcctcggtg

SEQ ID NO: 57 - DNA, 25 bp, synthetic construct
  misc_feature 1..25: note = Description of Artificial Sequence: Synthetic primer
  source 1..25: mol_type = other DNA
gaggcaagttgaaaggtcagaggca

SEQ ID NO: 58 - DNA, 25 bp, synthetic construct
  misc_feature 1..25: note = Description of Artificial Sequence: Synthetic primer
  source 1..25: mol_type = other DNA
gctgcaccacgttctgcctttgtac

SEQ ID NO: 59 - DNA, 25 bp, synthetic construct
  misc_feature 1..25: note = Description of Artificial Sequence: Synthetic primer
  source 1..25: mol_type = other DNA
ggcaatgacgtccttagctgtttag

SEQ ID NO: 60 - DNA, 25 bp, synthetic construct
  misc_feature 1..25: note = Description of Artificial Sequence: Synthetic primer
  source 1..25: mol_type = other DNA
aggcagcttggtccaggaagtgatt

SEQ ID NO: 61 - DNA, 22 bp, synthetic construct
  misc_feature 1..22: note = Description of Artificial Sequence: Synthetic primer
  source 1..22: mol_type = other DNA
acctcttcaactctgtcatgaa

SEQ ID NO: 62 - DNA, 22 bp, synthetic construct
  misc_feature 1..22: note = Description of Artificial Sequence: Synthetic primer
  source 1..22: mol_type = other DNA
cgccctttctaacctggctgta

SEQ ID NO: 63 - DNA, 25 bp, synthetic construct
  misc_feature 1..25: note = Description of Artificial Sequence: Synthetic primer
  source 1..25: mol_type = other DNA
tgccaccgttcatcattttcctgct

SEQ ID NO: 64 - DNA, 25 bp, synthetic construct
  misc_feature 1..25: note = Description of Artificial Sequence: Synthetic primer
  source 1..25: mol_type = other DNA
tccccgagtccccagaaagatgaat

SEQ ID NO: 65 - DNA, 25 bp, synthetic construct
  misc_feature 1..25: note = Description of Artificial Sequence: Synthetic primer
  source 1..25: mol_type = other DNA
ccagctcatggcggaaaaagatcct

SEQ ID NO: 66 - DNA, 25 bp, synthetic construct
  misc_feature 1..25: note = Description of Artificial Sequence: Synthetic primer
  source 1..25: mol_type = other DNA
accccaggaaggtgatgaatcagcc

SEQ ID NO: 67 - DNA, 25 bp, synthetic construct
  misc_feature 1..25: note = Description of Artificial Sequence: Synthetic primer
  source 1..25: mol_type = other DNA
gggccttgcggaccaagtgatgagg

SEQ ID NO: 68 - DNA, 25 bp, synthetic construct
  misc_feature 1..25: note = Description of Artificial Sequence: Synthetic primer
  source 1..25: mol_type = other DNA
gcccttgactgtttactaatagata

SEQ ID NO: 69 - DNA, 25 bp, synthetic construct
  misc_feature 1..25: note = Description of Artificial Sequence: Synthetic primer
  source 1..25: mol_type = other DNA
ccaaatccatcagatgaaaccattt

SEQ ID NO: 70 - DNA, 22 bp, synthetic construct
  misc_feature 1..22: note = Description of Artificial Sequence: Synthetic primer
  source 1..22: mol_type = other DNA
gcctgcatttctgtggcagata

SEQ ID NO: 71 - DNA, 24 bp, synthetic construct
  misc_feature 1..24: note = Description of Artificial Sequence: Synthetic primer
  source 1..24: mol_type = other DNA
gccatgagcacagatatgagatct

SEQ ID NO: 72 - DNA, 21 bp, synthetic construct
  misc_feature 1..21: note = Description of Artificial Sequence: Synthetic primer
  source 1..21: mol_type = other DNA
tcccagccaaatctcgtgatg

SEQ ID NO: 73 - DNA, 32 bp, Mus sp.
  source 1..32: mol_type = unassigned DNA
ggccgggccgggctgggcgccgaggaatgcag

SEQ ID NO: 74 - DNA, 32 bp, Homo sapiens
  source 1..32: mol_type = unassigned DNA
ggccgggccgggccgggcgccgaggaatgcag

SEQ ID NO: 75 - DNA, 32 bp, Pan troglodytes; identical to SEQ ID NO: 74
  source 1..32: mol_type = unassigned DNA

SEQ ID NO: 76 - DNA, 32 bp, Tursiops sp.; identical to SEQ ID NO: 74
  source 1..32: mol_type = unassigned DNA

SEQ ID NO: 77 - DNA, 32 bp, Sus sp.; identical to SEQ ID NO: 74
  source 1..32: mol_type = unassigned DNA

SEQ ID NO: 78 - DNA, 32 bp, Oryctolagus cuniculus; identical to SEQ ID NO: 74
  source 1..32: mol_type = unassigned DNA

SEQ ID NO: 79 - DNA, 32 bp, Rattus sp.; identical to SEQ ID NO: 73
  source 1..32: mol_type = unassigned DNA

SEQ ID NO: 80 - skipped

SEQ ID NO: 81 - DNA, 20 bp, synthetic construct
  misc_feature 1..20: note = Description of Artificial Sequence: Synthetic primer
  source 1..20: mol_type = other DNA
agcgcattgggcatactcat

SEQ ID NO: 82 - RNA, 25 nt, synthetic construct
  misc_feature 1..25: note = Description of Artificial Sequence: Synthetic oligonucleotide
  source 1..25: mol_type = other RNA
tgcggacaatcatacagaaccagca

SEQ ID NO: 83 - RNA, 22 nt, synthetic construct
  misc_feature 1..22: note = Description of Artificial Sequence: Synthetic oligonucleotide
  source 1..22: mol_type = other RNA
accaccttcagtgatgtgctta

SEQ ID NO: 84 - RNA, 25 nt, synthetic construct
  misc_feature 1..25: note = Description of Artificial Sequence: Synthetic oligonucleotide
  source 1..25: mol_type = other RNA
ggatactagttgtgaaatggaaaga

(19) United States
(12) Patent Application Publication
(10) Pub. No.: US 2024/0043839 A1
(43) Pub. Date: Feb. 8, 2024

(54) IHH AS A BIOMARKER AND THERAPEUTIC TARGET FOR NONALCOHOLIC STEATOHEPATITIS (NASH)

(71) Applicant: The Trustees of Columbia University in the City of New York, New York, NY (US)

(72) Inventors: Ira TABAS, New York, NY (US); Xiaobo WANG, New York, NY (US)

(21) Appl. No.: 18/299,799

(22) Filed: Apr. 13, 2023

Related U.S. Application Data
(63) Continuation of application No. PCT/US21/54692, filed on Oct. 13, 2021.
(60) Provisional application No. 63/091,618, filed on Oct. 14, 2020.

(51) Int. Cl. C12N 15/113; G01N 33/68; A61P 1/16

(52) U.S. Cl. CPC C12N 15/113; G01N 33/6893; A61P 1/16; C12N 2310/14; G01N 2800/085

(57) ABSTRACT

The present invention relates to methods and compositions for using plasma Indian hedgehog (IHH) as a biomarker for diagnosing, and as a therapeutic target for treating, liver conditions including non-alcoholic steatohepatitis (NASH).

### CROSS-REFERENCE TO RELATED APPLICATIONS

This present application is a continuation application of PCT Application No. PCT/US2021/054692, which claims priority to U.S. Provisional Patent Application No. 63/091,618 filed on Oct. 14, 2020, the content of each of which is incorporated herein by reference in its entirety.

### GOVERNMENT SUPPORT

This invention was made with government support under DK116620 awarded by the National Institutes of Health (NIH). The government has certain rights in the invention.

### SEQUENCE LISTING

The instant application contains a Sequence Listing which has been submitted electronically in XML format and is hereby incorporated by reference in its entirety. Said XML copy, created on Jun. 20, 2023, is named 01001_009043-US1_SL.xml and is 106,309 bytes in size.

### FIELD OF THE INVENTION

The present invention relates to methods of diagnosing liver conditions including NASH.

### BACKGROUND

Nonalcoholic steatohepatitis (NASH) has emerged as the leading cause of chronic liver disease worldwide. However, there is a dearth of treatment options, which is due in large part to a poor understanding of how benign steatosis progresses to NASH. NASH is a common and serious complication of obesity and type 2 diabetes, but many gaps remain in our understanding of its pathophysiology, leading to a lack of treatment options (White et al., 2012). NASH most likely develops as a result of multiple hits (Day and James, 1998), including steatosis, driven by hyperinsulinemia and elevated free fatty acid delivery to the liver, in combination with insults that promote inflammation, fibrosis, and hepatocyte death (Singh et al., 2015). However, the molecular mechanisms corresponding to these pathogenic processes and their integration are poorly understood. The incomplete understanding of NASH can be explained in part by the paucity of animal models that combine steatosis, obesity/insulin resistance, and key features of NASH, such as inflammation and fibrosis, as well as by insufficient integration of experimental results with human NASH findings.

There is a great need for NASH biomarkers and for new therapeutic targets for treating or preventing NASH and related conditions.

### SUMMARY

The present disclosure provides for a method for diagnosing a liver condition (or disease) or susceptibility to a liver condition (or disease) in a subject, where the liver condition may be a fatty liver disease, a non-alcoholic fatty liver disease, adiposity, and combinations thereof. The method may comprise: (a) obtaining a sample from the subject; (b) determining a level of Indian hedgehog (IHH) in the sample; (c) comparing the level of IHH in the sample with a level of IHH in a control sample; and (d) diagnosing that the subject has the liver condition or diagnosing the subject as being susceptible to the liver condition, if the level of IHH in the sample increases by at least 25% (or at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, about 20% to about 90%, about 50% to about 100%, at least 1 fold, at least 1.5 folds, at least 2 folds, at least 2.5 folds, or at least 3 folds) compared to its level in the control sample.

The present disclosure also provides for a method of treating a subject with a liver condition or susceptible to a liver condition, where the liver condition may be a fatty liver disease, a non-alcoholic fatty liver disease, adiposity, and combinations thereof. The method may comprise: (a) obtaining a sample from the subject; (b) determining a level of IHH in the sample; (c) comparing the level of IHH in the sample with a level of IHH in a control sample; and (d) treating the subject, if the level of IHH in the sample increases by at least 25% (or at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, about 20% to about 90%, about 50% to about 100%, at least 1 fold, at least 1.5 folds, at least 2 folds, at least 2.5 folds, or at least 3 folds) compared to its level in the control sample.

The non-alcoholic fatty liver disease may be non-alcoholic steatosis hepatis or non-alcoholic steatohepatitis (NASH).

The fatty liver disease may be steatosis hepatis or steatohepatitis.

The sample may be a plasma, serum or blood sample.

The level of IHH may be determined by enzyme-linked immunosorbent assay (ELISA), and/or mass spectrometry (MS).

In step (d), a therapeutically effective amount of an inhibitor of TAZ may be administered to the subject. The inhibitor of TAZ may be a protein, a nucleic acid, or combinations thereof. The nucleic acid may be an antisense oligonucleotide, a small interfering RNA (siRNA), a short hairpin RNA (shRNA), and combinations thereof. The nucleic acid may comprise a nucleic acid sequence comprising one or more of the following: SEQ ID NO:1, SEQ ID NO:2, SEQ ID NO:55-SEQ ID NO:72, and SEQ ID NO:81.

In step (d), a therapeutically effective amount of one or more of the following may be administered to the subject: an inhibitor of IHH, an inhibitor of YAP, an inhibitor of TEAD1, and inhibitor of TEAD2, an inhibitor of TEAD3, and an inhibitor of TEAD4.

In step (d), a therapeutically effective amount of one or more of the following may be administered to the subject: a therapeutic agent for treatment of steatosis hepatis, a therapeutic agent for treatment of steatohepatitis, a therapeutic agent for treatment of non-alcoholic fatty liver disease, a therapeutic agent for treatment of non-alcoholic steatohepatitis, a therapeutic agent for treatment of adiposity, and combinations thereof.

In step (d), a therapeutically effective amount of one or more of the following may be administered to the subject: an antidiabetic drug, and an insulin sensitizer.

In step (d), a therapeutically effective amount of one or more of the following may be administered to the subject: rosiglitazone; pioglitazone; losartan; simtuzumab; GR-MD-02; and obeticholic acid (OCA).

The control sample may be from a healthy subject or a plurality of healthy subjects.

The subject may be human.

The present disclosure provides for a kit comprising: antibodies or fragments thereof that specifically bind to IHH in a sample (e.g., a plasma or serum sample) from a subject; and instructions for measuring a level of IHH for diagnosing a liver condition in the subject or assessing the subject's risk of a liver condition.

In certain embodiments, the subject's existing therapy is modified or maintained.

In certain embodiments, an inhibitor of TAZ is administered to at least one hepatocyte of said patient.

In additional embodiments, the liver condition or disease is associated with imbalanced liver lipid metabolism and/or increased fat deposits.

In certain embodiments, the treatment may alleviate one or more symptoms associated with the liver condition. The one or more symptoms may be selected from the group consisting of hepatic inflammation, hepatocyte death, insulin resistance, weight gain, and liver fibrosis.

In certain embodiments, the liver fibrosis is associated with imbalanced liver lipid metabolism and/or increased fat deposits. In certain embodiments, the liver fibrosis is associated with non-alcoholic steatosis hepatis or non-alcoholic steatohepatitis (NASH). In certain embodiments, the liver fibrosis is associated with steatosis hepatis or steatohepatitis.

### BRIEF DESCRIPTION OF THE DRAWINGS

FIGS. 1A-1F are immunohistochemistry stains and blots illustrating that TAZ levels are increased in the livers of humans and mice with NASH. FIG. 1A is an immunohistochemistry staining showing TAZ immunofluorescence in normal, steatotic, and NASH human liver sections; DAPI counterstain for nuclei is shown in bottom panels. NASH-IgG refers to control for primary antibody. Bar, 100 μm. FIG. 1B is a graph showing the quantification of TAZ+ cells in normal, steatotic, and NASH human liver sections (*p<0.0001; mean±SEM; n=7 specimens/group). FIG. 1C shows immunoblots of TAZ in early NAFLD, normal, NASH, and steatotic human liver. For sake of comparison, samples #10-12 of NASH from the left blot were re-run with the steatosis samples in the right blot. GAPDH or β-actin were used as loading control. FIG. 1D shows an immunoblot of liver TAZ in mice fed chow or MCD diet, with β-actin as loading control. FIG. 1E shows an immunoblot of liver TAZ in mice fed chow or FPC diet, with β-actin as loading control. FIG. 1F is a graph showing the quantification of Taz mRNA in livers from mice fed chow or FPC diet (*p<0.0001; mean±SEM; n=6 mice/group.

FIGS. 2A-2Q are immunohistochemistry stains and blots showing that FPC-fed mice develop weight gain, insulin resistance, and features of NASH. The following parameters were measured in male C57BL/6J mice after 16 weeks on chow or FPC diet (*p<0.02, **p<0.0001; mean±SEM; n=6 mice/group): FIG. 2A-B are graphs showing body weight and liver:body weight ratio. FIG. 2C-E are graphs showing plasma fasting glucose, insulin, ALT, and AST. FIG. 2F is immunohistochemistry staining of liver sections with H&E (1st row; Bar, 100 μm), Masson's trichrome (Trichr) (2nd row; Bar, 100 μm), Sirius red (Sir red) (3rd row; Bar, 500 μm), and Oil Red O (ORO)/H&E (4th row; Bar, 100 μm). FIG. 2G-J are graphs quantifying lipid droplet area, liver inflammatory cell number, and aniline blue- and Sirius red-positive area. FIG. 2K is immunochemistry TUNEL staining and quantification; DAPI counterstain for nuclei is shown in bottom panels; Bar, 100 μm. FIG. 2L are graphs showing mRNA levels of Tnfa, Mcp1, and F4/80 (Adgre1). FIG. 2M is immunochemistry F4/80 immunofluorescence and graphic quantification; DAPI counterstain for nuclei is shown in bottom panels; Bar, 100 μm. FIG. 2N are graphs showing mRNA levels of Tgfb1 and Acta2 (α-SMA). FIG. 2O shows α-SMA immunohistochemistry; Bar, 200 μm. FIG. 2P shows filipin staining; Bar, 200 μm. FIG. 2Q is a graph showing liver cholesterol content.

FIGS. 3A-3G are immunohistochemistry stains and blots showing comparisons of liver parameters in mice fed the FPC vs. FF diet for 16 weeks. The following parameters were measured in male C57BL/6J mice after 16 weeks on chow, FPC, or FF diet (*p<0.05, **p<0.01, ***p<0.0001; mean±SEM; n=5 mice/group): FIG. 3A shows immunohistochemistry staining of liver sections for H&E (upper panels) and Masson's trichrome (Trichr) (lower panels); Bar, 100 μm. FIG. 3B-C are graphs showing quantification of hepatic inflammatory cells and aniline blue-positive area. FIG. 3D is a graph showing plasma ALT levels. FIG. 3E are graphs showing mRNA levels of Tnfa, Mcp1, F4/80 (Adgre1), Acta2 (α-SMA), and Tgfb1. FIG. 3F shows immunohistochemistry F4/80 immunofluorescence and graphic quantification; DAPI counterstain for nuclei is shown in bottom panels; Bar, 100 μm. FIG. 3G shows immunohistochemistry α-SMA immunofluorescence and graphic quantification; API counterstain for nuclei is shown in bottom panels; Bar, 100 μm.

FIGS. 4A-4J are immunohistochemistry stains and blots illustrating that TAZ silencing reduces liver inflammation, fibrosis, and cell death in FPC-fed mice. The following parameters were measured in male C57BL/6J mice treated with AAV8-shTaz or control vector and then fed the FPC diet for 16 weeks (*p<0.05, **p<0.01, ***p<0.0002, mean±SEM; n=10 mice/group): FIG. 4A shows an immunoblot of TAZ, with β-actin as loading control. FIG. 4B shows staining of liver sections for H&E (upper panels; Bar, 100 μm), Masson's trichrome (Trichr) (middle panels; Bar, 100 μm), and Sirius red (Sir red) (lower panels; Bar, 500 μm). FIG. 4C shows stains of hepatic inflammatory cells. FIG. 4D shows Aniline blue- and Sirius red-positive areas in the immunohistochemistry staining. FIG. 4E shows Plasma ALT. FIG. 4F shows TUNEL+ cells. FIG. 4G is a graph showing mRNA levels of Tnfa, Mcp1, and F4/80 (Adgre1). FIG. 4H is a graph showing mRNA levels of the indicated genes related to fibrosis. FIG. 4I shows immunohistochemistry F4/80 immunofluorescence staining and graphic quantification of the same data; DAPI counterstain for nuclei is shown in bottom panels; Bar, 100 μm. FIG. 4J shows immunohistochemistry α-SMA immunofluorescence and graphic quantification of the same data; DAPI counterstain for nuclei is shown in bottom panels; Bar, 100 μm.

FIGS. 5A-5H are immunohistochemistry stains and graphic quantifications illustrating that TAZ silencing reduces liver inflammation and fibrosis in FPC-fed Mc4r−/− hyperphagic mice. The following parameters were measured in male Mc4r−/− mice treated with AAV8-shTaz or control vector and then fed the FPC diet for 16 weeks (*p<0.05, **p<0.01, ***p<0.0001, mean±SEM; n=5 mice/group): FIG. 5A shows immunohistochemistry staining of liver sections for H&E (upper panels; Bar, 100 μm), Masson's trichrome (Trichr) (middle panels; Bar, 500 μm), and Sirius red (Sir red) (lower panels; Bar, 500 μm). FIG. 5B is a graph showing reduced hepatic inflammatory cells in shTaz treated mice compared with shControl mice. FIG. 5C is a graph showing Aniline blue- and Sirius red-positive staining areas in shTaz treated mice compared with shControl mice. FIG. 5D is a graph showing reduced Hydroxyproline content in shTaz treated mice compared with shControl mice. FIG. 5E is a graph showing reduced plasma ALT in shTaz treated mice compared with shControl mice. FIG. 5F are graphs showing mRNA levels of Tnfa, Mcp1, Tgfb1, Co11a1, and Timp1 in shTaz treated mice compared with shControl mice. FIG. 5G shows immunohistochemistry F4/80 immunofluorescence and quantification in shTaz treated mice compared with shControl mice; DAPI counterstain for nuclei is shown in bottom panels; Bar, 100 μm. FIG. 5H shows immunohistochemistry α-SMA immunofluorescence and quantification in shTaz treated mice compared with shControl mice; DAPI counterstain for nuclei is shown in bottom panels; Bar, 100 μm.

FIGS. 6A-6H are immunohistochemistry stains and graphic quantifications illustrating that TAZ silencing after the development of steatosis reduces liver inflammation and fibrosis in FPC-fed mice. FIG. 6A shows immunohistochemistry staining of liver sections for H&E (upper panels) and Masson's trichrome (Trichr) (lower panels) from C57BL/6J mice fed chow or FPC diet for 8 weeks; Bar, 100 μm. For FIG. 6B-H, the following parameters were measured in male C57BL/6J mice fed the FPC diet for 16 weeks, with AAV8-shTaz or control vector administered at the 8-week time point (*p<0.05; **p<0.01, ***p<0.0001, mean±SEM; n=5 mice/group): FIG. 6B shows immunohistochemistry staining of liver sections for H&E (upper panels; Bar, 100 μm), Masson's trichrome (Trichr) (middle panels; Bar, 100 μm), and Sirius red (Sir red) (lower panels; Bar, 500 μm). FIG. 6C is a graph showing reduced hepatic inflammatory cells in shTaz treated mice compared with sh control mice. FIG. 6D are graphs showing reduced Aniline blue- and Sirius red-positive areas in cells from shTaz treated mice compared with sh control mice. FIG. 6E is a graph showing reduced plasma ALT in shTaz treated mice compared with shControl mice. FIG. 6F are graphs showing reduced mRNA levels of Tnfa, Mcp1, Tgfb1, and Acta2 (α-SMA) in shTaz treated mice compared with sh control mice. FIG. 6G shows immunohistochemistry F4/80 immunofluorescence and graphic quantification of the same data in shTaz treated mice compared with sh control mice; DAPI counterstain for nuclei is shown in bottom panels; Bar, 100 μm. FIG. 6H shows immunohistochemistry α-SMA immunofluorescence and graphic quantification of the same data in shTaz treated mice compared with shControl mice; DAPI counterstain for nuclei is shown in bottom panels; Bar, 100 μm.

FIGS. 7A-7H are immunohistochemistry stains and graphic quantifications illustrating that TAZ Induces Ihh, and TAZ silencing lowers the expression of pro-fibrotic hedgehog pathway genes in the livers of FPC-fed mice. FIG. 7A is a sequence alignment showing the conserved TAZ/TEAD consensus sequence in intron 1 of the mouse Ihh gene. FIG. 7B are graphs showing quantitation of liver nuclear extracts from mice fed chow diet or FPC diet for 16 weeks with or without TAZ silencing were subjected to TAZ ChIP analysis using anti-TAZ or IgG control. The intronic region containing the TAZ/TEAD binding sequence, or a non-consensus sequence as control, was amplified by qPCR and normalized to the values obtained from input DNA (*p=0.03; mean±SEM; n=3). FIG. 7C is an immunoblot showing Ihh levels in normal human livers or those with steatosis or NASH. FIG. 7D is an immunoblot showing levels of Ihh in the livers of mice fed chow or FPC diet for 16 weeks. FIG. 7E is a graph showing relative expression of Ihh, Gli1, Gli2, and Opn mRNAs in the livers of mice fed chow or FPC diet for 16 weeks (*p<0.04, **p<0.0001; mean±SEM; n=6). FIG. 7F is a graph showing relative expression of Ihh, Gli1, Gli2, and Opn mRNAs in the livers of mice fed the FPC diet for 16 weeks with or without TAZ silencing (*p<0.05, **p<0.002; mean±SEM; n=10). FIG. 7G is an immunoblot of Ihh in the livers of mice fed the FPC diet for 16 weeks with or without TAZ silencing. FIG. 7H shows OPN immunohistochemistry and quantification in the livers of mice fed the FPC diet for 16 weeks with or without TAZ silencing (*p<0.0001; mean±SEM; n=10); Bar, 200 μm.

FIGS. 8A-8G are immunohistochemistry stains, blots, and graphic quantifications illustrating that TAZ-induced hepatocyte Ihh increases the expression of fibrosis-related genes in hepatic stellate cells. FIG. 8A is a graph showing expression of Taz and Ihh mRNA in control (Con) and TAZ-silenced AML12 hepatocytes (*p<0.0003; mean±SEM; n=3). FIG. 8B is an immunoblot of TAZ and Ihh in control and TAZ-silenced AML12 hepatocytes. FIG. 8C is a graph showing Ihh concentrations, assayed by ELISA, in the media of control and TAZ-silenced AML12 hepatocytes (*p<0.003; mean±SEM; n=3). FIG. 8D is a graph showing results from primary hepatic stellate cells (HSCs) that were incubated for 72 h with conditioned medium (CM) obtained from control (Con) or TAZ-silenced AML12 hepatocytes or with medium not exposed to cells (non-CM). The HSCs were then assayed for Opn, Timp1, and Col1a1 mRNA (upper panel; *p<0.05; mean±SEM; n=4) and the respective proteins by immunoblot (lower panel). FIG. 8E is a graph showing HSCs after 72 h incubation with non-CM or CM obtained from control (Con) or Ihh-silenced AML12 hepatocytes and then assayed for Opn, Timp1, and Col1a1 mRNA (*p<0.04; **p<0.0001, mean±SEM; n=4). FIG. 8F is a graph showing control (Con) or TAZ-silenced AML12 hepatocytes that were transduced with a plasmid encoding Ihh or control GFP. Aliquots of the four sets of conditioned medium were assayed for Ihh by ELISA (*p<0.002; mean±SEM; n=3). FIG. 8G is a graph showing HSCs that were incubated with conditioned media from the 4 sets of cells in (F) or with non-CM and then assayed for Opn, Timp1, and Col1a1 mRNA (*p<0.05; **p<0.004, ***p<0.0004, mean±SEM; n=4). Note that bars 2 and 3 for Opn and Col1a1 are significantly different at p<0.05.

FIG. 9 is a schematic illustration of the redox-responsive NPs for systemic delivery siRNA to target liver genes involved in NASH.

FIGS. 10A-10B are spectra and profiles of PDSA8-polymer. FIG. 10A is an ¹H-NMR spectrum of PDSA8-1 polymer in DMSO-d6. FIG. 10B is a GPC profile of PDSA8-1 polymer incubated in 9:1 (v/v) DMF/H2O mixture containing 10 mM GSH for 4 h.

FIGS. 11A-11B are a micrograph and a graph respectively, showing morphology and distribution of the Luc siRNA loaded PDSA8-1 NPs in PBS solution. FIG. 11A is an electron micrograph showing the morphology of the Luc siRNA loaded PDSA8-1 NPs in PBS solution (pH 7.4). FIG. 11B is a graph showing the size distribution of the Luc siRNA loaded PDSA8-1 NPs in PBS solution (pH 7.4).

FIGS. 12A-12B are a graph and an electron micrograph showing size change and morphology of Luc siRNA loaded PDSA8-1 NPs. FIG. 12A is a graph showing the size change of the Luc siRNA loaded PDSA8-1 NPs with the presence of 10 mM GSH. FIG. 12B is an electron micrograph showing the morphology of the Luc siRNA loaded PDSA8-1 NPs incubated in PBS solution containing 10 mM GSH for 4 h.

FIG. 13 is a graph showing cumulative siRNA release from the DY-547 siRNA loaded PDSA8-1 NPs incubated in PBS solution containing GSH at different concentrations.

FIGS. 14A-14B are fluorescent images of Luc-HeLa cells incubated with the DY-547 siRNA loaded PDSA8-1 NPs for 1 h (FIG. 14A) and 4 h (FIG. 14B).

FIG. 15 is a graph showing Luc expression in Luc-HeLa cells treated with the Luc siRNA loaded PDSA or PLGA NPs at 10 nM siRNA dose.

FIG. 16 is a blot showing TAZ expression in AML12 cells treated with the TAZ siRNA loaded PDSA8-1 NPs at different siRNA doses.

FIG. 17 is a graph showing the pharmacokinetics of naked DY-647 siRNA and DY647-siRNA loaded PDSA8-1 NPs.

FIGS. 18A-18B are a fluorescent image and a graph showing the biodistribution of the DY677-siRNA loaded PDSA8-1 NPs in main organs of the normal (Chow) and NASH model (FPC) mice sacrificed 24 h post injection of the NPs.

FIG. 19 are images showing the accumulation of the DY677-siRNA loaded PDSA8-1 NPs in liver of the normal mice sacrificed 8, 12, 24, 36, 48, 72 and 96 h post injection of the NPs. The highest amount is shown in the center of the liver tissue section for the 8-hour time period, while the least amount is shown for the tissue section at the 96-hour period.

FIGS. 20A-20B show that plasma IHH increases in NASH mice and that silencing TAZ decreases plasma IHH. FIG. 20A: IHH was measured in the plasma of C57BL/6J mice fed with chow or FPC diet for 28 weeks. n=4, p<0.001. FIG. 20B: C57BL/6J mice were treated with AAV8-H1-shTaz or control vector and then fed the FPC diet for 16 weeks. Mouse IHH was measured in the plasma. n=5, p<0.01.

FIG. 21 shows that plasma IHH increases in NASH patients. Human IHH was measured in control (n=15), NASH (n=14), and steatosis (n=13) human plasma. *p<0.05, **p<0.01.

FIGS. 22A-22B show that serum IHH increases in NASH patients. FIG. 22A: IHH was measured in NAFLD (n=18) and NASH (n=36) human serum. *p<0.05. FIG. 22B: Correlation analysis between serum IHH and ALT. R2=0.31, p<0.001.

### DETAILED DESCRIPTION

The methods of the present disclosure assay the levels of IHH in a sample (e.g., a plasma or serum sample) taken from a patient. The levels of IHH in the sample can be used for diagnosing, or assessing the severity of, a liver condition. Based on the levels of IHH, a liver condition may be diagnosed or predicted, and then the subject may be treated. The IHH level may also be used as an indicator of the efficacy of a therapeutic intervention for treating a liver condition. For patients undergoing a treatment or therapy, based on the IHH levels, the treatment or therapeutic intervention may be continued when it is effective, or altered if ineffective or insufficient.

The level of IHH may refer to the level of the IHH protein or the level of the nucleic acid encoding IHH such as IHH mRNA.

The present methods can diagnose or predict a liver condition in a subject. The present methods may determine/detect the presence, type and/or severity of a liver condition. The method may also identify a subject at risk for developing a liver condition or assess the susceptibility of a subject to a liver condition.

In certain embodiments, the method contains the following steps: (a) obtaining a sample (e.g., a plasma or serum sample, or other samples as discussed herein) from the subject; (b) assaying the level of IHH (protein or nucleic acid such as mRNA) in the sample; and (c) comparing the IHH level in the sample with the level of IHH in a control sample. The subject is diagnosed to have a liver condition (or diagnosed to have an increased risk of a liver condition), if the level of IHH obtained in the sample increases by at least 25% (or at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, about 20% to about 90%, about 50% to about 100%, at least 1 fold, at least 1.5 folds, at least 2 folds, at least 2.5 folds, or at least 3 folds) compared to its level in the control sample.

The present methods may treat a subject with a liver condition or an increased risk of developing a liver condition. When diagnosed with a liver condition, the subject may be treated. Alternatively, when a liver condition is predicted (or when an increased risk of a liver condition is diagnosed), the subject may be treated.

In certain embodiments, the method contains the following steps: (a) obtaining a sample (e.g., a plasma or serum sample, or other samples as discussed herein) from the subject; (b) assaying the level of IHH (protein or nucleic acid such as mRNA) in the sample; (c) comparing the IHH level in the sample with the level of the IHH in a control sample; and (d) treating the subject for a liver condition or an increased risk of a liver condition, if the level of IHH in the sample increases by at least 25% (or at least 5%, at least 10%, at least 15%, at least 20%, at least 30%, at least 35%, at least 40%, at least 45%, at least 50%, at least 55%, at least 60%, at least 65%, at least 70%, at least 75%, at least 80%, at least 85%, at least 90%, about 20% to about 90%, about 50% to about 100%, at least 1 fold, at least 1.5 folds, at least 2 folds, at least 2.5 folds, or at least 3 folds) compared to its level in the control sample.

The level of IHH (protein or nucleic acid such as mRNA) in the sample may increase by at least or about 5%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 100%, about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, about 10% to about 90%, about 12.5% to about 80%, about 20% to about 70%, about 25% to about 60%, about 25% to about 50%, about 5% to about 50 folds, about 10% to about 50 folds, about 15% to about 50 folds, about 20% to about 50 folds, about 25% to about 50 folds, about 30% to about 50 folds, about 5% to about 40 folds, about 10% to about 40 folds, about 15% to about 40 folds, about 20% to about 40 folds, about 25% to about 40 folds, about 30% to about 40 folds, about 5% to about 30 folds, about 10% to about 30 folds, about 15% to about 30 folds, about 20% to about 30 folds, about 25% to about 30 folds, about 30% to about 30 folds, about 5% to about 20 folds, about 10% to about 20 folds, about 15% to about 20 folds, about 20% to about 20 folds, about 25% to about 20 folds, about 30% to about 20 folds, about 5% to about 15 folds, about 10% to about 15 folds, about 15% to about 15 folds, about 20% to about 15 folds, about 25% to about 15 folds, about 30% to about 15 folds, about 5% to about 10 folds, about 10% to about 10 folds, about 15% to about 10 folds, about 20% to about 10 folds, about 25% to about 10 folds, about 30% to about 10 folds, about 5% to about 8 folds, about 10% to about 8 folds, about 15% to about 8 folds, about 20% to about 8 folds, about 25% to about 8 folds, about 30% to about 8 folds, about 5% to about 6 folds, about 10% to about 6 folds, about 15% to about 6 folds, about 20% to about 6 folds, about 25% to about 6 folds, about 30% to about 6 folds, about 5% to about 5 folds, about 10% to about 5 folds, about 15% to about 5 folds, about 20% to about 5 folds, about 25% to about 5 folds, about 30% to about 5 folds, about 5% to about 3 folds, about 10% to about 3 folds, about 15% to about 3 folds, about 20% to about 3 folds, about 25% to about 3 folds, about 30% to about 3 folds, about 5% to about 2 folds, about 10% to about 2 folds, about 15% to about 2 folds, about 20% to about 2 folds, about 25% to about 2 folds, about 30% to about 2 folds, about 5% to about 100%, about 10% to about 100%, about 15% to about 100%, about 20% to about 100%, about 25% to about 100%, about 30% to about 100%, at least or about 2 folds, at least or about 2.5 folds, at least or about 3 folds, at least or about 3.5 folds, at least or about 4 folds, at least or about 5 folds, at least or about 6 folds, at least or about 7 folds, at least or about 8 folds, at least or about 9 folds, at least or about 10 folds, at least or about 1.1 folds, at least or about 1.2 folds, at least or about 1.3 folds, at least or about 1.4 folds, at least or about 1.5 folds, at least or about 1.6 folds, at least or about 1.8 folds, at least or about 11 folds, at least or about 12 folds, at least or about 13 folds, at least or about 14 folds, at least or about 15 folds, at least or about 16 folds, at least or about 17 folds, at least or about 18 folds, at least or about 19 folds, at least or about 20 folds, at least or about 25 folds, at least or about 30 folds, at least or about 35 folds, at least or about 40 folds, at least or about 45 folds, at least or about 50 folds, at least or about 60 folds, at least or about 70 folds, at least or about 80 folds, at least or about 90 folds, at least or about 100 folds, from about 1.1 folds to about 10 folds, from about 1.1 folds to about 5 folds, from about 1.5 folds to about 5 folds, from about 2 folds to about 5 folds, from about 3 folds to about 4 folds, from about 5 folds to about 10 folds, from about 5 folds to about 20 folds, from about 10 folds to about 15 folds, from about 10 folds to about 20 folds, compared to the level(s) in the control sample.

The control sample may be from a healthy subject or a plurality of healthy subjects. The control sample may be from a subject who does not have the liver condition or a plurality of healthy subjects who do not have the liver condition.

The present method measures the level of IHH in a biological sample. Samples can include any biological sample from which the level or amount of IHH can be assayed.

In certain embodiments, the sample is a body fluid. For example, the body fluid may include, but are not limited to, serum, plasma, blood, whole blood and derivatives thereof, urine, tears, saliva, sweat, cerebrospinal fluid (CSF), oral mucus, vaginal mucus, seminal plasma, semen, prostatic fluid, excreta, ascites, lymph, bile, and amniotic fluid. In certain embodiments, the biological sample is plasma or serum.

In certain embodiment, samples may include, but are not limited to, liver tissue, bone marrow, endothelium, skin, hair, hair follicles, epithelial tissues, as well as other samples or biopsies. In certain embodiments, the biological sample is liver tissue.

The sample may be obtained at any time point. The sample may be obtained at any time point before or after the treatment or therapy, such as about 10 minutes, about 30 minutes, about 1 hour, about 2 hours, about 3 hours, about 4 hours, about 5 hours, about 6 hours, about 7 hours, about 8 hours, about 10 hours, about 12 hours, about 15 hours, about 18 hours, about 20 hours, about 22 hours, about 1 day, about 2 days, about 3 days, about 4 days, about 5 days, about 6 days, about 1 week, about 2 weeks, about 3 weeks, about 1 month, about 2 months, about 3 months, about 4 months, about 5 months, about 6 months, about 1 year, about 2 years, about 3 years, about 5 years or longer before or after the start of the treatment or therapy. The time point may also be earlier or later.

The level or amount of IHH in a patient sample can be compared to a reference level or amount of IHH present in a control sample.

The control sample may be from a patient or patients with a liver condition. The control sample may be from a healthy subject or from healthy subjects. In other embodiments, a control sample is taken from a patient prior to treatment with a therapeutic intervention, or a sample taken from an untreated patient. In certain embodiments, a control sample is from a subject without a liver condition. Reference levels for IHH can be determined by determining the level of IHH in a sufficiently large number of samples obtained from normal, healthy control subjects to obtain a pre-determined reference or threshold value. A reference level can also be determined by determining the level of IHH in a sample from a patient prior to treatment. Reference level information and methods for determining reference levels can be obtained from publicly available databases, as well as other sources.

Also encompassed by the present disclosure is a method for assessing the efficacy of a treatment or therapy for a liver condition in a patient. The method may contain the following steps: (a) obtaining a first sample from the patient before initiation of the treatment or therapy (or at a first time point after initiation of the treatment or therapy, or when the treatment or therapy is initiated); (b) assaying the level of IHH (protein or nucleic acid such as mRNA) in the first sample; (c) obtaining a second sample from the patient after initiation of the treatment or therapy (or at a second time point after initiation of the treatment or therapy); (d) assaying the level of the IHH (protein or nucleic acid such as mRNA) in the second sample; (e) comparing the IHH level in the first sample with the IHH level in the second sample. If the level of IHH obtained in the second sample decreases by at least or about 5%, at least or about 10%, at least or about 15%, at least or about 20%, at least or about 25%, at least or about 30%, at least or about 35%, at least or about 40%, at least or about 45%, at least or about 50%, at least or about 55%, at least or about 60%, at least or about 65%, at least or about 70%, at least or about 75%, at least or about 80%, at least or about 85%, at least or about 90%, at least or about 95%, at least or about 100%, about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, about 10% to about 90%, about 12.5% to about 80%, about 20% to about 70%, about 25% to about 60%, about 25% to about 50%, about 5% to about 50 folds, about 10% to about 50 folds, about 15% to about 50 folds, about 20% to about 50 folds, about 25% to about 50 folds, about 30% to about 50 folds, about 5% to about 40 folds, about 10% to about 40 folds, about 15% to about 40 folds, about 20% to about 40 folds, about 25% to about 40 folds, about 30% to about 40 folds, about 5% to about 30 folds, about 10% to about 30 folds, about 15% to about 30 folds, about 20% to about 30 folds, about 25% to about 30 folds, about 30% to about 30 folds, about 5% to about 20 folds, about 10% to about 20 folds, about 15% to about 20 folds, about 20% to about 20 folds, about 25% to about 20 folds, about 30% to about 20 folds, about 5% to about 15 folds, about 10% to about 15 folds, about 15% to about 15 folds, about 20% to about 15 folds, about 25% to about 15 folds, about 30% to about 15 folds, about 5% to about 10 folds, about 10% to about 10 folds, about 15% to about 10 folds, about 20% to about 10 folds, about 25% to about 10 folds, about 30% to about 10 folds, about 5% to about 8 folds, about 10% to about 8 folds, about 15% to about 8 folds, about 20% to about 8 folds, about 25% to about 8 folds, about 30% to about 8 folds, about 5% to about 6 folds, about 10% to about 6 folds, about 15% to about 6 folds, about 20% to about 6 folds, about 25% to about 6 folds, about 30% to about 6 folds, about 5% to about 5 folds, about 10% to about 5 folds, about 15% to about 5 folds, about 20% to about 5 folds, about 25% to about 5 folds, about 30% to about 5 folds, about 5% to about 3 folds, about 10% to about 3 folds, about 15% to about 3 folds, about 20% to about 3 folds, about 25% to about 3 folds, about 30% to about 3 folds, about 5% to about 2 folds, about 10% to about 2 folds, about 15% to about 2 folds, about 20% to about 2 folds, about 25% to about 2 folds, about 30% to about 2 folds, about 5% to about 100%, about 10% to about 100%, about 15% to about 100%, about 20% to about 100%, about 25% to about 100%, about 30% to about 100%, at least or about 2 folds, at least or about 2.5 folds, at least or about 3 folds, at least or about 3.5 folds, at least or about 4 folds, at least or about 5 folds, at least or about 6 folds, at least or about 7 folds, at least or about 8 folds, at least or about 9 folds, at least or about 10 folds, at least or about 1.1 folds, at least or about 1.2 folds, at least or about 1.3 folds, at least or about 1.4 folds, at least or about 1.5 folds, at least or about 1.6 folds, at least or about 1.8 folds, at least or about 11 folds, at least or about 12 folds, at least or about 13 folds, at least or about 14 folds, at least or about 15 folds, at least or about 16 folds, at least or about 17 folds, at least or about 18 folds, at least or about 19 folds, at least or about 20 folds, at least or about 25 folds, at least or about 30 folds, at least or about 35 folds, at least or about 40 folds, at least or about 45 folds, at least or about 50 folds, at least or about 60 folds, at least or about 70 folds, at least or about 80 folds, at least or about 90 folds, at least or about 100 folds, from about 1.1 folds to about 10 folds, from about 1.1 folds to about 5 folds, from about 1.5 folds to about 5 folds, from about 2 folds to about 5 folds, from about 3 folds to about 4 folds, from about 5 folds to about 10 folds, from about 5 folds to about 20 folds, from about 10 folds to about 15 folds, from about 10 folds to about 20 folds, compared to the level of IHH obtained in the first sample, the therapy is considered to be effective. An effective treatment or therapy may be continued, or discontinued if the patient's condition has improved and is no longer in need of treatment. An ineffective treatment may be altered or modified, or replaced with other treatment.

The present methods can include the steps of measuring the level of IHH in a sample from a patient receiving a treatment or therapeutic intervention, and comparing the measured level to a reference level or the level of IHH in a control sample. The measured level of the IHH is indicative of the therapeutic efficacy of the therapeutic intervention.

Based on the measured IHH levels, therapy may be continued or altered, e.g., by change of the dose or dosing frequency, or by addition of other active agents, or change of therapeutic regimen altogether.

The present disclosure also encompasses a method of predicting or assessing the severity of a liver condition in a patient. In one embodiment, the method comprises measuring the level of IHH in a biological sample from a patient; and comparing the measured level to a reference level or the level of the IHH in a control sample, where the measured level of the IHH is indicative of the severity of a liver condition in the patient. In other embodiments, an increase (as described herein) in the level of IHH is indicative of the level of severity of a liver condition in the patient.

The level of IHH in a patient may be compared with a reference value, where the reference value is based on the level of IHH in a subject without a liver condition, and/or based on the level of IHH in an unaffected individual or in unaffected individuals, and/or based on the level of IHH in the patient before, after and/or during therapy. The changes in the level of IHH may be used to alter or direct therapy, including, but not limited to, initiating, altering or stopping the therapy.

The present method may also comprise treating a liver condition or disease in a subject in need thereof.

### Liver Conditions

The present methods may diagnose or predict any type of liver conditions, including, but not limited to, a fatty liver disease, such as non-alcoholic fatty liver disease (NAFLD; e.g. NAFL or non-alcoholic steatohepatitis (NASH)) or alcoholic fatty liver disease (AFLD; e.g., alcoholic steatohepatitis (ASH)). The liver condition may be a fatty liver disease (steatohepatitis), alcoholic steatohepatitis (ASH), non-alcoholic fatty liver disease (NAFLD), non-alcoholic fatty liver (NAFL), non-alcoholic steatohepatitis (NASH), liver fibrosis, or cirrhosis.

In some embodiments, the liver condition is nonalcoholic fatty liver disease (NAFLD). NAFLD refers to a wide spectrum of liver disease ranging from simple fatty liver (steatosis), to nonalcoholic steatohepatitis (NASH), to cirrhosis (irreversible, advanced scarring of the liver). All of the stages of NAFLD have in common the accumulation of fat (fatty infiltration) in the liver cells (hepatocytes). Simple fatty liver is the abnormal accumulation of a certain type of fat, triglyceride, in the liver cells with no inflammation or scarring. In NASH, the fat accumulation is associated with varying degrees of inflammation (hepatitis) and scarring (fibrosis) of the liver. The inflammatory cells can destroy the liver cells (hepatocellular necrosis). NASH can ultimately lead to scarring of the liver (fibrosis) and then irreversible, advanced scarring (cirrhosis). Cirrhosis that is caused by NASH is the last and most severe stage in the NAFLD spectrum. (Mendler, Michel, “Fatty Liver: Nonalcoholic Fatty Liver Disease (NAFLD) and Nonalcoholic Steatohepatitis (NASH),” ed. Schoenfield, Leslie J., MedicineNet.com, Aug. 29, 2005).

Alcoholic liver disease, or alcohol-induced liver disease, encompasses three pathologically distinct liver diseases related to or caused by the excessive consumption of alcohol: fatty liver (steatosis), chronic or acute hepatitis, and cirrhosis. Alcoholic hepatitis can range from a mild hepatitis, with abnormal laboratory tests being the only indication of disease, to severe liver dysfunction with complications such as jaundice (yellow skin caused by bilirubin retention), hepatic encephalopathy (neurological dysfunction caused by liver failure), ascites (fluid accumulation in the abdomen), bleeding esophageal varices (varicose veins in the esophagus), abnormal blood clotting and coma. Histologically, alcoholic hepatitis has a characteristic appearance with ballooning degeneration of hepatocytes, inflammation with neutrophils and sometimes Mallory bodies (abnormal aggregations of cellular intermediate filament proteins). Cirrhosis is characterized anatomically by widespread nodules in the liver combined with fibrosis.

The present methods may be used to diagnose, predict and/or treat an alcoholic liver disease, NAFLD, or any stage thereof, including, for example, steatosis, steatohepatitis, hepatitis, hepatic inflammation, NASH, cirrhosis, or complications thereof.

The treatment may result in a reduction in one, two, three or more of the following: liver fat content, incidence or progression of cirrhosis, incidence of hepatocellular carcinoma, signs of inflammation, e.g., abnormal hepatic enzyme levels (e.g., aspartate aminotransferase AST and/or alanine aminotransferase ALT, or LDH), elevated serum ferritin, elevated serum bilirubin, and/or signs of fibrosis, e.g., elevated TGF-beta levels. Such methods may result, for example, in reduction of AST and/or ALT levels.

The present methods may measure the expression levels of IHH, IHH polynucleotides, IHH nucleotides and IHH nucleic acids, as well as variants, homologues, derivatives and fragments of any of these. The terms “IHH polynucleotide”, “IHH nucleotide” and “IHH nucleic acid,” “IHH nucleic acid” may be used interchangeably, and should be understood to specifically include both cDNA and genomic IHH sequences. These terms are also intended to include a nucleic acid sequence capable of encoding an IHH polypeptide and/or a fragment, derivative, homologue or variant.

### Protein-Based Assays

The level of IHH can be detected and/or quantified by any of a number of methods well known to those of skill in the art. The methods may include various immunoassays such as enzyme-linked immunosorbent assay (ELISA), lateral flow immunoassay (LFIA), immunohistochemistry, antibody sandwich capture assay, immunofluorescent assay, Western blot, enzyme-linked immunospot assay (EliSpot assay), precipitation reactions (in a fluid or gel), immunodiffusion, immunoelectrophoresis, radioimmunoassay (RIA), competitive binding protein assays, chemiluminescent assays, and the like. Also included are analytic biochemical methods such as electrophoresis, capillary electrophoresis, high-performance liquid chromatography (HPLC), thin layer chromatography (TLC), hyperdiffusion chromatography, liquid chromatography-tandem mass spectrometry, and the like. IHH may be detected by, for example, mass spectrometry (e.g., LC-MS/MS) and Western blot. U.S. Pat. Nos. 4,366,241; 4,376,110; 4,517,288; and 4,837,168. Methods in Cell Biology Volume 37: Antibodies in Cell Biology, Asai, ed. Academic Press, Inc. New York (1993); Basic and Clinical Immunology 7th Edition, Stites & Terr, eds. (1991).

The level of IHH may be detected by using molecules (e.g., polypeptides, etc.) that bind to IHH. For example, the binding polypeptide may be an antibody or antibody fragment, such as an Fab, F(ab)₂, F(ab′)₂, Fd, or Fv fragment of an antibody. Any of the various types of antibodies can be used for this purpose, including, but not limited to, polyclonal antibodies, monoclonal antibodies, humanized antibodies, human antibodies (e.g., generated using transgenic mice, etc.), single chain antibodies (e.g., single chain Fv (scFv) antibodies), heavy chain antibodies and chimeric antibodies. The antibodies can be from various species, such as rabbits, mice, rats, goats, chickens, guinea pigs, hamsters, horses, sheep, llamas etc.

In certain embodiments, ELISA is used to detect and/or quantify IHH in a sample. The ELISA can be any suitable methods, including, but not limited to, direct ELISA, sandwich ELISA, and competitive ELISA.

In certain embodiments, Western blot (immunoblot) is used to detect and quantify IHH in a sample. The technique may comprise separating sample proteins by gel electrophoresis, transferring the separated proteins to a suitable solid support, and incubating the sample with the antibodies that specifically bind IHH.

The polypeptides that may be used to assay the level of IHH may be derived also from sources other than antibody technology. For example, such binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties. IHH can be used to screen peptide libraries, including phage display libraries, to identify and select peptide binding partners of IHH. Yeast two-hybrid screening methods also may be used to identify polypeptides that bind to IHH.

### Nucleic Acid-Based Assays

The present methods may also assay the presence of, or quantity of, the gene encoding IHH or the gene product. Gene products include nucleic acids (e.g., mRNAs) derived from the gene.

The level of the DNA or RNA (e.g., mRNA) molecules may be determined/detected using routine methods known to those of ordinary skill in the art. The measurement result may be an absolute value or may be relative (e.g., relative to a reference oligonucleotide, relative to a reference mRNA, etc.). The level of the nucleic acid molecule may be determined/detected by nucleic acid hybridization using a nucleic acid probe, or by nucleic acid amplification using one or more nucleic acid primers.

Nucleic acid hybridization can be performed using Southern blots, Northern blots, nucleic acid microarrays, etc.

For example, the DNA encoding IHH in a sample may be evaluated by a Southern blot. Similarly, a Northern blot may be used to detect an IHH mRNA. In one embodiment, mRNA is isolated from a given sample, and then electrophoresed to separate the mRNA species. The mRNA is transferred from the gel to a solid support. Labeled probes are used to identify or quantity IHH nucleic acids.

In certain embodiments, labeled nucleic acids are used to detect hybridization.

Complementary nucleic acids may be labeled by any one of several methods typically used to detect the presence of hybridized polynucleotides. One method of detection is the use of autoradiography. Other labels include ligands that bind to labeled antibodies, fluorophores, chemiluminescent agents, enzymes, and antibodies which can serve as specific binding pair members for a labeled ligand.

The sensitivity of the assays may be enhanced through use of a nucleic acid amplification system that multiplies the target nucleic acid being detected.

Nucleic acid amplification assays include, but are not limited to, the polymerase chain reaction (PCR), reverse transcription polymerase chain reaction (RT-PCR), real-time RT-PCR, quantitative RT-PCR, etc.

Measuring or detecting the amount or level of mRNA in a sample can be performed in any manner known to one skilled in the art and such techniques for measuring or detecting the level of an mRNA are well known and can be readily employed. A variety of methods for detecting mRNAs have been described and may include, Northern blotting, microarrays, real-time PCR, RT-PCR, targeted RT-PCR, in situ hybridization, deep-sequencing, single-molecule direct RNA sequencing (RNAseq), bioluminescent methods, bioluminescent protein reassembly, BRET (bioluminescence resonance energy transfer)-based methods, fluorescence correlation spectroscopy and surface-enhanced Raman spectroscopy (Cissell, K. A. and Deo, S. K. (2009) Anal. Bioanal. Chem., 394:1109-1116).

The present methods may include the step of reverse transcribing RNA when assaying the level or amount of an mRNA of IHH.

These assays of determining/detecting the presence and/or level of IHH may include use of a label(s). The labels can be any material having a detectable physical or chemical property. Thus, a label is any composition detectable by spectroscopic, photochemical, biochemical, immunochemical, electrical, optical or chemical means. Such labels may include, but are not limited to, a fluorescent label, a radiolabel, a chemiluminescent label, an enzyme, a metallic label, a bioluminescent label, a chromophore, biotin etc. For example, a fluorescently labeled or radiolabeled antibody that selectively binds to a polypeptide of the invention may be contacted with a tissue or cell to visualize the polypeptide. In some aspects of the invention, a label may be a combination of the foregoing molecule types.

The level, amount, abundance or concentration of IHH may be measured. The measurement result may be an absolute value or may be relative (e.g., relative to a reference protein or polypeptide, etc.) In certain embodiments, a reduction or decrease in the measured level of IHH relative to the level of IHH in the control sample (e.g., a sample in the patient prior to treatment or an untreated patient) or pre-determined reference value can be indicative of the therapeutic efficacy of the therapeutic intervention. For instance, in such embodiments, when the level of IHH is decreased (or increased) when compared to the level in a control sample or pre-determined reference value in response to a therapeutic intervention, the decrease (or increase) is indicative of therapeutic efficacy of the therapeutic intervention.

The present invention also provides methods for modifying a treatment regimen comprising detecting the level of IHH in a biological sample from a patient receiving the therapeutic intervention and modifying the treatment regimen based on an increase or decrease in the level of the IHH in the biological sample. The methods for modifying the treatment regimen of a therapeutic intervention may comprise the steps of: (a) detecting the level of IHH in a biological sample from a patient receiving the therapeutic intervention; and (b) modifying the treatment regimen based on an increase or decrease in the level of the IHH in the biological sample.

Modifying the treatment regimen can include, but is not limited to, changing and/or modifying the type of therapeutic intervention, the dosage at which the therapeutic intervention is administered, the frequency of administration of the therapeutic intervention, the route of administration of the therapeutic intervention, as well as any other parameters that would be well known by a physician to change and/or modify. For example, where IHH decrease during therapy or match reference levels, the therapeutic intervention is continued. In embodiments where IHH do not decrease during therapy or match reference levels, the therapeutic intervention is modified. In another embodiment, the information regarding the increase or decrease in the level of IHH can be used to determine the treatment efficacy, as well as to tailor the treatment regimens of therapeutic interventions.

### Treatment/Therapeutic Intervention

Based on the levels of IHH, a liver condition may be diagnosed or predicted (a risk of a liver condition assessed), and then the subject may be treated.

The subject may be treated with an inhibitor of TAZ and/or an inhibitor of YAP. For example, a nucleic acid molecule complementary to at least a portion of a human TAZ and/or YAP encoding nucleic acid can be used to inhibit TAZ and/or YAP gene expression. Means for inhibiting gene expression using short RNA molecules, for example, are known. Among these are short interfering RNA (siRNA), small temporal RNAs (stRNAs), and micro-RNAs (miRNAs). Inhibitors of TAZ may function to downregulate TAZ by RNA interference. The anti-TAZ agent may comprise a small interfering RNA (siRNA) or short hairpin RNA (shRNA). A specific example of an anti-TAZ agent includes nucleic acids comprising one or more of the following nucleic acid sequences: SEQ ID NO:1, SEQ ID NO:2, and any of SEQ ID NO:55-SEQ ID NO:72 and SEQ ID NO:81.

An approach for therapy of such disorders is to express anti-sense constructs directed against TAZ and/or YAP. Alternatively, double-stranded (ds) RNA may be used to interfere with gene expression.

Other methods of modulating TAZ gene expression include dominant negative approaches. An example of this approach is utilizing a TAZ mutant, such as TAZ S51A, to block TAZ/TEAD interaction, or a small molecule or mimetic which can block TAZ/TEAD interaction (Zhang H, et al., J Biol Chem. 2009 May 15; 284(20):13355-62). TAZ WW domain mutations also block its binding to some transcriptional factors. Other TAZ peptide inhibitors are described in WO2015063747A2. Yet another approach is to use non-functional variants of TAZ polypeptide that compete with the endogenous gene product resulting in inhibition of function. Inhibitors of TAZ co-factors TEAD1, TEAD2, TEAD3 and TEAD4 can be used.

Any suitable viral knockdown system could be utilized for decreasing TAZ mRNA levels, including AAV, lentiviral vectors, or other suitable vectors, that may be capable of being targeted specifically to the liver.

TAZ gene expression may also be modulated by introducing peptides or small molecules which inhibit gene expression or functional activity. Thus, compounds identified by the assays described herein as binding to or modulating, such as down-regulating, the amount, activity or expression of TAZ polypeptide may be administered to liver hepatocyte cells to prevent the function of TAZ polypeptide.

Alternatively, gene therapy may be employed to control the endogenous production of TAZ by the relevant cells such as liver cells in the subject. For example, a polynucleotide encoding a TAZ siRNA or a portion of this may be engineered for expression in a replication defective retroviral vector.

In some embodiments, the level of TAZ is decreased in a liver cell. Furthermore, in such embodiments, treatment may be targeted to, or specific to, liver cells. The expression of TAZ may be specifically decreased only in diseased liver cells (i.e., those cells which are predisposed to the liver condition, or exhibiting liver disease already), and not substantially in other non-diseased liver cells. In these methods, expression of TAZ may not be substantially reduced in other cells, i.e., cells which are not liver cells. Thus, in such embodiments, the level of TAZ remains substantially the same or similar in non-liver cells in the course of or following treatment.

Liver cell specific reduction of TAZ levels may be achieved by targeted administration, i.e., applying the treatment only to the liver cells and not to other cells. In certain embodiments, down-regulation of TAZ expression in liver cells (and not substantially in other cell or tissue types) is employed. Such methods may use of liver specific expression vectors, for liver specific expression of, for example, siRNAs.

SEQ ID NO:1, SEQ ID NO:2, and any of SEQ ID NO:55-SEQ ID NO:72 and SEQ ID NO:81 may be useful in certain embodiments as primers amplifying Taz or as sequences utilized for designing nucleic acid inhibitors (shRNA or RNAi) of TAZ, as shown in the table below. The primer sequences described herein are shown as DNA sequences; however in certain instances it would be useful to utilize the RNA equivalent, in which the sequence is identical, except the T is replaced with U.

TABLE 1
Taz-specific polynucleotides siRNA
(T can be replaced with U for any of the
primers listed below, in certain instances.)

| | | Primers can |
|---|---|---|
| | | also be |
| | | useful as |
| | | RNA if T is |
| | | replaced |
| | | with U |
| | | (T/U) |
| SEQ ID | TCATTGCGAGATTCGGCTG | T/U |
| NO: 55 | | |
| SEQ ID | GATGAATCCGTCCTCGGTG | T/U |
| NO: 56 | | |
| SEQ ID | GAGGCAAGTTGAAAGGTCAGAGGCA | T/U |
| NO: 57 | | |
| SEQ ID | GCTGCACCACGTTCTGCCTTTGTAC | T/U |
| NO: 58 | | |
| SEQ ID | GGCAATGACGTCCTTAGCTGTTTAG | T/U |
| NO: 59 | | |
| SEQ ID | AGGCAGCTTGGTCCAGGAAGTGATT | T/U |
| NO: 60 | | |
| SEQ ID | ACCTCTTCAACTCTGTCATGAA | T/U |
| NO: 61 | | |
| SEQ ID | CGCCCTTTCTAACCTGGCTGTA | T/U |
| NO: 62 | | |
| SEQ ID | TGCCACCGTTCATCATTTTCCTGCT | T/U |
| NO: 63 | | |
| SEQ ID | TCCCCGAGTCCCCAGAAAGATGAAT | T/U |
| NO: 64 | | |
| SEQ ID | CCAGCTCATGGCGGAAAAAGATCCT | T/U |
| NO: 65 | | |
| SEQ ID | ACCCCAGGAAGGTGATGAATCAGCC | T/U |
| NO: 66 | | |
| SEQ ID | GGGCCTTGCGGACCAAGTGATGAGG | T/U |
| NO: 67 | | |
| SEQ ID | GCCCTTGACTGTTTACTAATAGATA | T/U |
| NO: 68 | | |
| SEQ ID | CCAAATCCATCAGATGAAACCATTT | T/U |
| NO: 69 | | |
| SEQ ID | GCCTGCATTTCTGTGGCAGATA | T/U |
| NO: 70 | | |
| SEQ ID | GCCATGAGCACAGATATGAGATCT | T/U |
| NO: 71 | | |

In certain instances, the following RNA sequences are useful in the present methods: the target sequence of mouse IHH siRNA: UGC GGA CAA UCA UAC AGA ACC AGC A (SEQ ID NO:82); target sequence of mouse IHH siRNA: ACC ACC UUC AGU GAU GUG CUU A (SEQ ID NO:83); a target sequence of human Taz siRNA: GGA UAC UAG UUG UGA AAU GGA AAG A (SEQ ID NO: 84).

Examples of inhibitors of TAZ include antibodies, small molecules, nucleotides and their analogues, including purines and purine analogues, oligonucleotides or proteins which are closely related to a binding partner of TAZ, e.g., a fragment of the binding partner, or small molecules which bind to the TAZ polypeptide but do not elicit a response, so that the activity of the polypeptide is prevented, etc.

Additionally, TAZ polypeptide fragments could be utilized as inhibitors, for example See, WO2015063747A2, which describes peptides that block TAZ/TEAD interaction.

Additionally or alternatively, the subject may be treated with one or more inhibitors including an inhibitor of Indian hedgehog (IHH), an inhibitor of YAP, an inhibitor of TEAD1, an inhibitor of TEAD2, an inhibitor of TEAD3, an inhibitor of TEAD4, or any combination of these.

Additionally or alternatively, the subject may be treated with at least one therapeutic agent for treatment of any of the following conditions including: steatosis hepatis, steatohepatitis, non-alcoholic fatty liver disease, non-alcoholic steatohepatitis, adiposity and combinations thereof. Such antidiabetic drugs and insulin sensitizers include, but are not limited to: rosiglitazone; pioglitazone; losartan; simtuzumab (anti-LOXL2); GR-MD-02; Obeticholic acid (OCA) and combinations thereof.

Additionally or alternatively, the subject may be treated with one or more of the following agents: antioxidants (e.g., Vitamins E and C), insulin sensitizers (Metformin, Pioglitazone, Rosiglitazone, and Betaine), hepatoprotectants, and lipid-lowering agents.

Additionally or alternatively, the subject may be treated with one or more of weight loss agents. Weight loss agents include serotonin and noradrenergic re-uptake inhibitors; noradrenergic re-uptake inhibitors; selective serotonin re-uptake inhibitors; and intestinal lipase inhibitors. Particular weight loss agents include liraglutide, orlistat, sibutramine, methamphetamine, ionamin, phentermine, bupropion, diethylpropion, phendimetrazine, benzphetermine, bromocriptine, lorcaserin, topiramate, or agents acting to modulate food intake by blocking ghrelin action, inhibiting diacylglycerol acyltransferase 1 (DGAT1) activity, inhibiting stearoyl Co A desaturase 1 (SCD1) activity, inhibiting neuropeptide Y receptor 1 function, activating neuropeptide Y receptor 2 or 4 function, or inhibiting activity of sodium-glucose cotransporters 1 or 2.

Additionally or alternatively, the subject may be treated with one or more of the following agents: (1) insulin and insulin analogues; (2) insulin secretagogues, including sulphonylureas (e.g. glipizide) and prandial glucose regulators (sometimes called “short-acting secretagogues”), such as meglitinides (e.g. repaglinide and nateglinide); (3) agents that improve incretin action, for example dipeptidyl peptidase IV (DPP-4) inhibitors (e.g. vildagliptin, saxagliptin, and sitagliptin), and glucagon-like peptide-1 (GLP-1) agonists (e.g. exenatide); (4) insulin sensitizing agents including peroxisome proliferator activated receptor gamma (PPARγ) agonists, such as thiazolidinediones (e.g., pioglitazone and rosiglitazone), and agents with any combination of PPAR alpha, gamma and delta activity; (5) agents that modulate hepatic glucose balance, for example biguanides (e.g., metformin), fructose 1,6-bisphosphatase inhibitors, glycogen phosphorylase inhibitors, glycogen synthase kinase inhibitors, and glucokinase activators; (6) agents designed to reduce/slow the absorption of glucose from the intestine, such as alpha-glucosidase inhibitors (e.g., miglitol and acarbose); and (7) agents which antagonize the actions of, or reduce secretion of, glucagon, such as amylin analogues (e.g., pramlintide); (7) agents that prevent the reabsorption of glucose by the kidney, such as sodium-dependent glucose transporter 2 (SGLT-2) inhibitors (e.g., dapagliflozin); (8) agents designed to treat the complications of prolonged hyperglycemia, such as aldose reductase inhibitors (e.g., epalrestat and ranirestat); and agents used to treat complications related to micro-angiopathies; (9) anti-dyslipidemia agents, such as HMG-CoA reductase inhibitors (statins, e.g., rosuvastatin) and other cholesterol-lowering agents; PPARα agonists (fibrates, e.g., gemfibrozil and fenofibrate); bile acid sequestrants (e.g., cholestyramine); (10) cholesterol absorption inhibitors (e.g., plant sterols (i.e., phytosterols), synthetic inhibitors); cholesteryl ester transfer protein (CETP) inhibitors; inhibitors of the ileal bile acid transport system (IBAT inhibitors); bile acid binding resins; nicotinic acid (niacin) and analogues thereof; anti-oxidants, such as probucol; and omega-3 fatty acids; (11) antihypertensive agents, including adrenergic receptor antagonists, such as beta blockers (e.g., atenolol), alpha blockers (e.g., doxazosin), and mixed alpha/beta blockers (e.g., labetalol); adrenergic receptor agonists, including alpha-2 agonists (e.g., clonidine); angiotensin converting enzyme (ACE) inhibitors (e.g., lisinopril), calcium channel blockers, such as dihydropyridines (e.g., nifedipine), phenylalkylamines (e.g., verapamil), and benzothiazepines (e.g., diltiazem); angiotensin II receptor antagonists (e.g., candesartan); aldosterone receptor antagonists (e.g., eplerenone); centrally acting adrenergic drugs, such as central alpha agonists (e.g., clonidine); and diuretic agents (e.g., furosemide); (12) hemostasis modulators, including antithrombotics, such as activators of fibrinolysis; thrombin antagonists; factor VIIa inhibitors; anticoagulants, such as vitamin K antagonists (e.g., warfarin), heparin and low molecular weight analogues thereof, factor Xa inhibitors, and direct thrombin inhibitors (e.g., argatroban); antiplatelet agents, such as cyclooxygenase inhibitors (e.g., aspirin), adenosine diphosphate (ADP) receptor inhibitors (e.g., clopidogrel), phosphodiesterase inhibitors (e.g., cilostazol), glycoprotein IIB/IIA inhibitors (e.g. tirofiban), and adenosine reuptake inhibitors (e.g., dipyridamole); (14) anti-obesity agents, such as appetite suppressant (e.g., ephedrine), including noradrenergic agents (e.g., phentermine) and serotonergic agents (e.g., sibutramine), pancreatic lipase inhibitors (e.g., orlistat), microsomal transfer protein (MTP) modulators, diacyl glycerolacyltransferase (DGAT) inhibitors, and cannabinoid (CB1) receptor antagonists (e.g., rimonabant); (15) feeding behavior modifying agents, such as orexin receptor modulators and melanin-concentrating hormone (MCH) modulators; (16) glucagon like peptide-1 (GLP-1) receptor modulators; (17) neuropeptideY (NPY)/NPY receptor modulators; (18) pyruvate dehydrogenase kinase (PDK) modulators; (19) serotonin receptor modulators; (20) leptin/leptin receptor modulators; (21) ghrelin/ghrelin receptor modulators; or (22) monoamine transmission-modulating agents, such as selective serotonin reuptake inhibitors (SSRI) (e.g., fluoxetine), noradrenaline reuptake inhibitors (NARI), noradrenaline-serotonin reuptake inhibitors (SNRI), triple monoamine reuptake blockers (e.g., tesofensine), and monoamine oxidase inhibitors (MAOI) (e.g., toloxatone and amiflamine), or a pharmaceutically acceptable salt, solvate, solvate of such a salt or a prodrug thereof, optionally together with a pharmaceutically acceptable carrier to a mammal, such as man, in need of such therapeutic treatment.

Additionally or alternatively, the subject may be treated with one or more of the following agents: miR-103/107 antagonists, FXR agonists, Galectin-1/3 agonists, ACC inhibitors, CB-1 inhibitors, Ketohexakinase inhibitors, PDE4 inhbitors, PPARy agonists, A3AR agonists, PDE inhibitors, fluoroketolide, mTOT insulin sensitizers, Caspase inhibitors, Leptin analogs, Galectin-1/3 agonists, SCD1 inhibitors, PPARU6 agonists, LOXL2 antibodies, ASK1 inhibitors, 11β-HSD1 inhibitors, PPAR αδγ agonists, THR-β agonists, Aldosterone inhibitors, FGF-19 analogs, SBAT inhibitors, CCR2/CCR5 inhibitors, GLP-1 agonists, and PPARαγ agonists.

Additionally or alternatively, the subject may be treated with one or more of the following agents: Astra ZenecA AZD4076, Enanta EDP-305, Galectin Therapeutics GR-MD-02, gemcabene, Gilead GS-0976, Gilead GS-9674, Merck MK-4074, pioglitazone, Pfizer PF-06835919, Pfizer CP-945598, Astellas ASP9831, Boehringer Ingelheim BI 1467335, Bristol Myers Squibb BMS-986036, avandia, metformin, losartan, Can-Fite CF102, pentoxifylline, solithromycin, Cirius MSDC-0602K, emricasan, Conatus IDN-6556, metreleptin, aramchol, Genfit GFT505, simtuzumab, Gilead GS-4997, Gilead GS-9450, Roche TRO19622, Roche R005093151, Immuron IMM-124E, obeticholic acid, Inventiva IVA337, Madrigal MGL-3196, MN-001, Mitsubishi Tanabe MT-3995, Mochida EPA-E, NGM Biopharma NGM282, Novartis LMB763, Novartis LJN452, Shire SHP626, cenicriviroc, liraglutide, and saroglitazar.

Additionally or alternatively, the subject may be treated with one or more agents treating dyslipidemia such as statins. Statins include mevastatin, lovastatin, pravastatin, simvastatin, velostatin, dihydrocompactin, fluvastatin, atorvastatin, dalvastatin, carvastatin, crilvastatin, bevastatin, cefvastatin, rosuvastatin, pitavastatin, and glenvastatin.

Additionally or alternatively, the subject may be treated with one or more of the following: a weight loss regimen or program, a diet regimen, an exercise regimen, motivational support (e.g., cognitive behavioral therapy), surgery (e.g., bariatric surgery), and transplantation (e.g., liver transplantation).

When two or more treatment methods or agents are given to the subject, they may be administered simultaneously, sequentially or separately.

When treating the subject, the mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular embodiments, the composition or therapeutic can be administered by injection (see above). In further embodiments of the invention, the composition, therapeutic, or pharmaceutical composition thereof, is administered intravenously, subcutaneously, intramuscularly, intraarterially, intra-articularly (e.g., in arthritis joints), intratumorally, or by inhalation, aerosol delivery. Administration by non-invasive routes (e.g., orally; for example, in a pill, capsule or tablet) is also within the scope of the present invention.

The agent to be administered to the subject may be in a local matter or a systemic manner.

As used herein, “inhibit” or “treat” or “treatment” includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms. Thus, the terms denote that a beneficial result has been conferred on a vertebrate subject with a disorder, disease or symptom, or with the potential to develop such a disorder, disease or symptom.

### Kits

Another aspect of the disclosure is a kit containing a reagent for measuring IHH in a biological sample, instructions for measuring IHH, and instructions for evaluating or monitoring a liver condition in a patient based on the level of the IHH. Also encompassed by the disclosure are kits for assessing or predicting the severity or progression of a liver condition in a subject. The kit may comprise a reagent for measuring IHH in a biological sample, and instructions for assessing severity or progression of a liver condition based on the level of the IHH.

Another aspect of the disclosure is a kit containing a reagent or reagents for measuring IHH in a biological sample, instructions for measuring the IHH, and/or instructions for evaluating or monitoring a liver condition in a patient based on the level of the IHH, and/or instructions for assessing a therapy in a patient. In some embodiments, the kit contains reagents for measuring IHH.

In certain embodiments, the kit comprises antibodies specific to IHH.

In certain embodiments, the kit comprises primers and/or probe for reverse transcribing, amplifying, and/or hybridizing to the mRNAs of IHH. Such kits can further comprise one or more normalization controls and/or a TaqMan probe specific for each mRNA.

Any of the compositions described herein may be comprised in a kit. In one embodiment, the kit contains a reagent for measuring IHH in a biological sample, instructions for measuring the IHH, and instructions for evaluating or monitoring a liver condition in a patient based on the level of the IHH. In some embodiments, the kit contains reagents for measuring the level of IHH. The kit may also be customized for determining the efficacy of therapy for a liver condition, and thus provides the reagents for determining IHH.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed (e.g., sterile, pharmaceutically acceptable buffer and/or other diluents). However, various combinations of components may be comprised in a vial. The kits of the present invention also will typically include a means for containing the nucleic acids, and any other reagent containers in close confinement for commercial sale. Such containers may include injection or blow molded plastic containers into which the desired vials are retained.

Kits may include primers, buffers, and probes along with instructions for determining elevated levels of nucleic acid, proteins, or protein fragments of Indian hedgehog (IHH).

The following are examples of the present invention and are not to be construed as limiting.

### Example 1

TAZ Levels are Increased in the Livers of Humans and Mice with NASH

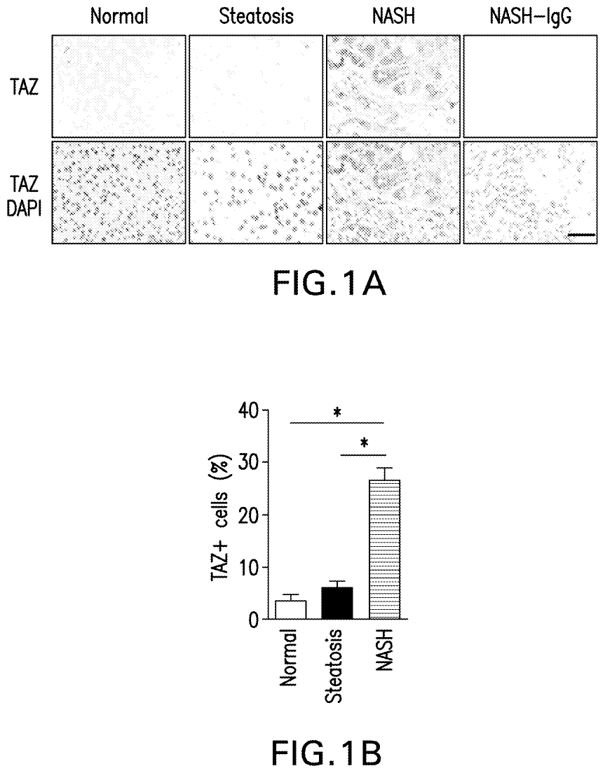

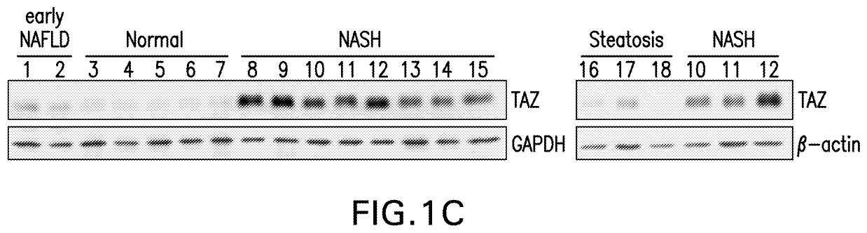

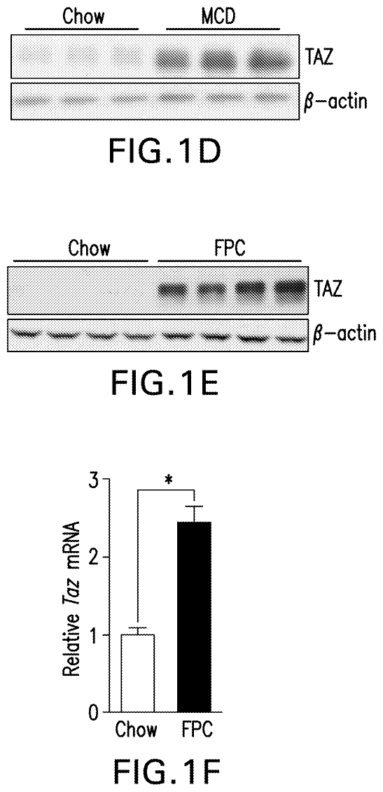

The Hippo pathway transcription factor TAZ contributes to the development of pulmonary fibrosis (Liu et al., 2015). In order to understand whether TAZ plays any role in the development of hepatic fibrosis and NASH progression, NAFLD was evaluated. TAZ immunofluorescence microscopy was conducted on human liver samples from obese individuals with normal, steatotic, and NASH histology. While there was similar TAZ staining in normal and steatotic livers, a significant increase in TAZ staining in the NASH samples was observed (FIGS. 1A-B). The specificity of the anti-human TAZ antibody for immunofluorescence is demonstrated by an siTaz experiment conducted with human HepG2 liver cells. Most of the TAZ-stained cells in human NASH samples were hepatocytes, as identified by HNF4α staining. The Taz protein levels were also analyzed by immunoblot in liver extracts from subjects with NASH vs. early NAFLD and normal liver. The results shown in FIG. 1C illustrate that TAZ was highest in NASH liver.

TAZ expression in various mouse models of NASH was examined. The first mouse model utilized was the methionine/choline-deficient (MCD) diet model, which induces NASH-like liver pathology despite weight loss and insulin sensitivity (Hebbard and George, 2011). TAZ expression was markedly higher in MCD liver compared with control liver (FIG. 1D). Studying TAZ in a NASH model that more closely mimicked the human condition with weight gain and insulin resistance was the next goal. For this purpose, two previously described diet-induced weight-gain models (Charlton et al., 2011; Kohli et al., 2010) were modified to achieve robust NASH features within an experimentally acceptable time frame. For this purpose a new diet was developed that was rich in fructose, palmitate, cholesterol (FPC), and trans-fat, with other features as detailed in Tables 2 and 3. The cholesterol was added in view of links between liver cholesterol and NASH in humans (Ioannou, 2016), and in C57BL/6J mice, the strain used here, a high dietary content of cholesterol is needed to achieve adequately increased cholesterol absorption (Jolley et al., 1999). Additionally, the vitamin E level was lowered in the new diet, compared with that in standard mouse chow diet, because vitamin E has NASH-protective properties (Sanyal et al., 2010).

TABLE 2
Composition of the fructose-palmitate-cholesterol (FPC) diet

| Food Component | g/kg diet |
|---|---|
| Casein, “Vitamin-Free” | 140.0 |
| Sucrose | 341.5 |
| Maltodextrin | 119.6 |
| Vegetable shortening, hydrogenated (Primex) | 190.0 |
| Anhydrous milk fat | 60.0 |
| Palmitic acid (Nu-Chek-Prep N-16A or Sigma | 40.0 |
| W283207) | |
| Cholesterol | 12.5 |
| Cellulose | 50.0 |
| Mineral mix, AIN-76 (170915) | 35.0 |
| Calcium carbonate | 4.0 |
| Vitamin mix, w/o choline, A, D, E (83171) | 5.0 |
| Vitamin E, DL-alpha tocopheryl acetate | 0.1 |
| Vitamin A palmitate | 0.04 |
| Vitamin D3, cholecalciferol | 0.0044 |
| Choline dihydrogen citrate | 2.28 |
| Drinking water | g/L |
| 55% glucose/45% fructose solution (w/w) | 42 |

TABLE 3
Caloric composition of chow, fructose-palmitate-cholesterol (FPC), and
“fast food” (FF) diets. Estimated nutrient data were calculated from
published values and direct analytical testing of raw materials.

| | Chow diet | FPC diet | FF diet |
|---|---|---|---|
| | LabDiet Rodent | Teklad Diets | Test Diet |
| Diet source and # | Diet 20, #5053 | TD.140154 | #1810060 |
| Macronutrients | | | |
| Protein, % by weight | 21.0 | 12.6 | 17.4 |
| Carbohydrate, % by weight | 53.5 | 46.2 | 49.9 |
| Fat, % by weight | 5.0 | 28.8 | 20.0 |
| Protein, % kcal | 24.5 | 10.2 | 15.5 |
| Carbohydrate, % kcal | 62.4 | 37.4 | 44.4 |
| Fat, % kcal | 13.1 | 52.4 | 40.1 |
| Methionine, g/kg | 6.2 | 3.4 | 8.0 |
| Sucrose, % by weight | 3.2 | 34 | 34 |
| Cholesterol, % by weight | 0.01 | 1.25 | 0.2 |
| Vitamin E, IU/kg | 99 | 50 | 50 |
| Choline, mg/kg | 1490 | 915 | 918 |
| SFA, % total fatty acids | 20.7 | 46 | 70 |
| cis-MUFA, % total fatty | 26 | 24 | 26.7 |
| acids | | | |
| cis-PUFA, % total fatty | 53.3 | 4 | 3.3 |
| acids | | | |
| Trans-fats, % total fatty | N/A | 26 | 0 |
| acids | | | |
| Trans-fats, % by weight | N/A | 7 | 0 |
| Palmitic Acid, % by weight | N/A | 8.7 | 5.2 |

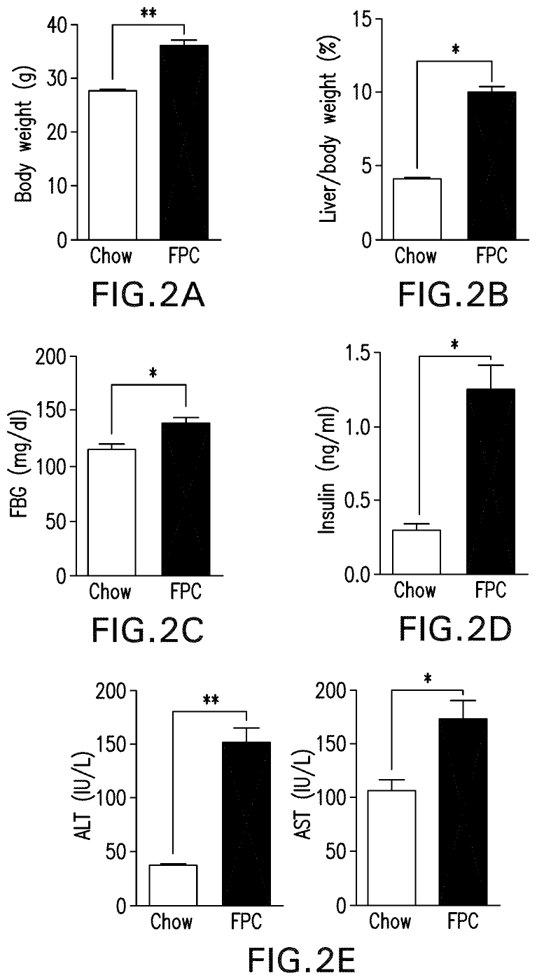

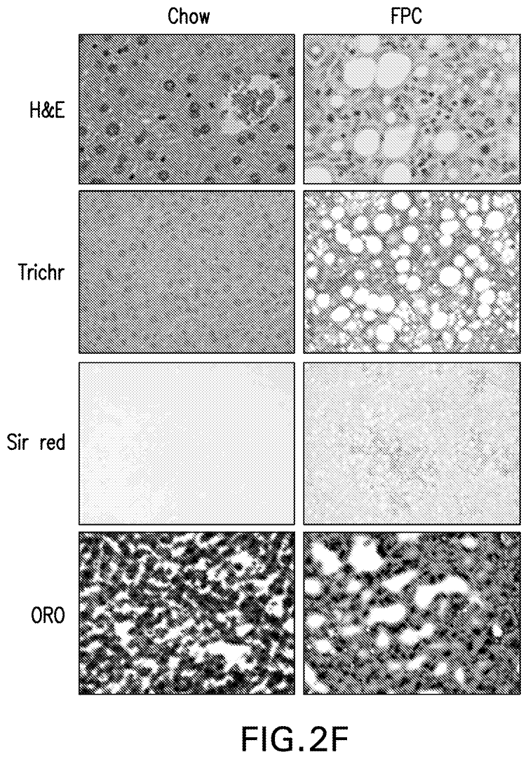

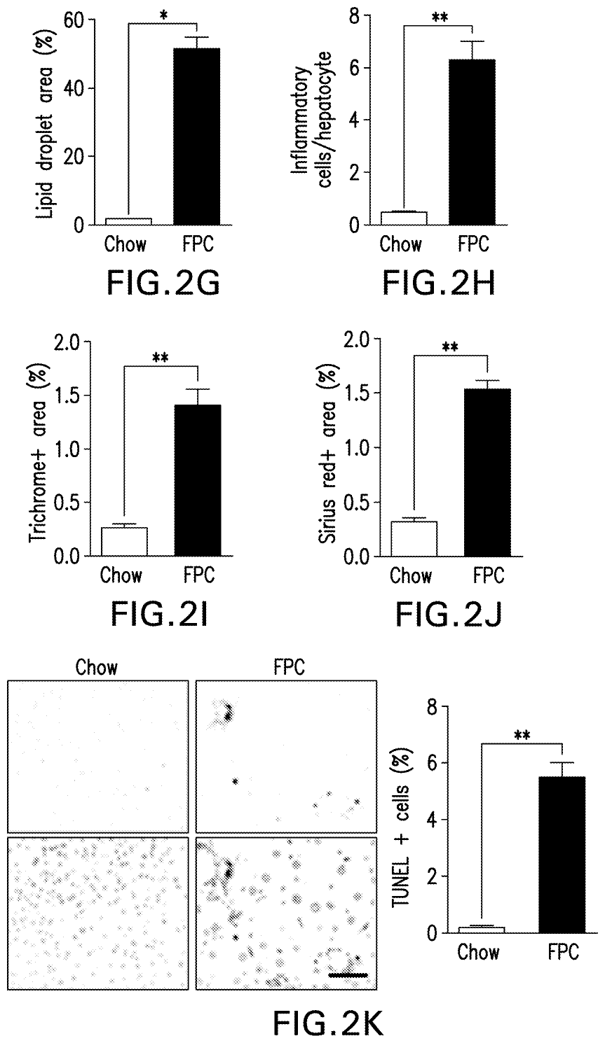

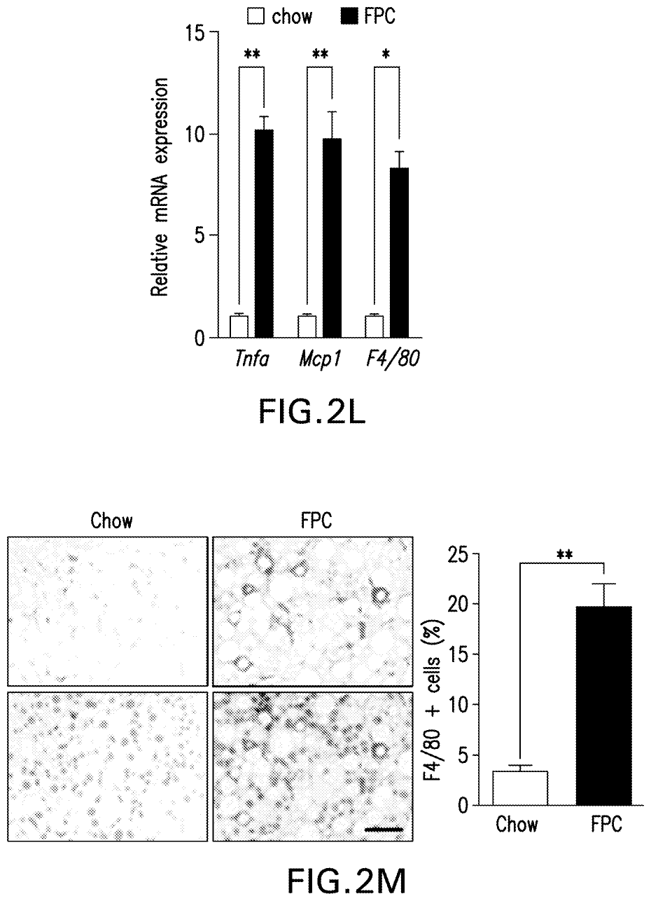

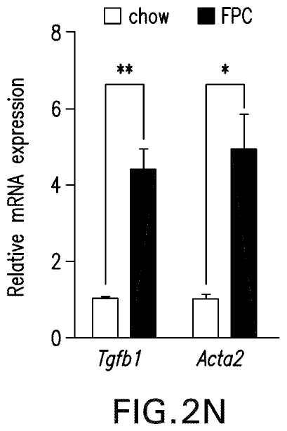

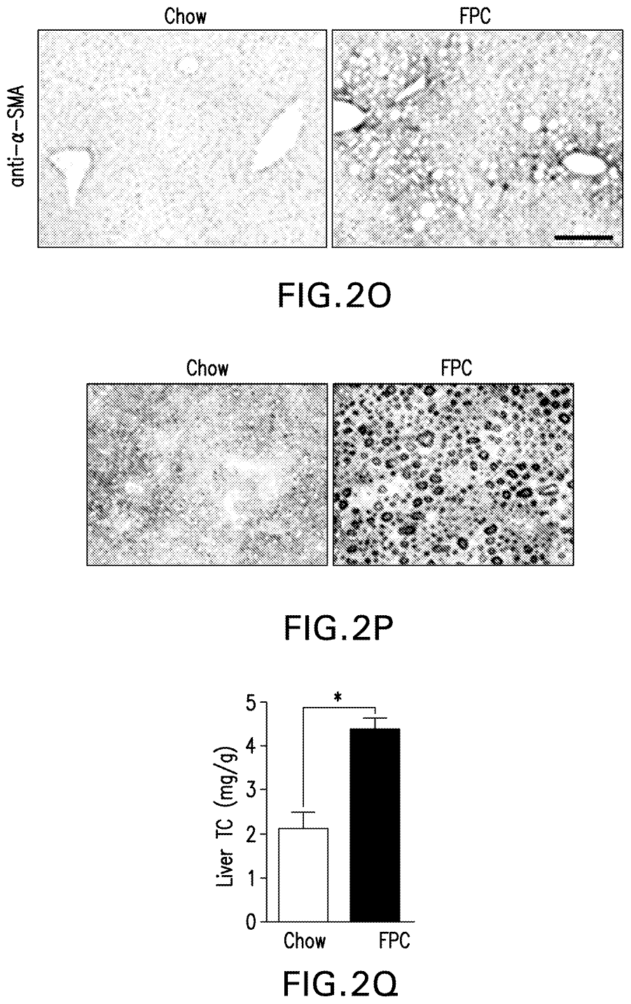

After 16 weeks, FPC-fed mice had higher body weight and liver: body weight ratio than chow-fed mice (FIGS. 2A-2B). Additionally, FPC mice showed significant increases in fasting blood glucose, plasma insulin, and alanine aminotransferase (ALT) and aspartate aminotransferase (AST) levels (FIGS. 2C-2E). In terms of liver histology, lipid droplet area (H&E and Oil Red O [ORO]), inflammatory cells, fibrosis (aniline blue component of trichrome [Trichr] and Sirius red [Sir red]), and cell death (TUNEL) were greater in the livers of FPC-fed mice vs. chow-fed mice (FIGS. 2F-2K). As further evidence of inflammation, FPC liver had elevated liver mRNA levels for Tnfa, Mcp1, F4/80 (Adgre1; macrophages) and a higher percentage of F4/80+ cells (FIGS. 2L-2M). With regard to fibrosis-associated parameters, hepatic Tgfb1 and Acta2 (β-smooth muscle actin, α-SMA) mRNAs were higher in FPC-fed mice compared with chow-fed mice (FIG. 2N), and there was also an increase in α-SMA+ cells (FIG. 2O). Moreover, as designed, liver cholesterol was elevated in the livers of FPC mice (FIGS. 2P-2Q).

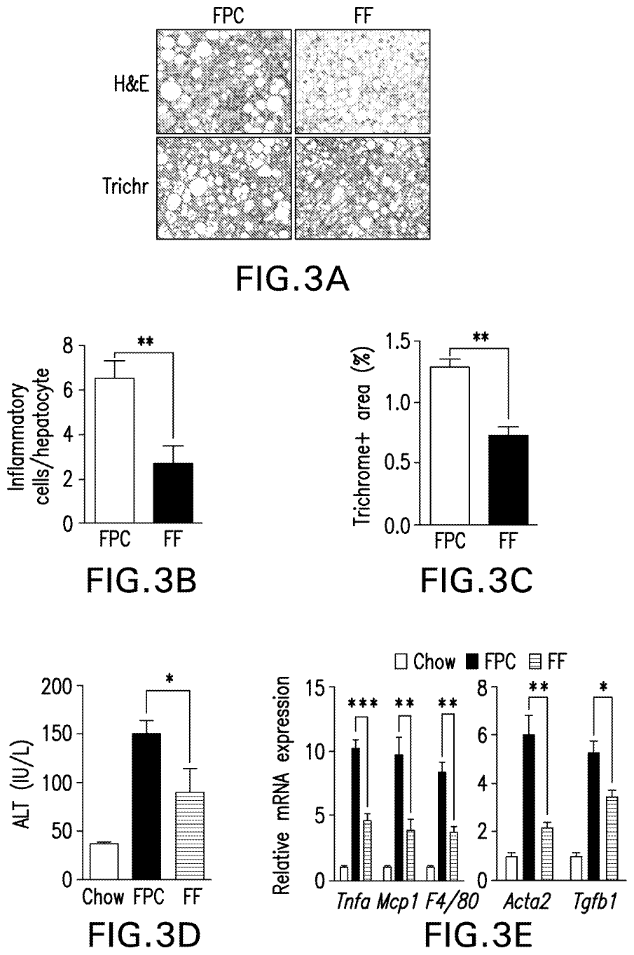

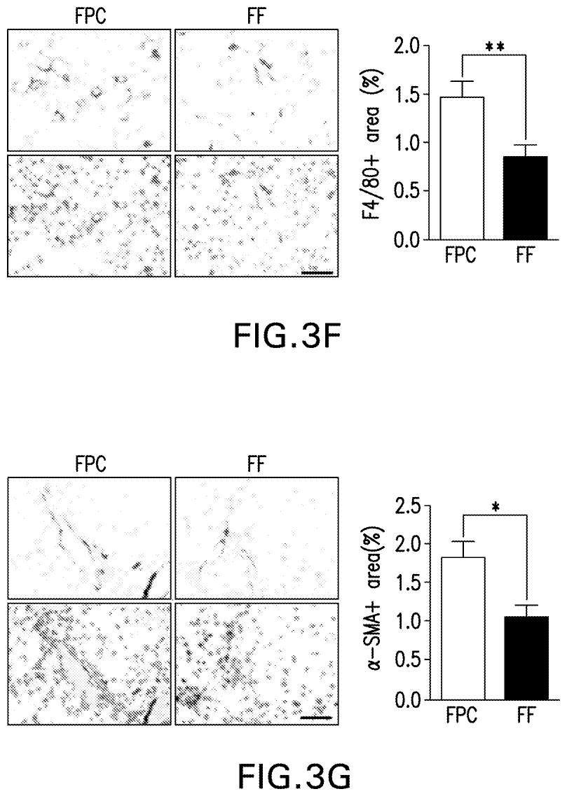

Additional features of the model in terms of blood parameters and liver assays are shown in Table 4 shows hepatic fatty acid changes similar to those reported for human NASH liver (Yamada et al., 2015). Finally, FIGS. 3A-G show a direct comparison of the FPC diet compared with one of the original models referred to as the “fast food” (FF) model (Charlton et al., 2011). Importantly, the livers of FPC mice express high levels of TAZ, including nuclear TAZ (FIG. 1E). Moreover, in view of the fact that the transcriptionally active form of TAZ is non-phosphorylated and nuclear (Liu et al., 2011), these data illustrate that nuclear TAZ was higher in the livers of FPC-fed vs. chow-fed mice and that the ratio of phospho-TAZ:total TAZ was much lower in these livers. Collectively, these data show that TAZ is induced in the livers of humans and mice with NASH and thus raise the possibility that TAZ may be a contributor to the progression from benign steatosis to NASH.

TABLE 4
Saponified fatty acid content of the livers of mice fed the FPC diet for 16 wks (μmol/g
liver). P values were calculated using one-way ANOVA with post-hoc Tukey test.

| | Chow | | | FPC | | | P |
|---|---|---|---|---|---|---|---|
| FA | Average | SD | % of total FA | Average | SD | % of total FA | value |
| C14:1 | 1.581 | 0.3316 | 0.1092 | 3.351 | 0.2631 | 0.0964 | 0.0054 |
| C14 | 12.369 | 2.3932 | 0.8544 | 27.687 | 1.0312 | 0.7962 | 0.0025 |
| C16:1 | 81.675 | 15.447 | 5.6421 | 351.48 | 24.869 | 10.107 | 0.0009 |
| C16 | 233.65 | 29.845 | 16.14 | 420.04 | 9.6215 | 12.079 | 0.0018 |
| C18:3 | 43.305 | 6.1274 | 2.9915 | 10.509 | 0.2135 | 0.3022 | 0.0025 |
| C18:2 | 513.51 | 70.787 | 35.473 | 159.21 | 6.8258 | 4.5782 | 0.0031 |
| C18:1 | 383.23 | 51.698 | 26.473 | 2347.5 | 77.063 | 67.506 | 3E−05 |
| C18 | 49.641 | 11.68 | 3.4292 | 44.349 | 4.7849 | 1.2753 | 0.5944 |
| C20:5 | 13.23 | 2.0541 | 0.9139 | 0.63 | 0.2154 | 0.0181 | 0.0021 |
| C20:4 | 47.481 | 5.6404 | 3.28 | 34.143 | 3.9159 | 0.9818 | 0.0243 |
| C20:1 | 4.317 | 0.8123 | 0.2982 | 63.795 | 6.0916 | 1.8345 | 0.0006 |
| C20 | 0.582 | 0.1372 | 0.0402 | 1.386 | 0.2549 | 0.0399 | 0.0105 |
| C22:6 | 62.589 | 8.0908 | 4.3236 | 10.032 | 1.2551 | 0.2885 | 0.0011 |
| C22:1 | 0 | 0 | 0 | 3.051 | 0.7256 | 0.0877 | 0.0053 |
| C22 | 0.423 | 0.1254 | 0.0292 | 0.36 | 0.0945 | 0.0104 | 0.4897 |
| C24:1 | 0 | | 0 | 0 | | 0 | |
| C24 | 0 | | 0 | 0 | | 0 | |

### TAZ Silencing Reduces Liver Inflammation, Fibrosis, and Cell Death in Mouse Models of NASH

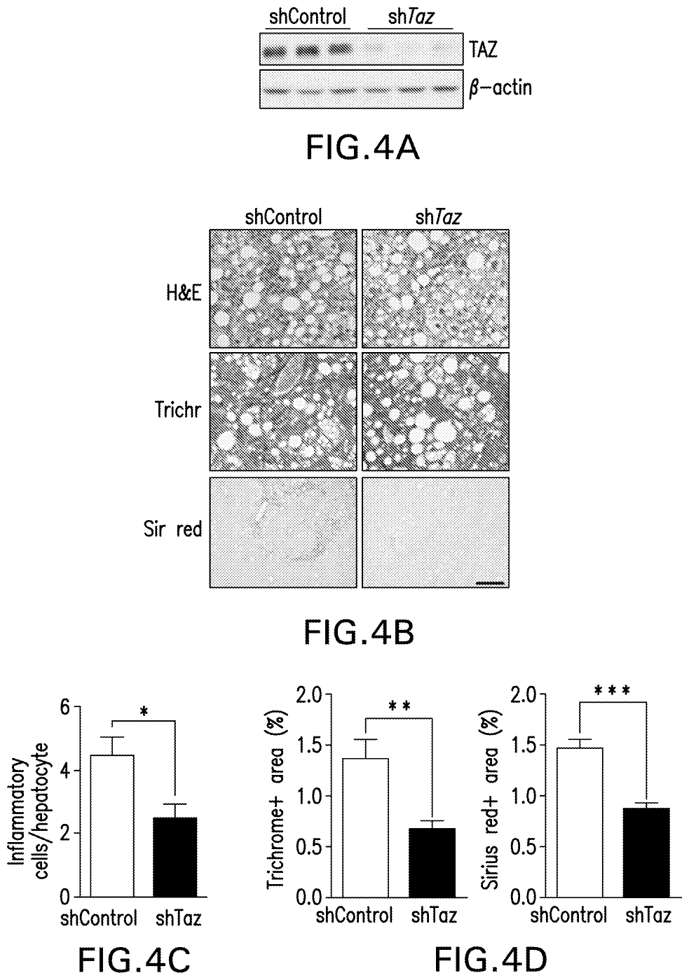

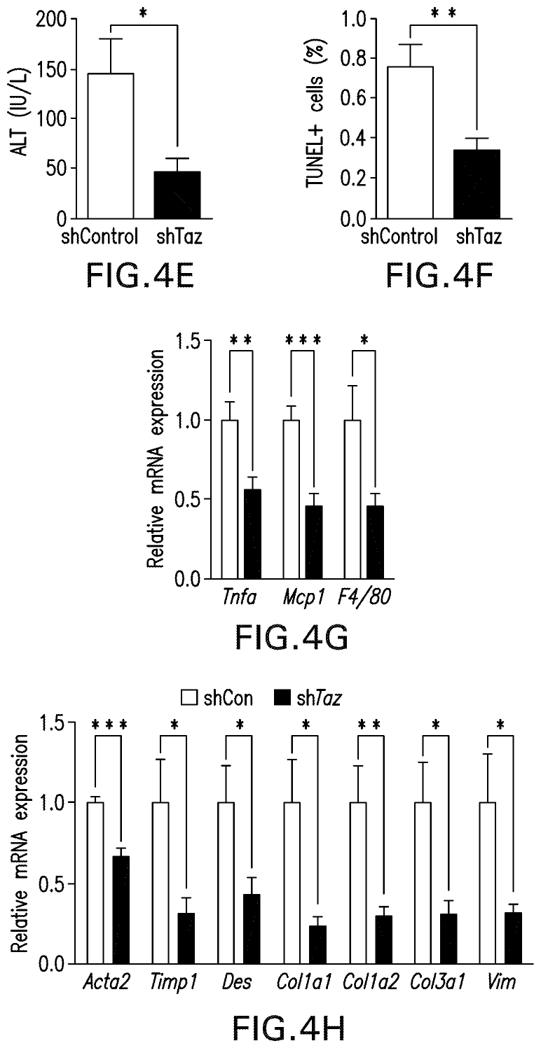

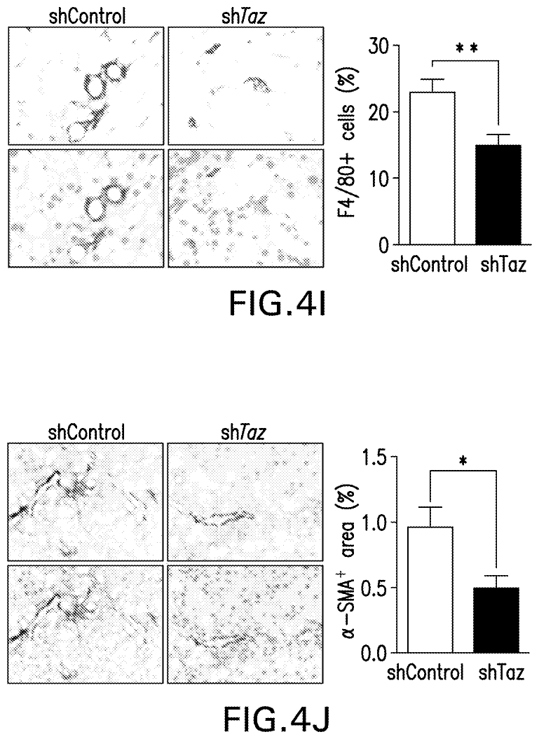

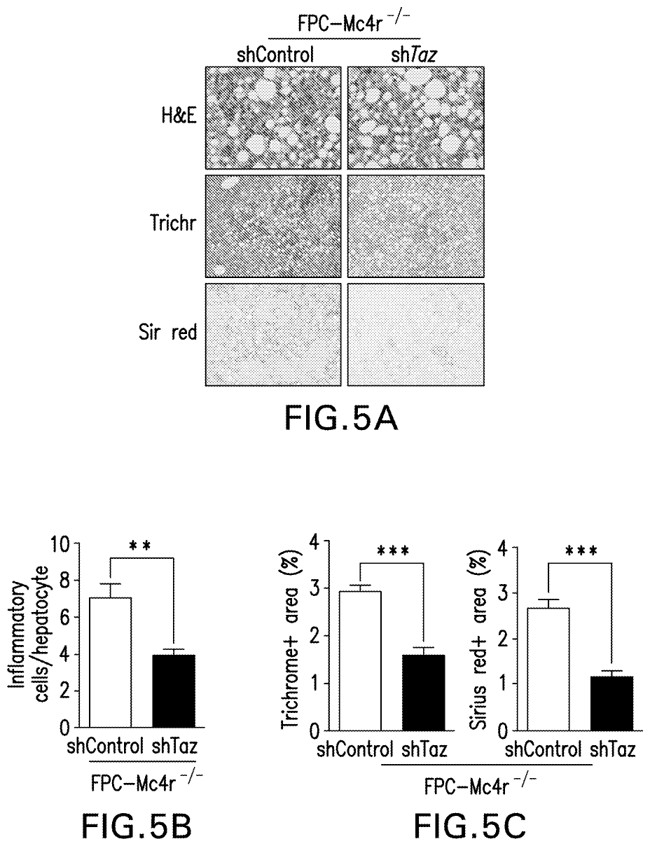

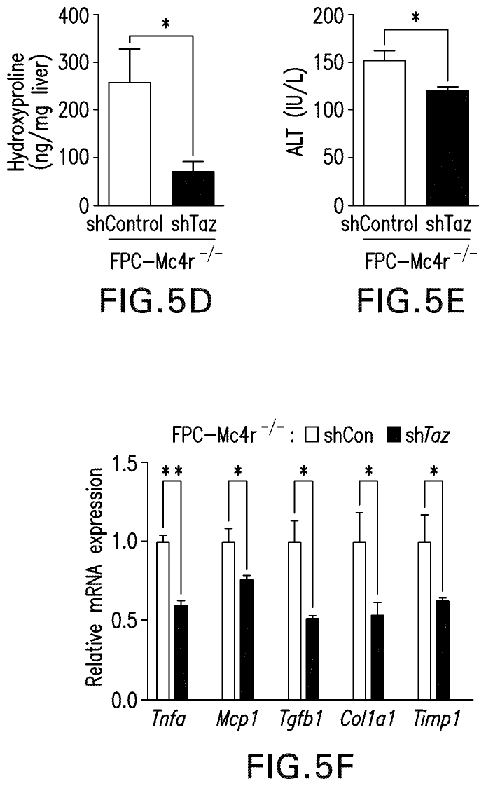

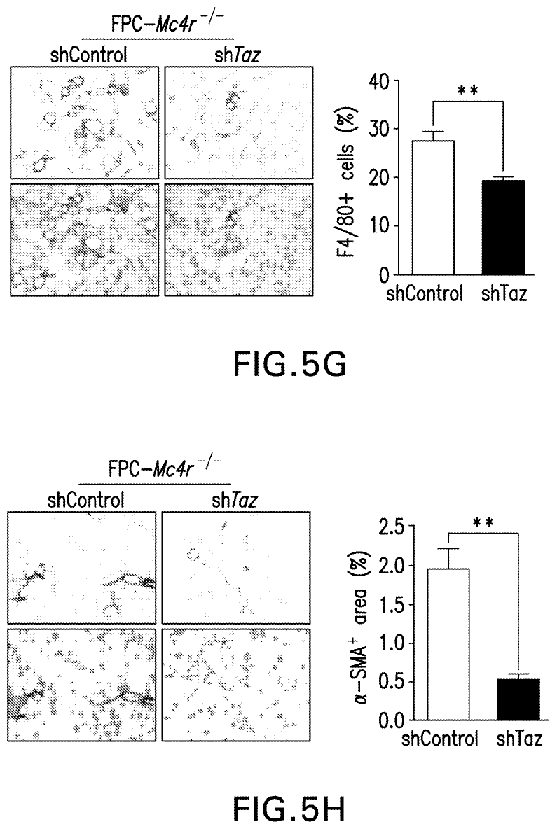

To study TAZ function in NASH development, FPC mice were treated with AAV8-shTaz vs. AAV8-control RNA. AAV8-shTaz led to robust silencing of TAZ (FIG. 4A). Mouse body weight, liver:body weight ratio, fasting blood glucose, plasma insulin, and plasma cholesterol were similar in the Taz shRNA and control groups. Liver sections showed marked reductions in both inflammatory cell infiltration and fibrosis endpoints in the shTaz cohort (FIGS. 4B-D), while steatosis was not affected. Plasma ALT was decreased in shTaz-treated mice (FIG. 4E), and this was associated with a decrease in TUNEL+ and 4-HNE+ liver cells (FIG. 4F), indicating that Taz silencing reduced both cell death and oxidative stress in liver cells. At the mRNA level, Taz silencing caused a robust reduction in the expression of mRNAs related to hepatic inflammation—Tnfa, Mcp1, and F4/80 (Adgre1)—and fibrosis (FIGS. 4G-H), including the NASH-relevant genes Acta2 (α-SMA), Timp1, Des, Col1a1, Col1a2, Co3a1, and Vim (Friedman, 2008; Younossi et al., 2011). These changes were accompanied by decreases in both F4/80+ macrophages and α-SMA+ cells (FIGS. 4I-J). A similar study was also conducted in which hyperphagic Mc4r^{-/-} mice were fed the FPC diet for 16 wks. These mice develop more liver fibrosis compared with FPC-fed WT C57BL/6J mice, and silencing of Taz in the liver of these mice resulted in decreased staining for aniline blue, Sirius red, and α-SMA; lower hydroxyproline content; and decreased liver inflammation (FIGS. 5A-H).

Finally, these results were also confirmed in the MCD model, where shTaz decreased both hepatic inflammation and fibrosis without impacting steatosis and also reduced inflammatory and fibrotic gene expression, α-SMA+ cells, and macrophages in the liver. Thus, in separate models of NASH, hepatic TAZ silencing improved key liver parameters related to inflammation, fibrosis, and cell death without affecting metabolic parameters or steatosis.

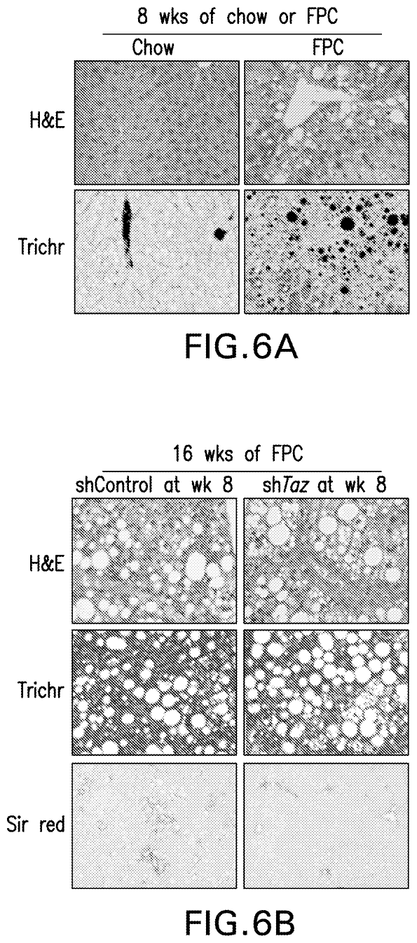

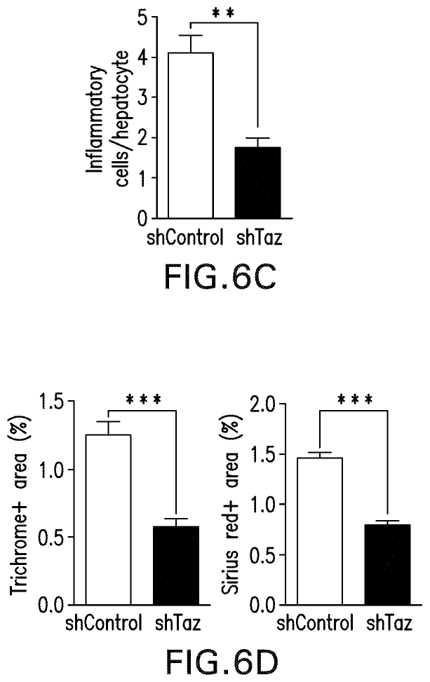

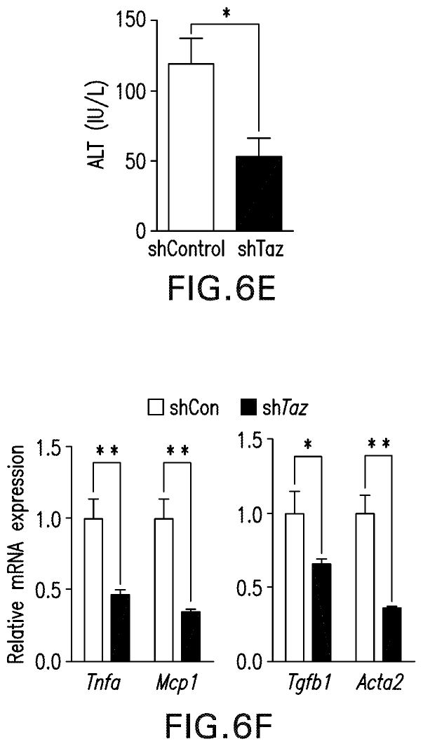

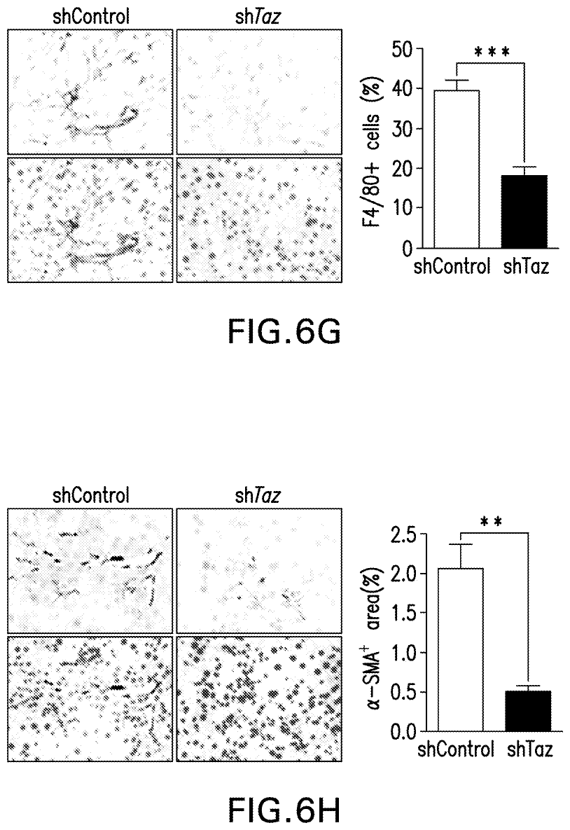

To investigate the role of TAZ specifically in steatosis-to-NASH conversion, mice were first fed the FPC diet for 8 weeks and then injected with AA8-shTaz or control virus, followed by an additional 8 weeks on the diet. Note that 8 weeks of FPC diet caused steatosis but no appreciable inflammation or fibrosis (FIG. 6A). The mice who received shTaz at week 8 showed marked reductions at 16 weeks in inflammatory cells, fibrosis endpoints, plasma ALT, inflammatory- and fibrosis-related genes, F4/80+ macrophages, and α-SMA+ cells, but not steatosis (FIGS. 6B-H). These data suggest that TAZ is particularly important in key processes that promote steatosis-to-NASH progression.

### Hepatocyte TAZ Induces Indian Hedgehog, Which Promotes the Expression of Pro-Fibrotic Genes in Hepatic Stellate Cells

Mechanisms linking TAZ to fibrosis progression were examined. HSCs, the main source of collagen-producing myofibroblasts in NASH-related fibrosis (Mederacke et al., 2013), can be activated by the hedgehog pathway (Syn et al., 2011). In this context, ChIP array data indicated that the gene encoding Indian hedgehog, Ihh, is a TAZ/TEAD target. The new hypothesis that increased TAZ in hepatocytes during NAFLD progression leads to the secretion of Ihh, which then acts on HSCs to promote the expression of pro-fibrotic genes was explored.

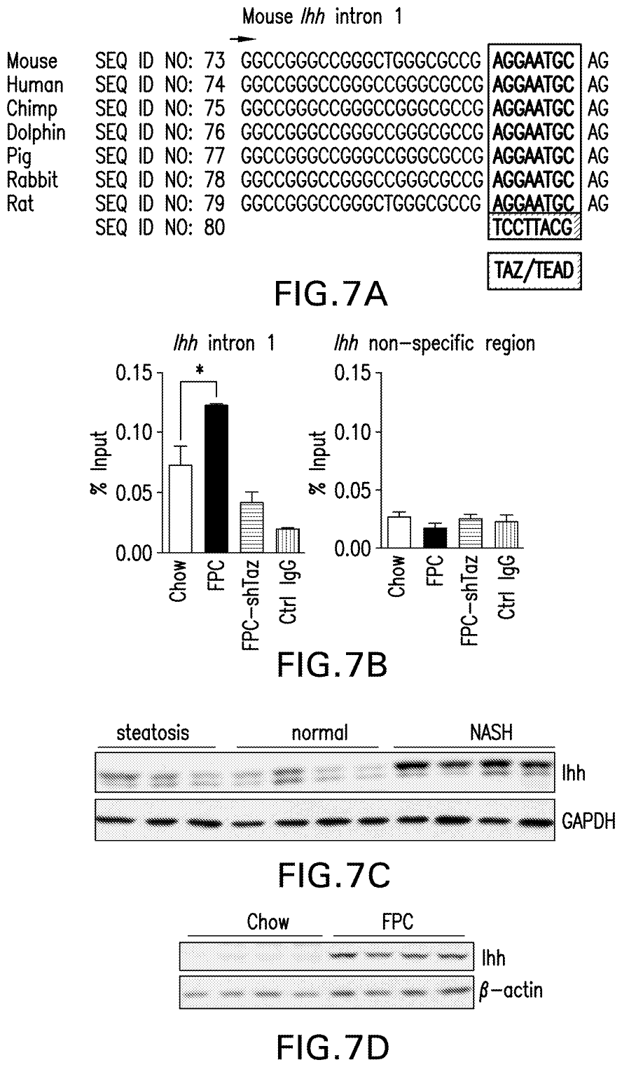

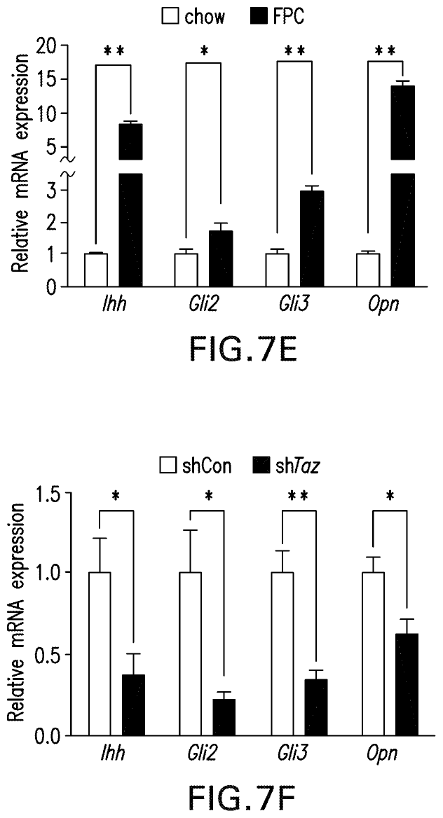

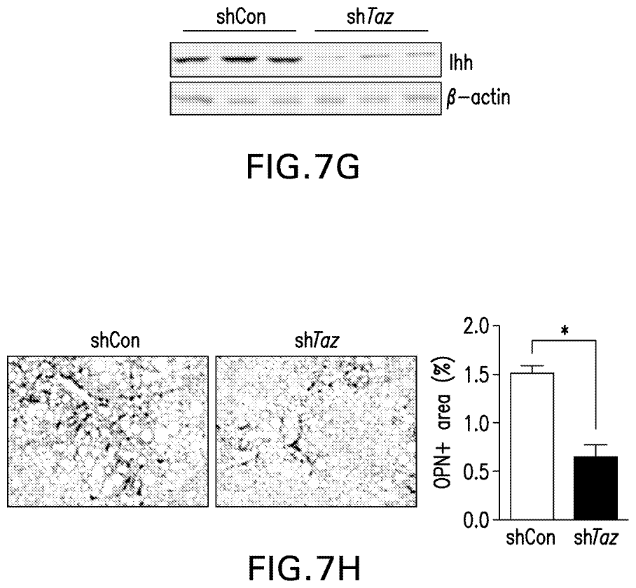

To begin, TAZ ChIP analysis of livers of chow-fed and FPC-fed mice with or without TAZ silencing was conducted, focusing on a TAZ/TEAD consensus sequence in intron 1 of murine Ihh that is conserved among species, including humans (Zanconato et al., 2015) (FIGS. 7A-B). The results show a significant increase in the ChIP signal in the livers of FPC-fed vs. chow-fed mice, which was dependent on anti-TAZ and was not seen when a non-consensus sequence was amplified. Most importantly, the ChIP signal in the livers of FPC mice was lowered to the chow level by TAZ silencing. Thus, TAZ interacts with a TAZ/TEAD consensus sequence in intron 1 of Ihh in the livers of FPC-fed mice.

Next, whether human NASH liver expressed higher levels of Ihh compared with normal and steatotic liver was analyzed. As was the case with TAZ (above), the expression of Ihh was greater in the livers of subjects with NASH compared with normal and steatotic liver (FIG. 7C). Similarly, the livers of FPC-fed mice had markedly higher levels of Ihh compared with the livers of chow-fed mice (FIG. 7D). Next, the chow and FPC liver extracts were compared for gene expression of Ihh and the Ihh pathway downstream genes, Gli2 and Gli3. All three mRNAs were elevated in FPC liver, as was an Ihh target gene, osteopontin (Opn) (Razzaque et al., 2005), which is involved in HSC-induced fibrosis in NASH (Syn et al., 2011) (FIG. 7E). To explore causation with regard to TAZ, these assays were repeated in FPC-fed mice with or without TAZ silencing. All four mRNAs and Ihh protein were substantially lower in the TAZ-silenced mice (FIGS. 7F-G), as was OPN as assessed by immunohistochemistry (FIG. 7H). Thus, TAZ induces transcriptionally active Ihh during NASH progression in FPC-fed mice, and one of the targets of Ihh, Opn, has been linked to NASH fibrosis.

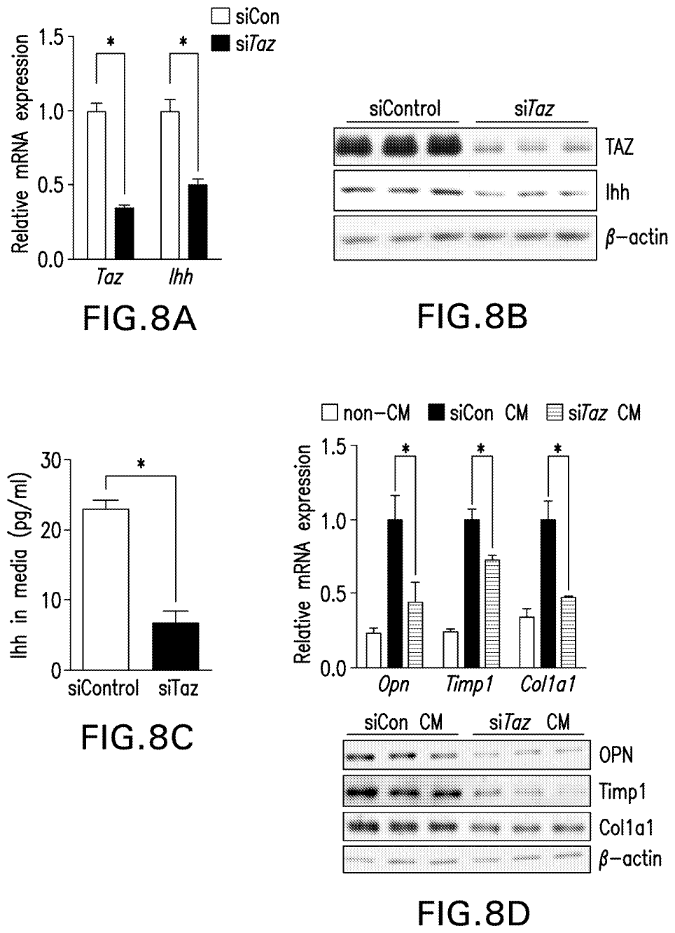

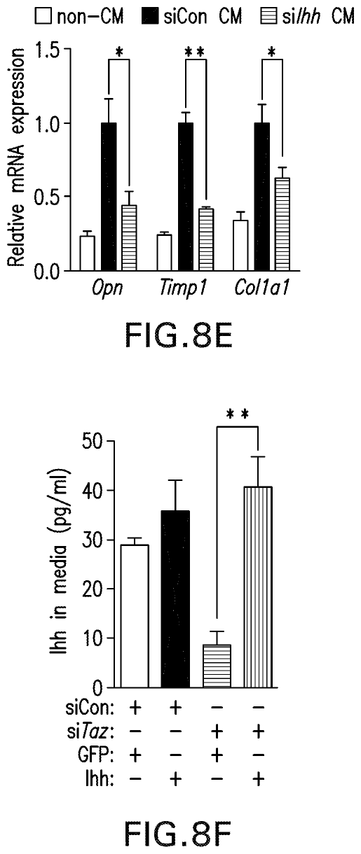

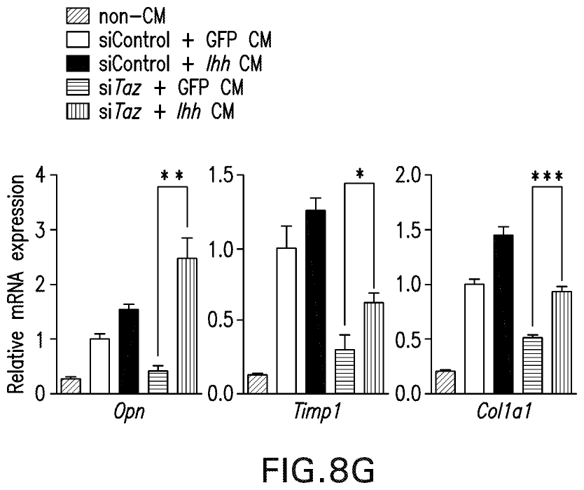

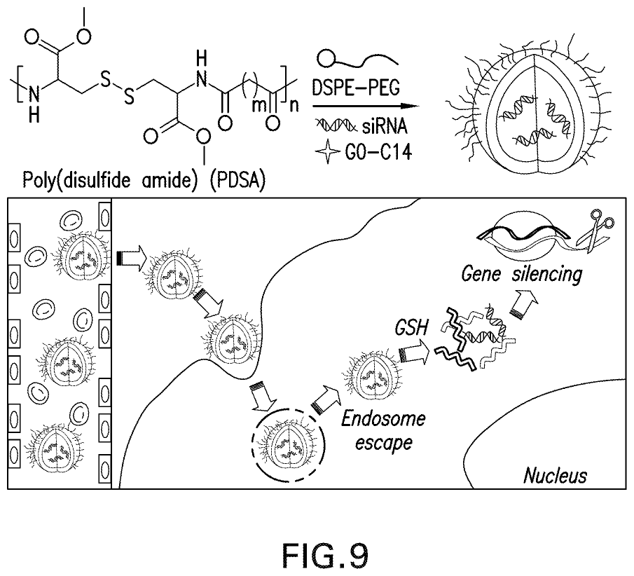

To explore the possibility that TAZ-induced Ihh is secreted by hepatocytes and activates HSCs, an in vitro model using AML12 cells, a non-cancerous mouse hepatocyte cell line was utilized (Dumenco et al., 1995), along with primary murine HSCs. Consistent with the in vivo data, siTaz treatment of AML12 hepatocytes lowered cellular Ihh mRNA in the cells and Ihh protein in both the cells and media (FIGS. 8A-C). In the next experiments, conditioned medium (CM) from control or Taz-silenced AML12 cells was utilized, as well as medium not exposed to cells (non-CM), to primary murine hepatic stellate cells (HSCs). Compared with non-CM, hepatocyte CM markedly increased Opn mRNA as well as the mRNAs for two proteins involved in fibrosis, Timp1 and Col1a1. Most importantly, CM from TAZ-silenced hepatocytes lowered the levels these mRNAs and protein compared to CM from shCon-treated hepatocytes (FIG. 8D). Conditioned medium from Ihh-silenced hepatocytes also decreased the three mRNAs in HSCs (FIG. 8E), although the absolute degree of Timp1 lowering was somewhat greater in this experiment than in the siTaz experiment. Finally, to make a more direct link between TAZ-induced Ihh in hepatocytes and activation of HSCs, Ihh was restored in siTaz-treated hepatocytes by Ihh transfection and then analyzed to determine whether this lessened the suppressive effect of CM from these cells on the expression of the fibrosis-related genes in HSCs. Transfection of TAZ-silenced hepatocytes with Ihh led to a level of Ihh in the CM that was similar to that in the CM of control hepatocytes (FIG. 8F-compare 1st and 4th bars). As before, the CM of TAZ-silenced hepatocytes suppressed Opn, Timp1 and Col1a1 mRNA in HSCs (FIG. 8G-compare 2nd and 4th bars), and the present results illustrate that restoration of Ihh in these TAZ-silenced cells rescued CM-induced HSC gene expression (FIG. 8G-compare 4th and 5th bars). Together, these studies suggest that hepatocyte TAZ-induced Ihh promotes the expression of pro-fibrotic genes, thus linking TAZ-IHH to the progression from benign steatosis to NASH and the development of liver fibrosis, a key determinant of outcome in patients with NASH.

### Discussion

NASH, characterized by inflammation, cell death, and fibrosis can progress to advanced liver disease, cirrhosis, and the need for liver transplant. Steatosis alone is believed to be a little to no risk for progressive liver disease. Given the clinical significance of NASH compared to steatosis (Rinella, 2015), an important objective of research in this area is to identify factors and pathways that promote the conversion of steatosis to NASH and the development of fibrosis. The importance of this objective is underscored by the fact that NASH is becoming the leading cause of liver disease worldwide and yet lacks any definitive, evidence-based drug therapies approved by the US Food and Drug Administration (Rinella, 2015). In this context, the finding that TAZ plays a key role in steatosis-to-NASH conversion and the development of fibrosis provides new insight into NASH and may suggest new targets for therapy.

Hepatic fibrosis is a key feature of NASH that distinguishes it from steatosis and determines long-term mortality in patients with NASH (Angulo et al., 2015). While both TAZ and YAP have been implicated in organ fibrosis in other settings, particularly in the lung with links to TGFα-SMAD signaling or induction of plasminogen activator inhibitor-1 (Liu et al., 2015; Mitani et al., 2009; Piersma et al., 2015; Saito and Nagase, 2015), there are only scattered reports about their roles in liver fibrosis, and none in the setting of NAFLD. For example, a recent study reported an association between microRNA-130/301, which can regulate TAZ and YAP, and carbon tetrachloride-induced liver fibrosis (Bertero et al., 2015), but there were no direct causation or mechanistic data related to the role of TAZ in this process. Another report showed that knockout of a pair of Hippo factors called Mps One Binder Kinase Activator (MOB)1A/1B in lean mice caused elevated TGFα-2/3 and liver fibrosis in a manner that was partially dependent on TAZ (Nishio et al., 2016).

Although the mechanism of TAZ in liver fibrosis in NASH is likely to be multifactorial, we provide evidence that the TAZ target Ihh may be important. Previous work has implicated hedgehog signaling in NASH fibrosis, particularly Shh signaling in HSCs (Bohinc and Diehl, 2012), both directly and via the induction of the pro-fibrotic cytokine IL-13 by immune cells (Shimamura et al., 2008; Syn et al., 2010). ChIP array data suggested that Ihh is a YAP/TAZ/TEAD target gene (Zhao et al., 2008), and using ChIP analysis of liver from FPC-fed mice, the present data illustrate for the first time that TAZ interacts with a highly conserved TAZ/TEAD consensus sequence in intron 1 of the Taz gene. Most importantly, hepatic Ihh was induced by the FPC diet and suppressed by shTaz. Moreover, the presently described in vitro study illustrated that hepatocyte TAZ-induced IHH activates a fibrosis program in HSCs. Further support for this aspect will benefit from additional mechanistic and in vivo causation studies, with the realization that the development of fibrosis during NASH progression is complex and multi-factorial and that TAZ likely has actions in addition to inducing Ihh that contribute to NASH fibrosis.

Two other important features of NASH, inflammation and cell death, were also ameliorated by TAZ silencing as shown in the present results. Little is known about the pro-inflammatory roles of TAZ, and, in general, YAP and TAZ inhibit rather than promote apoptosis during development and in cancer (Yu et al., 2015). However, there is one report showing that siTaz decreased TNFα-induced apoptosis in salivary gland epithelial cells (Hwang et al., 2014). RIP3-mediated necroptosis may also be important in hepatocyte death in NASH (Gautheron et al., 2014), and therefore it is possible that TAZ promotes this pathway. Given the various consequences of cell necrosis, this action of TAZ could contribute to inflammation and fibrosis as well as cell death in NASH (Chan et al., 2015; Luedde et al., 2014).

While the present studies focused on TAZ in hepatocytes, it is also known that YAP is significantly increased in progenitor-like reactive-appearing ductular cells (RDCs) in human and mouse NASH liver (Machado et al., 2015a). We have also found increased YAP in the livers of FPC mice, and most of the YAP-positive cells did not co-localize with HNF4α-positive hepatocytes. While the role of YAP in NASH remains to be elucidated, there are correlations among YAP+ RDCs, fibrosis, accumulation of myofibroblasts, and expression of Shh and Opn. Of note, silencing TAZ in the livers of FPC mice did not affect YAP expression. It is expected that inhibiting YAP or any one or more of the YAP co-factors TEAD1-4 may also provide useful therapeutic effects in the context of inhibiting, preventing, or treating NASH and NASH related conditions.

The majority of the present experiments were conducted in a mouse model of insulin resistance and NAFLD that was a modification of previously described models (Charlton et al., 2011; Kohli et al., 2010). The NAFLD diet was based on human dietary risk factors for NASH, and the key improvement over previous models was the development of a high level of inflammation, hepatocyte death, and fibrosis in 16 weeks without the need for genetically engineered mutations and in the background of weight gain and insulin resistance. Whereas the fructose component of the diet likely contributes to steatosis (Abdelmalek et al., 2010; Ishimoto et al., 2013), the cholesterol and palmitic acid components may be important in NASH progression and perhaps TAZ induction. For example, the accumulation of unesterified cholesterol in the liver has been implicated in the development of NASH in humans (Ioannou, 2016) and in various mouse models (Subramanian et al., 2011; Van Rooyen et al., 2011; Wouters et al., 2008). The mechanisms(s) linking cholesterol to NASH are likely to be multifactorial. For example, studies using mice fed high-cholesterol diets have suggested that cholesterol can directly activate HSCs by inducing TLR4, promote oxidative stress and cell death in hepatocytes via excess mitochondrial cholesterol, and promote inflammation in Kupffer cells through lysosomal cholesterol enrichment (Bieghs et al., 2013; Rawson, 2006; Teratani et al., 2012). How cholesterol links to the TAZ pathway described in this report will be an important topic of future investigation. Likewise, palmitic acid has been reported to induce pro-inflammatory cytokine production by hepatocytes and Kupffer cells during NASH (Joshi-Barve et al., 2007; Miura et al., 2013), but other mechanisms may be involved as well. Thus, we would expect that lowering palmitic acid and/or lowering or decreasing unesterified cholesterol in the liver would have a beneficial effect on NASH and could be utilized in combination with any of the methods described herein. Finally, these studies illustrated that lowering the normally very high levels of vitamin E in previous murine diets, improved the model with respect to duplicating the human NASH features, in line with the modest protective effects of vitamin E in human NASH.

In summary, the present data shows that the Hippo pathway transcription factor TAZ is elevated in the livers of humans with NASH, which is recapitulated in mouse models. In these models, silencing of TAZ suppresses key features of NASH progression but not steatosis. These data provide new insight into the pathophysiology of NASH and raise the prospect of liver-directed TAZ inhibition as a new therapeutic strategy to prevent NASH progression.

### EXPERIMENTAL PROCEDURES

### Reagents and Antibodies

The following antibodies were used for immunoblots: GAPDH (#3683), β-actin (#5125), CHOP (#5554), TAZ (#8418), p-eIF2α (#3398), eIF2α (#5324), Lamin A/C (#4777) from Cell Signaling; p-TAZ (sc-17610) and Col1a1 (sc-8784) from Santa Cruz; Ihh (ab39634) from Abcam; and Timp1 (AF980) from R & D. The following antibodies were using for immunostaining of liver: α-SMA (ACTA2) (C6198, F3777) and TAZ (HPA007415) from Sigma; F4/80 (MCA497GA) from AbD Serotec; Ly6g (#127601) from Biolegend; Ly6b (MCA771G) from Bio-Rad; OPN (AF808) from R & D; 4-hydroxynonenal (4-HNE) (AB5605) from Millipore; and HNF4α (sc-6556) from Santa Cruz. The following plasma assay kits were used in this study: insulin ELISA (#90080) from Crystal Chem; MCP1 ELISA (#88-7391-22) from eBiosciences; cholesterol (#439-17501) and triglyceride (#465-09791, #461-09891) from Wako; and ALT (#006A-CR) and AST (#004A-CR) from BQ Kits, Inc. AAV8-shRNA targeting murine Taz was made by annealing complementary oligonucleotides (5′-CACCAcagccgaatctcgcaatgaatCTCGAGATTCATTGCGAG ATTCGGCTG-3′) (SEQ ID NO:1), which were then ligated into the pAAV-RSV-GFP-H1 vector, as described previously (Lisowski et al., 2014). The resultant constructs were amplified by the Salk Institute Gene Transfer, Targeting, and Therapeutics Core.

### Animal Studies

Male wild-type mice C57BL/6J (#000664, 8-10 weeks/old) and MC4R-negative loxTB Mc4r mice (#006414, 6 weeks/old), referred to here as Mc4r^{-/-} mice, were obtained from Jackson Laboratory (Bar Harbor, ME) and were allowed to adapt to housing in the Columbia University Medical Center Institute of Comparative Medicine for 1 week prior to random assignment to experimental cohorts. The mice were then fed the following diets for the times indicated in the figure legends: (a) chow diet (Picolab rodent diet 20, #5053); (b) “fast-food” (FF) diet (TestDiet 1810060): high-fat diet with drinking water containing 42 g/L glucose and fructose (55%/45%, w/w); or (c) fructose-palmitate-cholesterol (“FPC”) diet (Teklad, TD.140154): similar to FF diet but with 1.25% added cholesterol and with palmitic acid, anhydrous milk fat, and Primex as the sources of fat and with a ˜60% decrease in vitamin E and a ˜35% decrease in choline compared with typical mouse diets. The detailed composition of these diets is shown in Tables 1 and 2. For several experiments, groups of mice were placed on a methionine-choline-deficient diet (Teklad, TD. 90262) for 8 weeks, as described (Dixon et al., 2012). Adeno-associated virus (2×10¹¹ genome copy/mouse) was delivered by tail vein injection either 1 week prior to diet initiation or after 8 weeks of the FPC diet. Animals were housed in standard cages at 22° C. in a 12-12-h light-dark cycle. All animal experiments were performed in accordance with institutional guidelines and regulations and approved by the Institutional Animal Care and Use Committee at Columbia University.

### Human Samples

Liver biopsy specimens from individuals undergoing weight loss surgery were selected from the MGH NAFLD Biorepository. Patients gave informed consent at the time of recruitment, and their records were anonymized and de-identified. Studies were approved by the Partners Human Research Committee (IRB) and conducted in accordance with National Institutes of Health and institutional guidelines for human subject research. Additional anonymized and de-identified liver biopsy sections were obtained from Dr. Jay Lefkowitch, Columbia University Medical Center. Cases with NAFLD activity score (NAS) of 1-3 were classified as early NAFLD (no fibrosis), while cases with NAS>5 and fibrosis stage 1a/b-4 were classified as NASH. Cases with steatosis score >1 and inflammation and ballooning scores of 0 and no fibrosis were classified as steatosis. Cases with NAS 0 were classified as normal.

### Blood and Plasma Analyses

Fasting blood glucose was measured using a glucose meter (One Touch Ultra, Life-scan) in mice that were fasted for 4-5 h, with free access to water. Complete blood counts were obtained with the FORCYTE Veterinary Hematology Analyzer (Oxford Science, Inc.). Total plasma triglyceride and cholesterol were assayed using a commercially available kit from Wako. For insulin, MCP1, AST, ALT, TC, TG are measured following kit instruction by using plasma.

### Histopathological Analysis, Immunohistochemistry, and Immunofluorescence Microscopy

Formalin-fixed, paraffin-embedded liver sections were stained with hematoxylin and eosin (H&E) and evaluated for severity of NAFLD by a trained hepatopathologist blinded to the clinical diagnosis, αaccording to criteria described by Brunt et al. (Kleiner et al., 2005; Liang et al., 2014). Liver fibrosis was assessed by Picrosirius (Sirius) red (Polysciences, #24901) or by Masson's trichrome staining (Sigma, HT15), with aniline blue-positive areas quantified as a measure of collagen content in the trichrome-stained sections. TUNEL staining was conducted using a kit from Roche (#12156792910). For immunofluorescence microscsopy, paraffin sections were rehydrated, subjected to antigen retrieval by placing in a pressure cooker for 10 mins in Target Retrieval Solution (Dako, S1699), and then blocked with serum. Sections were labeled with primary antibodies overnight, using a 1:150 dilution except for α-SMA and 4-HNE (1:200) and TAZ (1:400), followed by incubation with a fluorophore-conjugated secondary antibody for 1 h. The stained sections were mounted with DAPI-containing mounting medium (Life Technologies, P36935) and then viewed on an Olympus IX 70 fluorescence microscope. For filipin (Sigma, F9765) staining, frozen sections were fixed in 4% paraformaldehyde for 1 h at room temperature, then rinsed using glycine/PBS and stained 0.25 mg/ml filipin 2 h at room temperature. Fluorescence microscopic images were analyzed using ImageJ software. For immunohistochemistry, the deparaffinization, rehydration, and antigen retrieval processes were the same as with immunofluorescence staining. The slides were treated with 3% hydrogen peroxide for 10 min and then blocked with Serum-Free Protein Block (Dako, X0909) for 30 min. Sections were incubated with OPN, F4/80, or α-SMA primary antibody (1:100) overnight and then developed using DAB substrate kit (Cell Signaling, #8059) for OPN and F4/80, FITC-labeled anti-HRP secondary antibody for α-SMA.

### Measurement and Analysis of Liver Tissue Fatty Acids and Cholesterol

Liver specimens (˜20 mg) were homogenized in 600 μl of 5% ethanol, and then 6 μl was added to 100 μl KOH (1M, 9:1 methanol:H₂O). The suspension was heated at 100° C. for 30 min and then clarified by centrifugation, followed by addition of 80 μl HCl to the supernate. Fatty acids in this solution were identified and quantified by gas chromatography in the Columbia Biomarker Core Laboratory. For liver cholesterol quantification, liver tissue was homogenized in H₂O. Color Reagent Solution from the Wako Total Cholesterol assay kit was added at a 1:20 ratio (v/v) to the liver lysates. The suspension was then centrifuged, and the supernates were read in a plate reader.

### Immunoblotting

Liver protein was extracted using RIPA buffer (Thermo, #89900), and the protein concentration was measured by a BCA assay (Thermo, #23227). Proteins were separated by electrophoresis on 4-20% Tris gels (Life technologies, EC60285) and transferred to a nitrocellulose membranes (Bio-Rad, #1620115). The membranes were blocked for 30 min at room temperature in Tris-buffered saline and 0.1% Tween 20 (TBST) containing 5% (wt/vol) nonfat milk and then incubated with primary antibody in the same buffer at 4° C. overnight, using 1:1000 dilution except for CHOP and Ihh (1:3000). The protein bands were detected with horse radish peroxidase-conjugated secondary antibodies (Cell Signaling) and Supersignal West Pico enhanced chemiluminescent solution (Thermo, #34080). Cultured cells were lysed in Laemmli sample buffer (Bio-Rad, #161-0737) containing 5% 2-mercaptoethanol, heated at 100° C. for 5 min, and then electrophoresed and immunoblotted as above. Preparation of nuclear and cytoplasmic fractions of liver was carried out using Nuclear Extract Kit (Active Motif, #40010) according to the manufacturer's protocol.

### Cell Culture

AML12 mouse hepatocytes were purchased from ATCC (CRL-2254) and cultured in DMEM/F12 medium (Lifetechnologies, #11320) with 10% FBS (Gibco, #16140-071). Hepatic stellate cells (HSCs) were isolated from 5-6 mo/o BALB/C mice as described previously (Mederacke et al., 2015). Briefly, after cannulation of the inferior vena cava, the portal vein was cut, allowing retrograde step-wise perfusion with solutions containing protease (Sigma Aldrich, P5147) and collagenase D (Roche, #11088866001). The perfusates were subjected to 9.7% Nycodenz (Accurate Chemical, #1002424) gradient centrifugation to isolate the HSCs, which were then plated in tissue culture dishes and used the next day. For conditioned medium transfer experiments, AML12 cells were cultured in DMEM containing 0.2% BSA and incubated for 24 h. The media were then transferred to HSCs that had previously been incubated in DMEM, 0.2% BSA for 24 h. After 72 h, the HSCs were assayed for gene expression. For quantification of Ihh, hepatocyte conditioned medium was concentrated 10-fold by centrifugal filters (Millipore, Ultracel) and analyzed by an ELISA kit (LifeSpan Biosciences, F7953).

### Quantitative RT-qPCR

Total RNA was extracted from liver tissue or primary cultured hepatocytes using the RNeasy kit (Qiagen, 74106). cDNA was synthesized from 1 μg total RNA using oligo (dT) and Superscript II (Invitrogen). qPCR was performed in an 7500 Real time PCR system (Applied Biosystems) using SYBR green chemistry (Life Technologies, #4367659). The primer sequences are listed in Table 4.

siRNA-Mediated Gene Silencing and Transfection

siRNA sequences against mouse Taz and scrambled RNA were purchased from IDT; the target sequence of Taz siRNA was ACA UGG ACG AGA UGG AUA CAG GUG A (SEQ ID NO:2). The scrambled RNA and siRNA were transfected into AML12 cells (ATCC) using RNAiMAX (Life Technologies, #13778150) according to the manufacturer's instruction. A plasmid encoding GFP was purchased from Lonza (pmaxGFP), and a plasmid encoding murine Ihh was purchased from Origene (MR227435). The plasmids were transfected into AML12 cells using Lipofectamine® LTX Reagent with PLUS™ Reagent (Life Technologies, #15338100).

### Mouse Liver Nuclei Preparation and ChIP Assays

Mouse liver tissues were homogenized using a Dounce homogenizer (Wheaton, #357544) with a loose pestle in 1:10 (w:v) of ice-cold NP-40 lysis buffer supplemented with a protease inhibitor cocktail. The release of nuclei from the homogenate was monitored by DAPI staining and fluorescence microscopy. To purify intact nuclei, lysates were layered over a step gradient consisting of 1 M and 0.68 M sucrose and then centrifuged at 4000 rpm for 30 min at 4° C. Following a washing step, nuclear pellets were cross-linked with 1% fresh formaldehyde in PBS for 10 min at room temperature. Cross-linking was terminated by addition of 200 mM Tris-HCl (pH 9.4) and 1 mM DTT, and after 10 mins the suspension was centrifuged at 2500 rpm for 15 min at 4° C. Nuclear pellets were suspended in SDS lysis buffer containing protease inhibitors, incubated for 10 min on ice. DNA was sheared in a cold water bath using a focused-ultrasonicator (Covaris, S2) to obtain DNA fragments with an average size of 500 bps. Fragmented chromatin was pre-cleaned by incubating with normal rabbit IgG (Santa Cruz, sc-2027) for 1 h at 4° C., followed by 1 h of incubation with 50 μL protein G magnetic beads (Pierce, #88847) at 4° C. with rotation. Immunoprecipitation was conducted using a rabbit anti-TAZ antibody (Cell Signaling, #4883), and a control rabbit anti-HA antibody (Santa Cruz, sc-805) was used as a negative control. Immunoprecipitated chromatin fragments were reverse cross-linked, digested by proteinase K, and purified using QIAquick PCR Purification Kit (Qiagen, #28106). The presence of TAZ in Ihh intronic region was quantified by qPCR and expressed relative to the input genomic DNA. The sequences of primers used for the ChIP-qPCR assays, including negative control primers, are described in Table 5.

TABLE 5
Primers used for qPCR.

| Primers | Sequences |
|---|---|
| Hprt F | TCAGTCAACGGGGGACATAAA |
| | (SEQ ID NO: 3) |
| Hprt R | GGGGCTGTACTGCTTAACCAG |
| | (SEQ ID NO: 4) |
| Taz (Wwtr1) F | CATGGCGGAAAAAGATCCTCC |
| | (SEQ ID NO: 5) |
| Taz (Wwtr1) R | GTCGGTCACGTCATAGGACTG |
| | (SEQ ID NO: 6) |
| Tgfb1 F | CTCCCGTGGCTTCTAGTGC |
| | (SEQ ID NO: 7) |
| Tgfb1 R | GCCTTAGTTTGGACAGGATCTG |
| | (SEQ ID NO: 8) |
| Acta2 F | ATGCTCCCAGGGCTGTTTTCCCAT |
| | (SEQ ID NO: 9) |
| Acta2 R | GTGGTGCCAGATCTTTTCCATGTCG |
| | (SEQ ID NO: 10) |
| Vim F | TTTCTCTGCCTCTGCCAAC |
| | (SEQ ID NO: 11) |
| Vim R | TCTCATTGATCACCTGTCCATC |
| | (SEQ ID NO: 12) |
| Des F | CTAAAGGATGAGATGGCCCG |
| | (SEQ ID NO: 13) |
| Des R | GAAGGTCTGGATAGGAAGGTTG |
| | (SEQ ID NO: 14) |
| Collal F | GCTCCTCTTAGGGGCCACT |
| | (SEQ ID NO: 15) |
| Collal R | CCACGTCTCACCATTGGGG |
| | (SEQ ID NO: 16) |
| Colla2 F | GTAACTTCGTGCCTAGCAACA |
| | (SEQ ID NO: 17) |
| Colla2 R | CCTTTGTCAGAATACTGAGCAGC |
| | (SEQ ID NO: 18) |
| Col3al F | CTGTAACATGGAAACTGGGGAAA |
| | (SEQ ID NO: 19) |
| Col3al R | CCATAGCTGAACTGAAAACCACC |
| | (SEQ ID NO: 20) |
| F4/80 (Adgre1) F | ACCACAATACCTACATGCACC |
| | (SEQ ID NO: 21) |
| F4/80 (Adgre1) R | AAGCAGGCGAGGAAAAGATAG |
| | (SEQ ID NO: 22) |
| Tnfa F | CTTCTGTCTACTGAACTTCGGG |
| | (SEQ ID NO: 23) |
| Tnfa R | CAGGCTTGTCACTCGAATTTTG |
| | (SEQ ID NO: 24) |
| Mcp1 F | TTAAAAACCTGGATCGGAACCAA |
| | (SEQ ID NO: 25) |
| Mcp1 R | GCATTAGCTTCAGATTTACGGGT |
| | (SEQ ID NO: 26) |
| Ihh F | CTCTTGCCTACAAGCAGTTCA |
| | (SEQ ID NO: 27) |
| Ihh R | CCGTGTTCTCCTCGTCCTT |
| | (SEQ ID NO: 28) |
| Gli2 F | CAACGCCTACTCTCCCAGAC |
| | (SEQ ID NO: 29) |
| Gli2 R | GAGCCTTGATGTACTGTACCAC |
| | (SEQ ID NO: 30) |
| Gli3 F | CACAGCTCTACGGCGACTG |
| | (SEQ ID NO: 31) |
| Gli3 R | CTGCATAGTGATTGCGTTTCTTC |
| | (SEQ ID NO: 32) |
| Opn F | CTGACCCATCTCAGAAGCAGAATCT |
| | (SEQ ID NO: 33) |
| Opn R | TCCATGTGGTCATGGCTTTCATTGG |
| | (SEQ ID NO: 34) |
| Timp1 F | CTCAAAGACCTATAGTGCTGGC |
| | (SEQ ID NO: 35) |
| Timp1 R | CAAAGTGACGGCTCTGGTAG |
| | (SEQ ID NO: 36) |
| Cpt1b F | GCACACCAGGCAGTAGCTTT |
| | (SEQ ID NO: 37) |
| Cpt1b R | CAGGAGTTGATTCCAGACAGGTA |
| | (SEQ ID NO: 38) |
| Pparg F | TCGCTGATGCACTGCCTATG |
| | (SEQ ID NO: 39) |
| Pparg R | GAGAGGTCCACAGAGCTGATT |
| | (SEQ ID NO: 40) |
| Scdl F | CTGACCTGAAAGCCGAGAAG |
| | (SEQ ID NO: 41) |
| Scd1 R | AGAAGGTGCTAACGAACAGG |
| | (SEQ ID NO: 42) |
| Fasn F | AAGTCCCAGAAATCGCCTATG |
| | (SEQ ID NO: 43) |
| Fasn R | GGTATGGTTTCACGACTGGAG |
| | (SEQ ID NO: 44) |
| Acaca F | ATGGGCGGAATGGTCTCTTTC |
| | (SEQ ID NO: 45) |
| Acaca R | TGGGGACCTTGTCTTCATCAT |
| | (SEQ ID NO: 46) |
| Cd3 F | ATGCGGTGGAACACTTTCTGG |
| | (SEQ ID NO: 47) |
| Cd3 R | GCACGTCAACTCTACACTGGT |
| | (SEQ ID NO: 48) |
| Cd20 F | AACCTGCTCCAAAAGTGAACC |
| | (SEQ ID NO: 49) |
| Cd20 R | CCCAGGGTAATATGGAAGAGGC |
| | (SEQ ID NO: 50) |
| Ihh intron 1F | CAATCATTGACAGCGAGGGC |
| | (SEQ ID NO: 51) |
| Ihh intron 1F | GGTGTAGCTCGGTTCTGGTAG |
| | (SEQ ID NO: 52) |
| Ihh non-specific F | GGTGTAGCTCGGTTCTGGTAG |
| | (SEQ ID NO: 53) |
| Ihh non-specific R | TCACCTGGGACTCCATTTGC |
| | (SEQ ID NO: 54) |
| hTAZ F | TCCCAGCCAAATCTCGTGATG |
| | (SEQ ID NO: 72) |
| hTAZ R | AGCGCATTGGGCATACTCAT |
| | (SEQ ID NO: 81) |

### Abbreviations

HSCs: hepatic stellate cells. IHC: immunohistochemistry. NASH: nonalcoholic steatohepatitis. NAFLD: Non-alcoholic fatty liver disease. Hprt: hypoxanthineguanine phosphoribosyltransferase. Taz(Wwtr1): WW domain containing transcription regulator 1, encoding the TAZ protein (Reference human nucleotide sequence: NM_015472; Reference human protein sequence: NP_056287). Tgfp1: transforming growth factor, beta 1. Acta2, α-smooth muscle actin. Vim: vimentin. Des: desmin. Col1a1: collagen type I alpha 1. Col1a2: collagen type I alpha 2. Col3a1: collagen, type III, alpha 1. F4/80 (Adgre1): adhesion G protein-coupled receptor E1. Tnfa: tumor necrosis factor alpha. Mcp1: monocyte chemoattractant protein-1. Ihh: Indian hedgehog. Gli2: GLI family zinc finger 2. Gli3: GLI family zinc finger 3. Opn: osteopontin. Timp1: tissue inhibitor of metalloproteinase 1. Cpt1b: carnitine palmitoyltransferase 1B. Pparg: peroxisome proliferator-activated receptor-γ. Scd1: stearoyl-CoA desaturase. Fasn: fatty acid synthase. Acaca: acetyl-CoA carboxylase-α. Cd3: CD3 antigen. Cd20: B-lymphocyte antigen. Ihh intron: specific TAZ/TEAD binding area in 1^{st} intron of Ihh gene. Ihh non-specific: non-specific TAZ/TEAD binding site in mouse Ihh gene distal promoter. TEA: transcriptional enhancer factor. TEAD1: TEA Domain Family Member 1 (also known as SV40 Transcriptional Enhancer Factor or TEF-1), (Reference human nucleotide sequence: NM_021961; Reference human protein sequence: NP_068780). TEAD2: Transcriptional enhancer factor TEF-4 also known as TEA domain family member 2. (Reference human nucleotide sequence: NM_001256658; Reference human protein sequence: NP_001243587). TEAD3: TEA domain transcription factor 3 encodes transcriptional enhancer factor TEF-5. (Reference human nucleotide sequence: NM_003214; Reference human protein sequence: NP_003205). TEAD4: TEA Domain Family Member 4; (Reference human nucleotide sequence: NM_003213; Reference human protein sequence: NP_003204). YAP: Yes-associated protein 1, the gene encoding this protein is known as YAP1 or YAP65 (Reference human nucleotide sequence:_NM_001282101; Reference human protein sequences: UniProtKB/Swiss-Prot P46937.2 or NCB1 NP_001 123617.1).

### Measurement of Hydroxyproline Content of Liver Tissue

Hydroxyproline liver content was measured as previously described (Bataller et al., 2003; Seki et al., 2009). Briefly, liver tissue was homogenized, and proteins were precipitated using trichloroacetic acid. Samples were hydrolyzed by incubation with 6N hydrochloric acid at 110° C. for 16 h followed by neutralization with sodium hydroxide. Liver hydrolysates were oxidized using chloramine-T, followed by incubation with Ehrlich's perchloric acid reagent for color development. Absorbance was measured at 560 nm, and hydroxyproline quantities were calculated by reference to standards processed in parallel. Results are expressed as ng per mg liver weight.

### Statistical Analysis

All results are presented as mean±SEM. P values were calculated using the Student's t-test for normally distributed data and the Mann-Whitney rank sum test for non-normally distributed data. One-way ANOVA with post-hoc Tukey test was used to evaluate differences among groups when 3 or more groups were analyzed.

Redox-Responsive Liver-Targeting Nanoparticle Platform for siRNA Delivery

### Synthesis of the L-Cystine-Based Poly(Disulfide) (PDSA) Polymers

PDSA polymers were prepared by one-step polycondensation of L-cystine dimethyl ester dihydrochloride ((H-Cys-OMe)2.2HCl) and dichlorides or Bis-nitrophenol esters of different fatty diacids. A standard synthesis procedure was carried out as follows: (H-Cys-OMe)2.2HCl (10 mmol) and triethylamine (15 mmol) were dissolved in 20.0 mL DMSO, then the dichloride of fatty acid (10 mmol) DMSO solution (10 mL) was added into the cystine mixture solution dropwise. The solution was stirred for 15 min to obtain a uniform mixture, precipitated twice in 250 mL of cold ethyl ether, and dried under reduced atmosphere. The final product was a yellow or brown yellow powder.

### Redox-Responsive Behavior of the PDSA Polymers

GPC analysis was used to study the redox-responsive behavior of the PDSA polymers. The polymer (1 mg) was dissolved in 2 mL of DMF/H₂O (9:1, V/V) and then GSH (6.2 mg, 0.02 mmol) was added to obtain a solution with GSH concentration of 10 mM. At predetermined intervals, 100 μL of the solution was taken for GPC analysis.

### Preparation and Characterization of Nanoparticles (NPs)

The PDSA polymers were dissolved in DMF or DMSO to form a homogenous solution with a concentration of 20 mg/mL. Subsequently, 200 μL of this solution was taken and mixed with 140 μL of DSPE-PEG3000 (20 mg/mL in DMF), 50 μL of G0-C14 (5 mg/mL in DMF) and 1 nmol siRNA (0.1 nmol/μL aqueous solution). Under vigorously stirring (1000 rpm), the mixture was added dropwise to 5 mL of deionized water. The NP dispersion formed was transferred to an ultrafiltration device (EMD Millipore, MWCO 100 K) and centrifuged to remove the organic solvent and free compounds. After washing with deionized water (3×5 mL), the siRNA loaded NPs were dispersed in 1 mL of phosphate buffered saline (PBS, pH 7.4) solution. Size and zeta potential were determined by DLS. The morphology of NPs was visualized on TEM. To determine the siRNA encapsulation efficiency, DY547-labelled GL3 siRNA (DY547-siRNA) loaded NPs were prepared according to the method described above. A small volume (50 μL) of the NP solution was withdrawn and mixed with 20-fold DMSO. The fluorescence intensity of DY547-siRNA was measured using a Synergy HT multi-mode microplate reader (BioTek Instruments) and compared to the free DY-547 labelled GL3 siRNA solution (1 nmol/mL PBS solution).

### Redox-Responsive Behavior of the NPs

The siRNA loaded NPs were prepared as described above and dispersed in PBS containing 10 mM GSH. At pre-determined time point, the particle size was examined by DLS and the particle morphology was observed on TEM. To evaluate the intracellular redox-responsive behavior, the NPs with Nile red and coumarin 6 encapsulated in their hydrophobic cores were prepared and then incubated with HeLa cells for different time. The fluorescence of Nile red and coumarin 6 was observed a FV1000 confocal laser scanning microscope (CLSM, Olympus). If the NPs respond to redox stimulus, the Nile red and coumarin 6 will release and only green fluorescence of coumarin 6 can be observed under CLSM. If the NPs are intact, the fluorescence of coumarin 6 will be quenched by Nile red and only red fluorescence can be observed under CLSM.

### Evaluation of Endosomal Escape

Luc-HeLa cells (20,000 cells) were seeded in discs and incubated in 1 mL of RPMI 1640 medium containing 10% FBS for 24 h. Subsequently, the DY547-siRNA-loaded NPs were added, and the cells were allowed to incubate for 1 or 2 h. After removing the medium and subsequently washing with PBS (pH 7.4) solution thrice, the endosomes and nuclei were stained with lysotracker green and Hoechst 33342, respectively. The cells were then viewed under CLSM.

In Vitro siRNA Release

DY547-labelled siRNA (DY547-siRNA) was loaded into the NPs according to the method described above. Subsequently, the NPs were dispersed in 1 mL of PBS (pH 7.4) and then transferred to a Float-a-lyzer G2 dialysis device (MWCO 100 kDa, Spectrum) that was immersed in PBS (pH 7.4) at 37° C. At a predetermined interval, 5 μL of the NP solution was withdrawn and mixed with 20-fold DMSO. The fluorescence intensity of DY547-siRNA was determined by Synergy HT multi-mode microplate reader.

### Luciferase Silencing

Luciferase expressing HeLa (Luc-HeLa) cells were seeded in 96-well plates (5,000 cells per well) and incubated in 0.1 mL of RPMI 1640 medium with 10% FBS for 24 h. Thereafter, the Luc siRNA-loaded NPs were added. After incubating for 24 h, the cells were washed with fresh medium and allowed to incubate for another 48 h. The expression of firefly luciferase in HeLa cells was determined using Steady-Glo luciferase assay kits. Cytotoxicity was measured using the Alamar Blue assay according to the manufacturer's protocol. The luminescence or fluorescence intensity was measured using a microplate reader, and the average value of five independent experiments was collected. As a control, the silencing effect of Lipo2K/Luc siRNA complexes was also evaluated according to the procedure described above and compared to that of Luc siRNA loaded NPs.

Preparation of Wwtr1 (TAZ) siRNA Loaded NPs

The PDSA polymers were dissolved in DMF to form a homogenous solution with a concentration of 20 mg/mL. Subsequently, 200 μL of this solution was taken and mixed with 140 μL of DSPE-PEG3000 (20 mg/mL in DMF), 50 μL of G0-C14 (5 mg/mL in DMF) and 1 nmol TAZ siRNA (0.1 nmol/μL aqueous solution). Under vigorously stirring (1000 rpm), the mixture was added dropwise to 5 mL of deionized water. The NP dispersion formed was transferred to an ultrafiltration device (EMD Millipore, MWCO 100 K) and centrifuged to remove the organic solvent and free compounds. After washing with deionized water (3×5 mL), the TAZ siRNA loaded NPs were dispersed in 1 mL of phosphate buffered saline (PBS, pH 7.4) solution.

### In Vitro TAZ Silencing

Hepatocytes (AML12 cells) were seeded in 6-well plates (50,000 cells per well) and incubated in 1 mL of DMEM/F12 (1:1, v:v) medium containing 10% FBS for 24 h. Subsequently, the cells were incubated with the TAZ siRNA loaded NPs for 24 h. After washing the cells with PBS thrice, the cells were further incubated in fresh medium for another 48 h. Thereafter, the cells were digested by trypsin and the proteins were extracted using modified radioimmunoprecipitation assay lysis buffer (50 mM Tris-HCl pH 7.4, 150 mM NaCl, 1% NP-40 substitute, 0.25% sodium deoxycholate, 1 mM sodium fluoride, 1 mM Na3VO4, 1 mM EDTA), supplemented with protease inhibitor cocktail and 1 mM phenylmethanesulfonyl fluoride (PMSF). The expression of TAZ was examined using the western blot analysis.

### Western Blot

Equal amounts of proteins were added to SDS-PAGE gels and separated by gel electrophoresis. After transferring the proteins from gel to polyvinylidene difluoride (PVDF) membrane, the blots were blocked with 3% BSA in TBST (50 mM Tris-HCl pH 7.4, 150 mM NaCl, and 0.1% Tween 20) and then incubated with a mixture of TAZ rabbit antibody (Cell Signaling, catalog #8418S) and β-actin rabbit antibody (Cell Signaling, catalog #13E5). The expression of TAZ was detected with horseradish peroxidase (HRP)-conjugated secondary antibody (anti-rabbit IgG HRP-linked antibody, Cell Signaling) and an enhanced chemiluminescence (ECL) detection system (Pierce).

### Animals

Healthy female C57BL/6 mice (4-5 weeks old) were purchased from Charles River Laboratories. All in vivo studies were performed in accordance with National Institutes of Health animal care guidelines and in strict pathogen-free conditions in the animal facility of Brigham and Women's Hospital. Animal protocol was approved by the Institutional Animal Care and Use Committees on animal care (Harvard Medical School).

### Pharmacokinetics Study

Healthy female C57BL/6 mice were randomly divided into two groups (n=3) and given an intravenous injection of either (i) free DY647-labelled Luc siRNA (DY647-siRNA) or (ii) DY647-siRNA loaded NPs at a 1 nmol siRNA dose per mouse. At predetermined time intervals, orbital vein blood (20 μL) was withdrawn using a tube containing heparin, and the wound was pressed for several seconds to stop the bleeding. The fluorescence intensity of DY-647 labelled siRNA in the blood was determined using a microplate reader.

### Biodistribution

Healthy female C57BL/6 mice were randomly divided into two groups (n=3) and given an intravenous injection of either (i) free DY677-labelled Luc siRNA (DY677-siRNA) or (ii) DY677-siRNA loaded NPs at a 1 nmol siRNA dose per mouse. Twenty-four hours after the injection, the mice were imaged using the Maestro 2 In-Vivo Imaging System (Cri Inc). Main organs were then harvested and imaged. To quantify the accumulation of NPs in tumors and organs, the fluorescence intensity of each tissue was quantified by Image-J.

### Immune Response

Healthy female C57BL/6 mice were randomly divided into three groups (n=3) and given an intravenous injection of either (i) PBS, (ii) naked TAZ siRNA or (iii) TAZ siRNA loaded NPs at a 1 nmol siRNA dose per mouse. Twenty-four hours after injection, blood was collected and serum isolated for measurements of representative cytokines (TNF-α, IL-6, IL-12, and IFN-γ) by enzyme-linked immunosorbent assay or ELISA (PBL Biomedical Laboratories and BD Biosciences) according to the manufacturer's instructions.

### Histology

Healthy female C57BL/6 mice were randomly divided into three groups (n=3) and administered daily intravenous injections of either (i) PBS or (ii) TAZ siRNA loaded NPs at a 1 nmol siRNA dose per mouse. After three consecutive injections (once every day), the main organs were collected 24 h post the final injection, fixed with 4% paraformaldehyde, and embedded in paraffin. Tissue sections were stained with hematoxylin-eosin (H&E) and viewed under an optical microscope.

### Example 2 Design and Synthesis of Nanoparticles for siRNA Delivery to Target Liver Genes

Redox-responsive hydrophobic polymer was synthesized which could co-assemble with lipid-PEG to form spherical NPs for siRNA delivery to target liver genes involved in non-alcoholic steatohepatitis (NASH). The intracellular levels of glutathione (GSH) are much higher than that in extracellular fluid. Redox-sensitive approach is particularly promising to enhance the exposure of target cells to therapeutic molecules. In this example, L-cystine dimethyl ester and fatty diacid were used to synthesize a library of L-cystine-based poly(disulfide amide) polymers (PDSA).

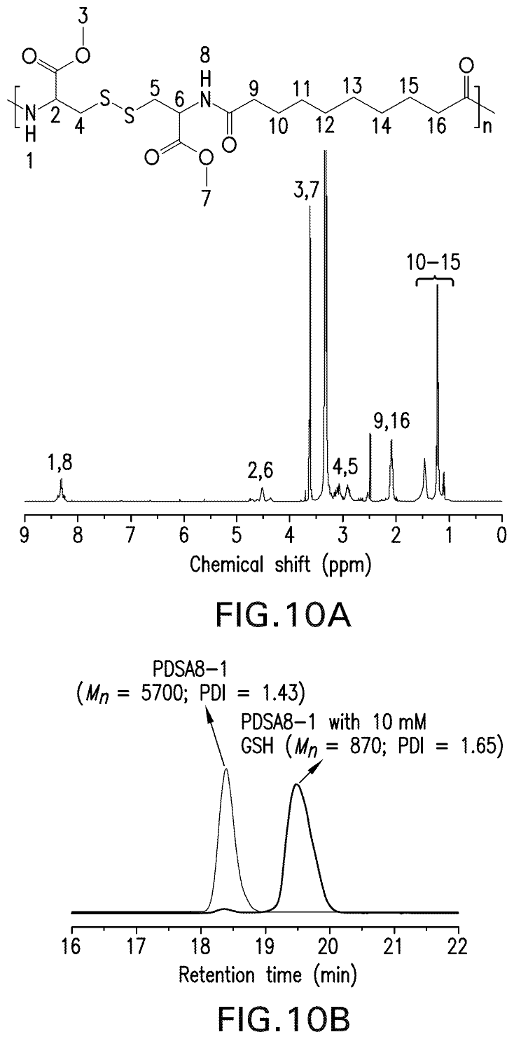

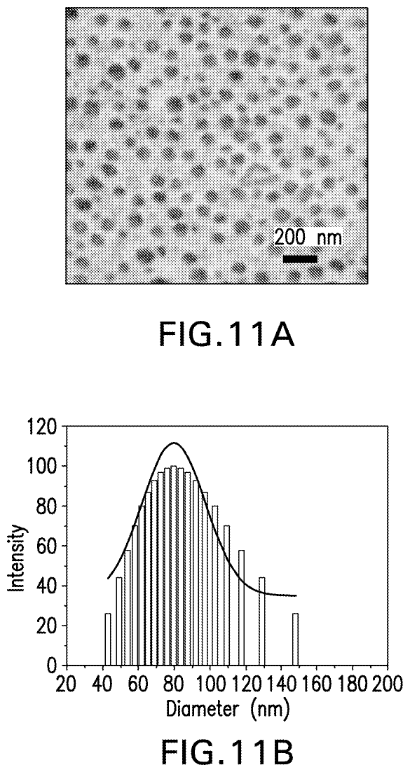

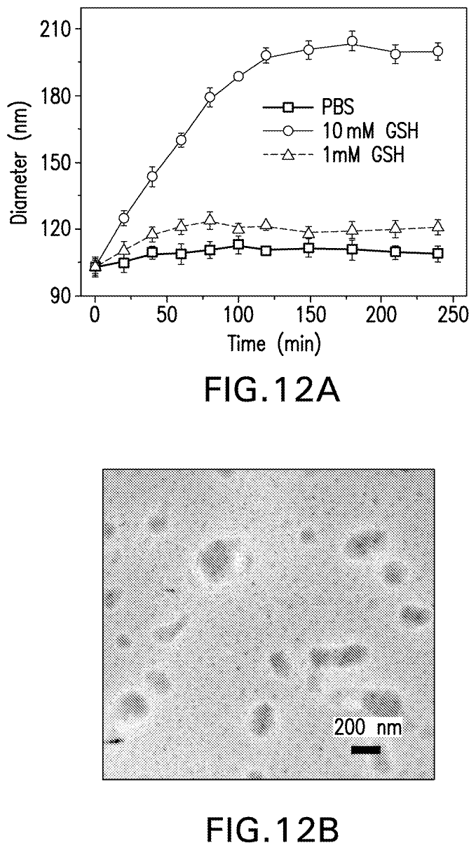

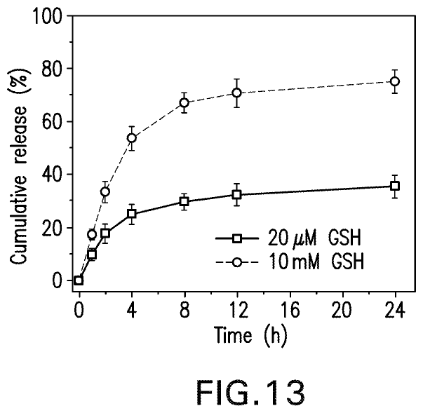

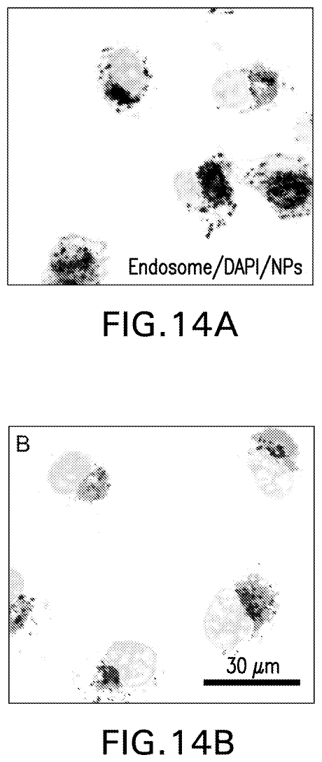

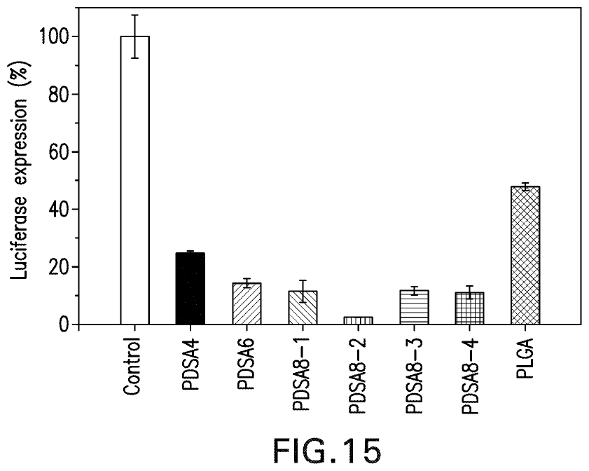

Feed compositions and molecular weight of the PDSA polymers are summarized in Table 6. Taking PDSA8-1, for example, the NMR spectrum in FIG. 10A demonstrates the success in the synthesis of this polymer. With the presence of multiple disulfide bonds, there is a significant decrease in the molecule weight of PDSA8-1 incubated in 10 mM glutathione (GSH) solution (FIG. 10B), demonstrating the redox response of the PDSA8-1 polymer. When mixing this redox-responsive polymer with DSPE-PEG3000, siRNA and cationic lipid (Xiaoyang Xu et al. Proc Natl Acad Sci USA, 2013, 110, 18638-18643) in water miscible solvent such as DMF, DMSO, etc., spherical NPs with an average size of ˜100 nm (FIG. 11) can be formed via nanoprecipitation method, in which hydrophilic PEG chains are on the outer shell and siRNA is encapsulated in the hydrophobic core. The physiochemical properties of the siRNA loaded NPs made with other PDSA polymers are summarized in Table 7. When incubating these siRNA loaded NPs (e.g., PDSA8-1 NPs, FIG. 12) with 10 mM GSH, the breakage of the disulfide bonds in the polymer backbone induces aggregation and increase in the particle size, which thereby leads to fast siRNA release (FIG. 13). In vitro experimental results show that the PDSA8-1 NPs have efficient endosomal escape ability as seen in fluorescent images of Luc-HeLa cells incubated with the siRNA loaded NPs for 1 and 4 h (FIG. 14). Moreover, with the highly concentrated GSH in cytoplasm to induce the breakage of the NPs, the siRNA loaded PDSA8-1 NPs show high efficacy in silencing Luc expression in HeLa cells (>90% knockdown at 10 nM siRNA dose, FIG. 15), which is much higher than that of cells treated with Luc siRNA loaded PLGA NPs (non-redox-responsive), demonstrating that the redox response predominately contributes to the efficient gene silencing of the PDSA8-1 NPs.

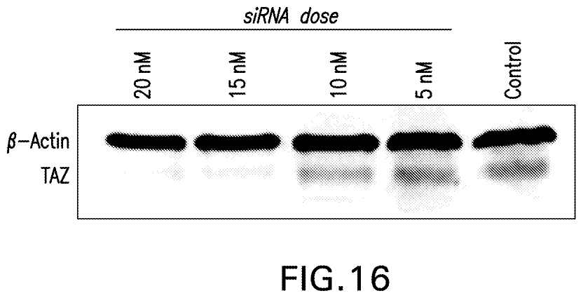

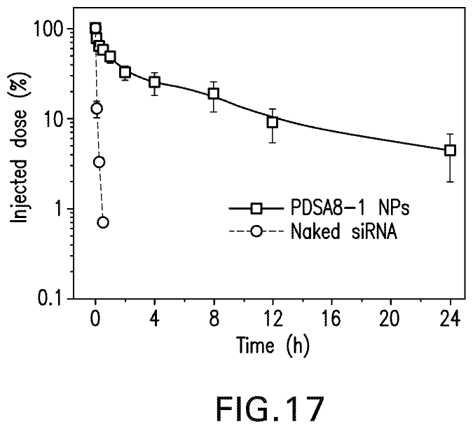

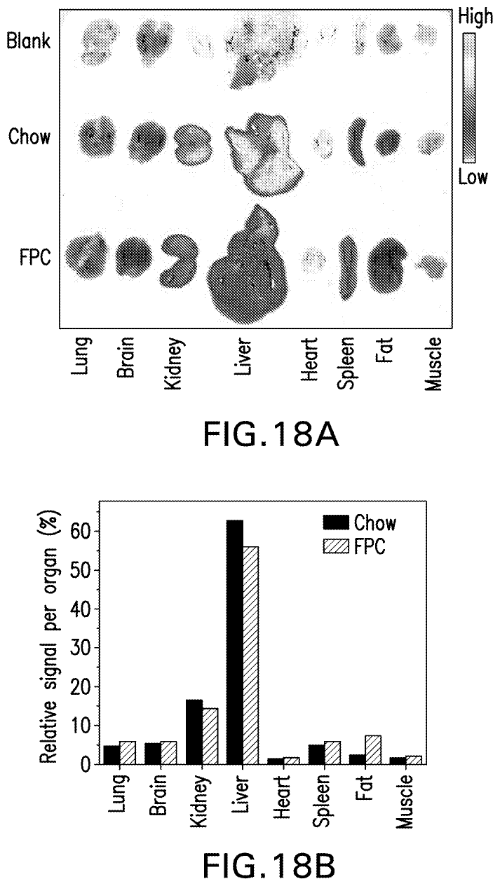

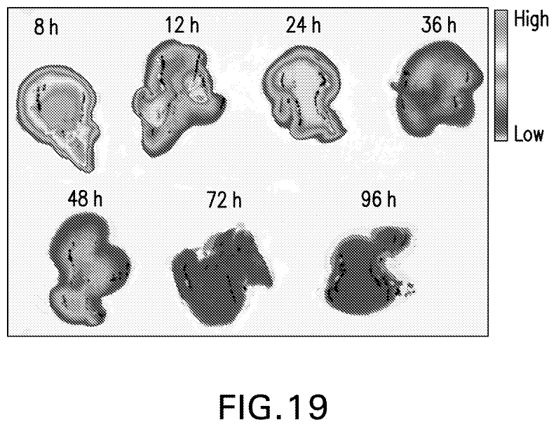

These redox-responsive NPs can be used as a robust nanoplatform to deliver therapeutic siRNA to target liver genes involved in NASH. After treatment with the NPs loaded TAZ siRNA, there is a significant decrease in the TAZ expression in hepatocytes, as demonstrated by the western blot result shown in FIG. 16. In vivo experimental results demonstrated that these NPs have a long/desirable blood circulation (FIG. 17) and show high targeted accumulation in liver tissue (FIGS. 18 and 19).

TABLE 6
Feed compositions and molecular weight of the PDSA polymers.

| | Poly(disulfide amide) | Mₙª | M_{w} | Polydispersity |
|---|---|---|---|---|
| m = 4 | Cys-PDSA4 | 2900 | 4300 | 1.48 |
| m = 6 | Cys-PDSA6 | 3900 | 5700 | 1.46 |
| m = 8 | Cys-PDSA8-1 | 4700 | 7800 | 1.66 |
| m = 8 | Cys-PDSA8-2 | 5700 | 7300 | 1.43 |
| m = 8 | Cys-PDSA8-3 | 9300 | 15200 | 1.63 |
| m = 8 | Cys-PDSA8-4 | 11700 | 16600 | 1.42 |

*Determined by GPC using DMF as the eluent

TABLE 7
Size, siRNA encapsulation efficiency (EE %) and zeta potential
of the NPs of PDSA polymers.

| | | | Cys- | Cys- | Cys- | Cys- |
|---|---|---|---|---|---|---|
| | Cys- | Cys- | PDSA8- | PDSA8- | PDSA8- | PDSA8- |
| | PDSA4 | PDSA6 | 1 | 2 | 3 | 4 |
| Size | 155.7 | 134.5 | 118.9 | 102.9 | 99.4 | 93.4 |
| (nm)ª | | | | | | |
| EE %^{b} | 29.7 | 35.1 | 46.3 | 55.9 | 79.4 | 88.2 |
| ξ (mV) | −6.79 | −8.08 | −9.79 | −11.21 | −12.05 | −20.01 |

^{a}N/P ratio is 20:1.
^{b}siRNA encapsulation efficiency.

Additional methods relating to nanoparticle siRNA formulations can be found in U.S. Patent Publication No. 20160022835. Desirable features of nanoparticle delivery of the TAZ siRNA and related inhibitors described herein include increased stability and the ability to avoid immune degradation. It is noted that the TAZ siRNA NPs have two main components: 1) a hydrophobic inner core that is made with redox-responsive polymers to encapsulate TAZ siRNA, and 2) a hydrophilic outer shell that can allow the TAZ NPs to evade recognition by immune system components and increase blood circulation half-life. The NPs may also include a third component: 3) a targeting ligand that can specifically bind to its receptor on hepatocytes. As demonstrated in the data described herein, the TAZ siRNA NPs exhibited the ability to knock down TAZ expression to a high degree.

A number of additional techniques will be suitable for liver-specific targeting of the present compositions including pharmaceutical compositions described herein. Such methods can be found in U.S. Patent Publication No. 20160017335 and Fitzgerald et al. (N. Engl. J. Med. 2017; 376:41-51; Jan. 5, 2017 DOI: 10.1056/NEJMoa1609243). These methods for producing modified siRNA have been applied to the proprotein convertase subtilisin-kexin type 9 (PCSK9) target and have produced stable siRNA's that have excellent, very low toxicity profiles and are undergoing clinical trials, as described for the product Inclisiran (ALN-PCSsc), a long-acting RNA interference (RNAi) therapeutic agent that inhibits the synthesis of PCSK9, a target for the lowering of low-density lipoprotein (LDL) cholesterol.

Such methods for producing a stable and modified siRNA of the TAZ and related inhibitors described herein would be expected to exhibit similarly desirable profiles.

### REFERENCES

Abdelmalek, M. F., Suzuki, A., Guy, C., Unalp-Arida, A., Colvin, R., Johnson, R. J., Diehl, A. M., and Nonalcoholic Steatohepatitis Clinical Research, N. (2010). Increased fructose consumption is associated with fibrosis severity in patients with nonalcoholic fatty liver disease. Hepatology 51, 1961-1971.
Angulo, P., Kleiner, D. E., Dam-Larsen, S., Adams, L. A., Bjornsson, E. S., Charatcharoenwitthaya, P., Mills, P. R., Keach, J. C., Lafferty, H. D., Stahler, A., et al. (2015). Liver Fibrosis, but No Other Histologic Features, Is Associated With Long-term Outcomes of Patients With Nonalcoholic Fatty Liver Disease. Gastroenterology 149, 389-397 e310.
Bataller, R., Schwabe, R. F., Choi, Y. H., Yang, L., Paik, Y. H., Lindquist, J., Qian, T., Schoonhoven, R., Hagedorn, C. H., Lemasters, J. J., et al. (2003). NADPH oxidase signal transduces angiotensin II in hepatic stellate cells and is critical in hepatic fibrosis. J Clin Invest 112, 1383-1394.
Bertero, T., Cottrill, K. A., Annis, S., Bhat, B., Gochuico, B. R., Osorio, J. C., Rosas, I., Haley, K. J., Corey, K. E., Chung, R. T., et al. (2015). A YAP/TAZ-miR-130/301 molecular circuit exerts systems-level control of fibrosis in a network of human diseases and physiologic conditions. Sci Rep 5, 18277.
Bieghs, V., Hendrikx, T., van Gorp, P. J., Verheyen, F., Guichot, Y. D., Walenbergh, S. M., Jeurissen, M. L., Gijbels, M., Rensen, S. S., Bast, A., et al. (2013). The cholesterol derivative 27-hydroxycholesterol reduces steatohepatitis in mice. Gastroenterology 144, 167-178 e161.
Bohinc, B. N., and Diehl, A. M. (2012). Mechanisms of disease progression in NASH: new paradigms. Clin Liver Dis 16, 549-565.
Chan, F. K., Luz, N. F., and Moriwaki, K. (2015). Programmed necrosis in the cross talk of cell death and inflammation. Annu Rev Immunol 33, 79-106.
Charlton, M., Krishnan, A., Viker, K., Sanderson, S., Cazanave, S., McConico, A., Masuoko, H., and Gores, G. (2011). Fast food diet mouse: novel small animal model of NASH with ballooning, progressive fibrosis, and high physiological fidelity to the human condition. Am J Physiol Gastrointest Liver Physiol 301, G825-834.
Clapper, J. R., Hendricks, M. D., Gu, G., Wittmer, C., Dolman, C. S., Herich, J., Athanacio, J., Villescaz, C., Ghosh, S. S., Heilig, J. S., et al. (2013). Diet-induced mouse model of fatty liver disease and nonalcoholic steatohepatitis reflecting clinical disease progression and methods of assessment. Am J Physiol Gastrointest Liver Physiol 305, G483-495.
Day, C. P., and James, O. F. (1998). Steatohepatitis: a tale of two “hits”? Gastroenterology 114, 842-845.
Dixon, L. J., Berk, M., Thapaliya, S., Papouchado, B. G., and Feldstein, A. E. (2012). Caspase-1-mediated regulation of fibrogenesis in diet-induced steatohepatitis. Lab Invest 92, 713-723.
Dumenco, L., Oguey, D., Wu, J., Messier, N., and Fausto, N. (1995). Introduction of a murine p53 mutation corresponding to human codon 249 into a murine hepatocyte cell line results in growth advantage, but not in transformation. Hepatology 22, 1279-1288.
Ekstedt, M., Franzen, L. E., Mathiesen, U. L., Thorelius, L., Holmqvist, M., Bodemar, G., and Kechagias, S. (2006). Long-term follow-up of patients with NAFLD and elevated liver enzymes. Hepatology 44, 865-873.
Friedman, S. L. (2008). Mechanisms of hepatic fibrogenesis. Gastroenterology 134, 1655-1669.
Ganz, M., Bukong, T. N., Csak, T., Saha, B., Park, J. K., Ambade, A., Kodys, K., and Szabo, G. (2015). Progression of non-alcoholic steatosis to steatohepatitis and fibrosis parallels cumulative accumulation of danger signals that promote inflammation and liver tumors in a high fat-cholesterol-sugar diet model in mice. J Transl Med 13, 193.
Gautheron, J., Vucur, M., Reisinger, F., Cardenas, D. V., Roderburg, C., Koppe, C., Kreggenwinkel, K., Schneider, A. T., Bartneck, M., Neumann, U. P., et al. (2014). A positive feedback loop between RIP3 and JNK controls non-alcoholic steatohepatitis. EMBO Mol Med 6, 1062-1074.
Hebbard, L., and George, J. (2011). Animal models of nonalcoholic fatty liver disease. Nat Rev Gastroenterol Hepatol 8, 35-44.
Hwang, S. M., Jin, M., Shin, Y. H., Ki Choi, S., Namkoong, E., Kim, M., Park, M. Y., and Park, K. (2014). Role of LPA and the Hippo pathway on apoptosis in salivary gland epithelial cells. Exp Mol Med 46, e125.
Ioannou, G. N. (2016). The Role of Cholesterol in the Pathogenesis of NASH. Trends Endocrinol Metab 27, 84-95.
Ishimoto, T., Lanaspa, M. A., Rivard, C. J., Roncal-Jimenez, C. A., Orlicky, D. J., Cicerchi, C., McMahan, R. H., Abdelmalek, M. F., Rosen, H. R., Jackman, M. R., et al. (2013). High-fat and high-sucrose (western) diet induces steatohepatitis that is dependent on fructokinase. Hepatology 58, 1632-1643.
Jolley, C. D., Dietschy, J. M., and Turley, S. D. (1999). Genetic differences in cholesterol absorption in 129/Sv and C57BL/6 mice: effect on cholesterol responsiveness. Am J Physiol 276, G1117-1124.
Joshi-Barve, S., Barve, S. S., Amancherla, K., Gobejishvili, L., Hill, D., Cave, M., Hote, P., and McClain, C. J. (2007). Palmitic acid induces production of proinflammatory cytokine interleukin-8 from hepatocytes. Hepatology 46, 823-830.
Kleiner, D. E., Brunt, E. M., Van Natta, M., Behling, C., Contos, M. J., Cummings, O. W., Ferrell, L. D., Liu, Y. C., Torbenson, M. S., Unalp-Arida, A., et al. (2005). Design and validation of a histological scoring system for nonalcoholic fatty liver disease. Hepatology 41, 1313-1321.
Kohli, R., Kirby, M., Xanthakos, S. A., Softic, S., Feldstein, A. E., Saxena, V., Tang, P. H., Miles, L., Miles, M. V., Balistreri, W. F., et al. (2010). High-fructose, medium chain trans fat diet induces liver fibrosis and elevates plasma coenzyme Q9 in a novel murine model of obesity and nonalcoholic steatohepatitis. Hepatology 52, 934-944.
Liang, W., Menke, A. L., Driessen, A., Koek, G. H., Lindeman, J. H., Stoop, R., Havekes, L. M., Kleemann, R., and van den Hoek, A. M. (2014). Establishment of a General NAFLD Scoring System for Rodent Models and Comparison to Human Liver Pathology. PloS one 9, e115922.
Lisowski, L., Dane, A. P., Chu, K., Zhang, Y., Cunningham, S. C., Wilson, E. M., Nygaard, S., Grompe, M., Alexander, I. E., and Kay, M. A. (2014). Selection and evaluation of clinically relevant AAV variants in a xenograft liver model. Nature 506, 382-386.
Liu, C. Y., Lv, X., Li, T., Xu, Y., Zhou, X., Zhao, S., Xiong, Y., Lei, Q. Y., and Guan, K. L. (2011). PP1 cooperates with ASPP2 to dephosphorylate and activate TAZ. J Biol Chem 286, 5558-5566.
Liu, F., Lagares, D., Choi, K. M., Stopfer, L., Marinkovic, A., Vrbanac, V., Probst, C. K., Hiemer, S. E., Sisson, T. H., Horowitz, J. C., et al. (2015). Mechanosignaling through YAP and TAZ drives fibroblast activation and fibrosis. Am J Physiol Lung Cell Mol Physiol 308, L344-357.
Luedde, T., Kaplowitz, N., and Schwabe, R. F. (2014). Cell death and cell death responses in liver disease: mechanisms and clinical relevance. Gastroenterology 147, 765-783 e764.
Machado, M. V., Michelotti, G. A., Pereira, T. A., Xie, G., Premont, R., Cortez-Pinto, H., and Diehl, A. M. (2015a). Accumulation of duct cells with activated YAP parallels fibrosis progression in non-alcoholic fatty liver disease. J Hepatol.
Machado, M. V., Michelotti, G. A., Xie, G., Almeida Pereira, T., Boursier, J., Bohnic, B., Guy, C. D., and Diehl, A. M. (2015b). Mouse models of diet-induced nonalcoholic steatohepatitis reproduce the heterogeneity of the human disease. PLoS One 10, e0127991.
McCullough, A. J. (2004). The clinical features, diagnosis and natural history of nonalcoholic fatty liver disease. Clin Liver Dis 8, 521-533, viii.
Mederacke, I., Dapito, D. H., Affo, S., Uchinami, H., and Schwabe, R. F. (2015). High-yield and high-purity isolation of hepatic stellate cells from normal and fibrotic mouse livers. Nat Protoc 10, 305-315.
Mederacke, I., Hsu, C. C., Troeger, J. S., Huebener, P., Mu, X., Dapito, D. H., Pradere, J. P., and Schwabe, R. F. (2013). Fate tracing reveals hepatic stellate cells as dominant contributors to liver fibrosis independent of its aetiology. Nat Commun 4, 2823.
Mitani, A., Nagase, T., Fukuchi, K., Aburatani, H., Makita, R., and Kurihara, H. (2009). Transcriptional coactivator with PDZ-binding motif is essential for normal alveolarization in mice. Am J Respir Crit Care Med 180, 326-338.
Miura, K., Yang, L., van Rooijen, N., Brenner, D. A., Ohnishi, H., and Seki, E. (2013). Toll-like receptor 2 and palmitic acid cooperatively contribute to the development of nonalcoholic steatohepatitis through inflammasome activation in mice. Hepatology 57, 577-589.
Nishio, M., Sugimachi, K., Goto, H., Wang, J., Morikawa, T., Miyachi, Y., Takano, Y., Hikasa, H., Itoh, T., Suzuki, S. O., et al. (2016). Dysregulated YAP1/TAZ and TGF-beta signaling mediate hepatocarcinogenesis in Mobla/1b-deficient mice. Proc Natl Acad Sci USA 113, E71-80.
Piersma, B., Bank, R. A., and Boersema, M. (2015). Signaling in Fibrosis: TGF-beta, WNT, and YAP/TAZ Converge. Front Med (Lausanne) 2, 59.
Rawson, R. B. (2006). An ARC light on lipid metabolism. Cell Metab 4, 181-183.
Razzaque, M. S., Soegiarto, D. W., Chang, D., Long, F., and Lanske, B. (2005). Conditional deletion of Indian hedgehog from collagen type 2alpha1-expressing cells results in abnormal endochondral bone formation. J Pathol 207, 453-461.
Rinella, M. E. (2015). Nonalcoholic fatty liver disease: a systematic review. JAMA 313, 2263-2273.
Saito, A., and Nagase, T. (2015). Hippo and TGF-beta interplay in the lung field. Am J Physiol Lung Cell Mol Physiol 309, L756-767.
Sanyal, A. J., Chalasani, N., Kowdley, K. V., McCullough, A., Diehl, A. M., Bass, N. M., Neuschwander-Tetri, B. A., Lavine, J. E., Tonascia, J., Unalp, A., et al. (2010). Pioglitazone, vitamin E, or placebo for nonalcoholic steatohepatitis. N Engl J Med 362, 1675-1685.
Sato, K., Gosho, M., Yamamoto, T., Kobayashi, Y., Ishii, N., Ohashi, T., Nakade, Y., Ito, K., Fukuzawa, Y., and Yoneda, M. (2015). Vitamin E has a beneficial effect on nonalcoholic fatty liver disease: a meta-analysis of randomized controlled trials. Nutrition 31, 923-930.
Schierwagen, R., Maybuchen, L., Zimmer, S., Hittatiya, K., Back, C., Klein, S., Uschner, F. E., Reul, W., Boor, P., Nickenig, G., et al. (2015). Seven weeks of Western diet in apolipoprotein-E-deficient mice induce metabolic syndrome and non-alcoholic steatohepatitis with liver fibrosis. Sci Rep 5, 12931.
Seki, E., De Minicis, S., Gwak, G. Y., Kluwe, J., Inokuchi, S., Bursill, C. A., Llovet, J. M., Brenner, D. A., and Schwabe, R. F. (2009). CCR1 and CCR5 promote hepatic fibrosis in mice. J Clin Invest 119, 1858-1870.
Shimamura, T., Fujisawa, T., Husain, S. R., Kioi, M., Nakajima, A., and Puri, R. K. (2008). Novel role of IL-13 in fibrosis induced by nonalcoholic steatohepatitis and its amelioration by IL-13R-directed cytotoxin in a rat model. J Immunol 181, 4656-4665.
Singh, S., Allen, A. M., Wang, Z., Prokop, L. J., Murad, M. H., and Loomba, R. (2015). Fibrosis progression in nonalcoholic fatty liver vs nonalcoholic steatohepatitis: a systematic review and meta-analysis of paired-biopsy studies. Clin Gastroenterol Hepatol 13, 643-654 e641-649; quiz e639-640.
Subramanian, S., Goodspeed, L., Wang, S., Kim, J., Zeng, L., Ioannou, G. N., Haigh, W. G., Yeh, M. M., Kowdley, K. V., O'Brien, K. D., et al. (2011). Dietary cholesterol exacerbates hepatic steatosis and inflammation in obese LDL receptor-deficient mice. J Lipid Res 52, 1626-1635.
Syn, W. K., Choi, S. S., Liaskou, E., Karaca, G. F., Agboola, K. M., Oo, Y. H., Mi, Z., Pereira, T. A., Zdanowicz, M., Malladi, P., et al. (2011). Osteopontin is induced by hedgehog pathway activation and promotes fibrosis progression in nonalcoholic steatohepatitis. Hepatology 53, 106-115.
Syn, W. K., Oo, Y. H., Pereira, T. A., Karaca, G. F., Jung, Y., Omenetti, A., Witek, R. P., Choi, S. S., Guy, C. D., Fearing, C. M., et al. (2010). Accumulation of natural killer T cells in progressive nonalcoholic fatty liver disease. Hepatology 51, 1998-2007.
Teratani, T., Tomita, K., Suzuki, T., Oshikawa, T., Yokoyama, H., Shimamura, K., Tominaga, S., Hiroi, S., Irie, R., Okada, Y., et al. (2012). A high-cholesterol diet exacerbates liver fibrosis in mice via accumulation of free cholesterol in hepatic stellate cells. Gastroenterology 142, 152-164 e110.
Van Rooyen, D. M., Larter, C. Z., Haigh, W. G., Yeh, M. M., Ioannou, G., Kuver, R., Lee, S. P., Teoh, N.C., and Farrell, G. C. (2011). Hepatic free cholesterol accumulates in obese, diabetic mice and causes nonalcoholic steatohepatitis. Gastroenterology 141, 1393-1403, 1403 e1391-1395.
White, D. L., Kanwal, F., and El-Serag, H. B. (2012). Association between nonalcoholic fatty liver disease and risk for hepatocellular cancer, based on systematic review. Clin Gastroenterol Hepatol 10, 1342-1359 e1342.
Wouters, K., van Gorp, P. J., Bieghs, V., Gijbels, M. J., Duimel, H., Lutjohann, D., Kerksiek, A., van Kruchten, R., Maeda, N., Staels, B., et al. (2008). Dietary cholesterol, rather than liver steatosis, leads to hepatic inflammation in hyperlipidemic mouse models of nonalcoholic steatohepatitis. Hepatology 48, 474-486.
Yamada, K., Mizukoshi, E., Sunagozaka, H., Arai, K., Yamashita, T., Takeshita, Y., Misu, H., Takamura, T., Kitamura, S., Zen, Y., et al. (2015). Characteristics of hepatic fatty acid compositions in patients with nonalcoholic steatohepatitis. Liver Int 35, 582-590.
Younossi, Z. M., Page, S., Rafiq, N., Birerdinc, A., Stepanova, M., Hossain, N., Afendy, A., Younoszai, Z., Goodman, Z., and Baranova, A. (2011). A biomarker panel for non-alcoholic steatohepatitis (NASH) and NASH-related fibrosis. Obes Surg 21, 431-439.
Yu, F. X., Zhao, B., and Guan, K. L. (2015). Hippo Pathway in Organ Size Control, Tissue Homeostasis, and Cancer. Cell 163, 811-828.
Zanconato, F., Forcato, M., Battilana, G., Azzolin, L., Quaranta, E., Bodega, B., Rosato, A., Bicciato, S., Cordenonsi, M., and Piccolo, S. (2015). Genome-wide association between YAP/TAZ/TEAD and AP-1 at enhancers drives oncogenic growth. Nat Cell Biol 17, 1218-1227.
Zhang H, et al., 2009 TEAD transcription factors mediate the function of TAZ in cell growth and epithelial-mesenchymal transition. J Biol Chem. 284(20):13355-62.
Zhao, B., Li, L., Lei, Q., and Guan, K. L. (2010). The Hippo-YAP pathway in organ size control and tumorigenesis: an updated version. Genes Dev 24, 862-874.
Zhao, B., Ye, X., Yu, J., Li, L., Li, W., Li, S., Yu, J., Lin, J. D., Wang, C. Y., Chinnaiyan, A. M., et al. (2008). TEAD mediates YAP-dependent gene induction and growth control. Genes Dev 22, 1962-1971.
Zuckerman, J. E. and Davis, M. E. (2015). Clinical experiences with systemically administered siRNA-based therapeutics in cancer. Nature Reviews Drug Discovery, 14:843-856.

### Example 3

Nonalcoholic fatty liver disease (NAFLD) begins with hepatosteatosis (fatty liver). While this is a relatively benign condition, about 20-30% will progress to non-alcoholic steatohepatitis (NASH)¹⁻⁹. This progression is caused by multiple “hits” acting on steatotic hepatocytes that cause liver inflammation, hepatocellular death, and, most importantly, liver fibrosis, which correlates best with clinical outcome in NASH¹⁰⁻¹². Identifying fibrosis in NAFLD is essential to predict liver-related outcomes and inform treatment decisions¹³. A blood signature of fibrosis could serve as a valuable, non-invasive diagnostic tool¹³⁻¹⁵.

Hepatocyte (HC) TAZ and Ihh are induced in human and mouse fibrotic NASH. TAZ causes progression to NASH fibrosis by inducing the synthesis and secretion of Indian hedgehog (Ihh).

Here, we validated blood IHH (Indian hedgehog) as a biomarker for NASH prediction. Specifically, we studied whether IHH can be a non-invasive plasma biomarker of fibrotic NASH, and whether it can be used as a non-invasive biomarker of therapeutic targeting of the TAZ and IHH for NASH, e.g., by HC-directed siRNAs.

### Results and Discussion

We have demonstrated that IHH was upregulated in NASH fibrosis livers through TAZ (WWTR1), a transcriptional factor. TAZ promotes NASH by inducing IHH, secreted by hepatocytes and activates hepatic stellate cells to cause liver fibrosis¹⁶⁻¹⁸. The therapeutic potential was illustrated by our showing that silencing hepatocyte-TAZ in experimental NASH using GalNAc-siTaz, a platform in use in humans¹⁹, reversed early NASH fibrosis¹⁸. In the livers of NASH patients and fibrotic mouse livers, IHH is induced significantly. In the siTAZ treated mice, IHH decreased in the liver tissues.

We have proven that IHH promotes NASH fibrosis under TAZ regulation in the liver. IHH can be secreted by hepatocytes; whether circulating IHH correlates with NASH fibrosis was unknown. To answer this question, we designed the experiment to quantify the IHH in circulation.

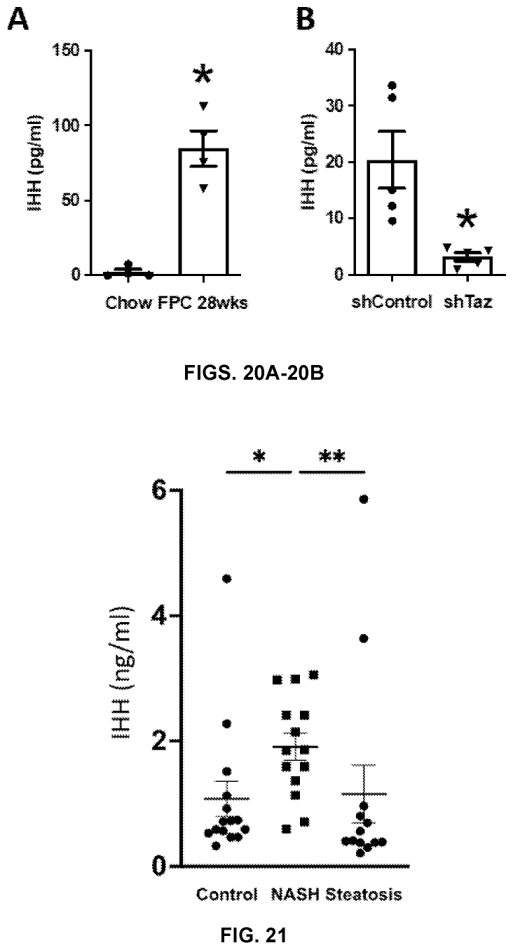

We used a well-established model of fibrotic NASH by feeding mice a diet rich in fructose, palmitate, and cholesterol (FPC) for 28 weeks. In this model, TAZ was induced in HCs after 10 weeks of feeding, similar to what occurs in the livers of humans with fibrotic NASH. First, we measured plasma IHH in 28 weeks mouse FPC model and found that plasma IHH increased about 40 folds in the NASH group compared with chow-fed non-NASH mice (FIG. 20A and Table 8).

TABLE 8

| | Plasma IHH level (pg/ml) | |
|---|---|---|
| Sample No. | Chow | FPC (28 weeks) |
| 1 | 0.223793 | 112.7325 |
| 2 | 1.270828 | 93.42895 |
| 3 | 7.419779 | 57.65842 |
| 4 | 0.147645 | 75.38187 |
| Mean | 2.266 | 84.8 |
| SEM (Standard error of the mean) | 1.737 | 11.83 |
| S.D. (standard deviation) | 3.474 | 23.66 |

We then determined whether plasma IHH reflected the decrease in HC-TAZ and liver IHH that occurs when HC-TAZ is silenced with HC-directed TAZ shRNA. For this purpose, mice were treated with AAV8-H1-shScrambled (Scr; control) or shTaz and then fed the FPC diet for 16 wks. This treatment lowers HC-TAZ and liver Ihh, and it decreases fibrotic NASH. After TAZ silencing, plasma IHH was also down-regulated significantly (FIG. 20B and Table 9), similar to our previous discovery in the livers¹⁶⁻⁸.

TABLE 9

| | Plasma IHH level (pg/ml) | |
|---|---|---|
| Sample No. | shControl | shTaz |
| 1 | 31.50158 | 3.897934 |
| 2 | 33.69084 | 4.297711 |
| 3 | 12.25518 | 0.909125 |
| 4 | 9.57096 | 4.716525 |
| 5 | 15.07265 | 2.051345 |
| Mean | 20.42 | 3.175 |
| SEM | 5.059 | 0.7265 |
| S.D. | 11.31 | 1.625 |

Next, we tested IHH concentration in human plasma samples. The data indicated that the mean IHH concentration was about 2 times higher in the NASH group than steatosis and obesity control plasma (FIG. 21 and Table 10).

TABLE 10

| | Plasma IHH level (ng/ml) | | |
|---|---|---|---|
| Sample No. | Control | NASH | Steatosis |
| 1 | 0.592257 | 0.711731 | 0.693899 |
| 2 | 0.567292 | 1.137916 | 0.387189 |
| 3 | 0.330126 | 0.597606 | 0.561942 |
| 4 | 0.718864 | 2.41647 | 0.403238 |
| 5 | 0.740262 | 2.992444 | 0.383622 |
| 6 | 2.280947 | 2.145424 | 0.380056 |
| 7 | 1.130783 | 1.851196 | 0.305162 |
| 8 | 0.590474 | 3.056639 | 0.212435 |
| 9 | 4.591974 | 1.596198 | 5.863396 |
| 10 | 1.514171 | 2.41647 | 0.802674 |
| 11 | 0.472782 | 2.978178 | 0.966729 |
| 12 | 0.922149 | 1.596198 | 3.639746 |
| 13 | 0.531628 | 1.861895 | 0.412154 |
| 14 | 0.469216 | 1.366166 | |
| 15 | 0.729563 | | |
| Mean | 1.079 | 1.909 | 1.155 |
| SEM | 0.2821 | 0.2155 | 0.464 |
| S.D. | 1.092 | 0.8062 | 1.673 |

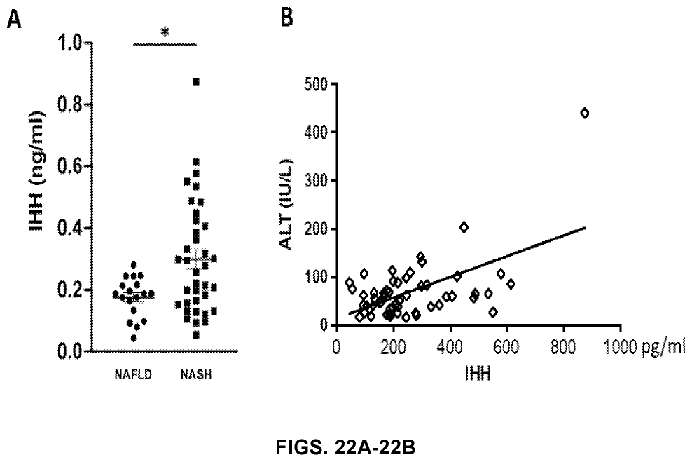

Further, the second cohort of human serum was conducted for validation. The results confirmed the discovery in the 1^{st} cohort plasma samples. IHH concentration increased in NASH vs. NAFLD serum (FIG. 22A and Table 11).

TABLE 11

| | Plasma IHH level (ng/ml) | |
|---|---|---|
| Sample No. | NAFLD | NASH |
| 1 | 0.2444042 | 0.0553754 |
| 2 | 0.2135233 | 0.150583 |
| 3 | 0.1750867 | 0.127223 |
| 4 | 0.1877372 | 0.3004128 |
| 5 | 0.097763 | 0.0937426 |
| 6 | 0.1642732 | 0.3167717 |
| 7 | 0.044042 | 0.5509262 |
| 8 | 0.2457906 | 0.2205937 |
| 9 | 0.0792553 | 0.1987933 |
| 10 | 0.1873906 | 0.4486482 |
| 11 | 0.1332536 | 0.1049374 |
| 12 | 0.2165386 | 0.2079432 |
| 13 | 0.1960206 | 0.1840633 |
| 14 | 0.2447161 | 0.2148056 |
| 15 | 0.1758492 | 0.1207418 |
| 16 | 0.0923216 | 0.2955605 |
| 17 | 0.1874945 | 0.3316403 |
| 18 | 0.2809692 | 0.1557471 |
| 19 | | 0.1965751 |
| 20 | | 0.4236593 |
| 21 | | 0.3861585 |
| 22 | | 0.1657289 |
| 23 | | 0.874431 |
| 24 | | 0.1312781 |
| 25 | | 0.2782658 |
| 26 | | 0.5780293 |
| 27 | | 0.0959608 |
| 28 | | 0.4881592 |
| 29 | | 0.5345326 |
| 30 | | 0.3613082 |
| 31 | | 0.6141438 |
| 32 | | 0.4829257 |
| 33 | | 0.1310701 |
| 34 | | 0.2574706 |
| 35 | | 0.4061219 |
| 36 | | 0.2987491 |
| Mean | 0.1759 | 0.2995 |
| SEM | 0.01523 | 0.03054 |
| S.D. | 0.06463 | 0.1832 |

Based on these two cohorts, using a plasma IHH threshold of 25% increase compared with the mean of the non-NASH population would lead to a sensitivity of approximately 60% and a specificity of approximately 80%, which is comparable to other NASH biomarkers in the literature (Corey et al., ADAMTSL2 protein and a soluble biomarker signature identify significant and advanced fibrosis in adults with NAFLD, Journal of Hepatology (2021), DOI: https://doi.org/10.1016/j.jhep.2021.09.026).

Moreover, the regression analysis indicated that IHH concentration in the serum had a positive correlation with the patient ALT level (FIG. 22B and Table 12), which is a liver injury marker.

TABLE 12

| Sample No. | Plasma IHH (pg/ml) | ALT (IU/L) |
|---|---|---|
| 1 | 244.4042 | 16 |
| 2 | 213.5233 | 25 |
| 3 | 175.0867 | 72 |
| 4 | 187.7372 | 18 |
| 5 | 97.76304 | 26 |
| 6 | 164.2732 | 58 |
| 7 | 44.04198 | 88 |
| 8 | 245.7906 | 62 |
| 9 | 79.25527 | 17 |
| 10 | 187.3906 | 34 |
| 11 | 133.2536 | 55 |
| 12 | 216.5386 | 39 |
| 13 | 196.0206 | 113 |
| 14 | 244.7161 | 98 |
| 15 | 175.8492 | 22 |
| 16 | 92.32162 | 41 |
| 17 | 187.4945 | 23 |
| 18 | 280.9692 | 20 |
| 19 | 55.37539 | 75 |
| 20 | 150.583 | 46 |
| 21 | 127.223 | 40 |
| 22 | 300.4128 | 132 |
| 23 | 93.74263 | 62 |
| 24 | 316.7717 | 83 |
| 25 | 550.9262 | 27 |
| 26 | 220.5937 | 50 |
| 27 | 198.7933 | 91 |
| 28 | 448.6482 | 203 |
| 29 | 104.9374 | 40 |
| 30 | 207.9432 | 43 |
| 31 | 184.0633 | 68 |
| 32 | 214.8056 | 88 |
| 33 | 120.7418 | 18 |
| 34 | 295.5605 | 142 |
| 35 | 331.6403 | 38 |
| 36 | 155.7471 | 48 |
| 37 | 196.5751 | 37 |
| 38 | 423.6593 | 101 |
| 39 | 386.1585 | 59 |
| 40 | 165.7289 | 67 |
| 41 | 874.431 | 440 |
| 42 | 131.2781 | 67 |
| 43 | 278.2658 | 25 |
| 44 | 578.0293 | 107 |
| 45 | 95.96079 | 107 |
| 46 | 488.1592 | 65 |
| 47 | 534.5326 | 65 |
| 48 | 361.3082 | 42 |
| 49 | 614.1438 | 85 |
| 50 | 482.9257 | 57 |
| 51 | 131.0701 | 67 |
| 52 | 257.4706 | 109 |
| 53 | 406.1219 | 60 |
| 54 | 298.7491 | 81 |

The data showed that high IHH can be a biomarker for NASH fibrosis prediction and diagnosis, and both plasma and serum samples are suitable for the ELISA assay. In our previous TAZ-IHH study, we found that TAZ is a key transition molecule from NAFLD to NASH and IHH starts response after TAZ increases and before fibrosis builds up¹⁶. IHH has an advantage on early prediction compared to other fibrosis-based noninvasive diagnosis markers. As the discovery based on the detailed mechanism in the animal model, IHH is associated with NASH fibrosis, and the data support the use of IHH as a tool to identify NASH patients in the NAFLD population.

In summary, we measured plasma and serum samples from NAFLD patients and mouse NASH models and have established the association between NASH fibrosis and blood IHH concentration.

### Materials and Methods

### Mouse Plasma

Male wild-type mice C57BL/6J (#000664, 12 weeks old) were obtained from Jackson Laboratory and were allowed to adapt to housing in the Columbia University Medical Center Institute of Comparative Medicine for 1 week prior to random assignment to experimental cohorts. The mice were then fed the FPC diet (Envigo, TD.160785 PWD) for 28 weeks (FIG. 20A) or 16 weeks (FIG. 20B). Chow diet (Picolab rodent diet 20, #5053) fed mice served as a control. AAV8-H1 shTaz virus or control AAV8-H1 virus (Packaged by Vector Biolabs, 2×10¹¹ genome copies/mouse) was delivered by tail vein injection after 8 weeks of the diet. After the mice were sacrificed, the plasma IHH was measured by ELISA kit (Biomatik, Cat #: EKU04700). Animals were housed in standard cages at approximately 22° C. in a 12 hr light/12 hr dark cycle. All animal experiments were performed in accordance with institutional guidelines and regulations and approved by the Institutional Animal Care and Use Committee at Columbia University.

### Human Plasma and Serum

Human plasma and serum from 96 subjects were included. Cases with NAFLD activity score of 1-3 were classified as early NAFLD (no fibrosis), while cases with NAS>=4 and fibrosis stage 1-4 were classified as NASH. Cases with steatosis score>=1 and inflammation and ballooning scores of 0 and no fibrosis were classified as steatosis. Cases with NAS=0 were classified as normal control. Patients gave informed consent at the time of recruitment, and their records were anonymized and de-identified. Studies were approved by the Columbia University Institutional Review Board and by the Partners Human Research Committee and conducted in accordance with National Institutes of Health and institutional guidelines for human subject research. The plasma IHH was measured by ELISA kit (Biomatik, Cat #: EKU04699). ALT was measured by endpoint method (TECO Diagnostics, Cat #: A526-120).

### ELISA Assay

The standards were prepared in serial dilutions (1,000 μg/mL, 500 μg/mL, 250 μg/mL, 125 μg/mL, 62.5 μg/mL, 31.25 μg/mL, 15.625 μg/mL, 0 μg/mL). 50 μL of dilution was added to each of the standards, blanks and samples in the appropriate wells in the plate. 50 μL of detection antibody was then added to each well immediately. The plate was shaken gently and covered with a plate sealer. The plate was then incubated for 1 hour at 37° C. The solution was aspirated. 350 μL of 1× Wash Solution was added to each well using a multi-channel pipette to wash. The wash was repeated 3 times. 100 μL of Detection Reagent HRP working solution was added to each well. The plate was covered with the plate sealer and incubated for 30 minutes at 37° C. The aspiration/wash process was repeated for a total of 5 times. 90 μL of Substrate Solution was added to each well. The plate was covered with the plate sealer and incubated for 10-20 minutes at 37° C. 50 μL of Stop Solution was added to each well. The microplate reader was run, and measurement taken at 450 nm immediately.

### ALT Measurement

The standards were prepared in serial dilutions (210 IU/L, 140 IU/L, 70 IU/L, 35 IU/L, 17.5 IU/L, 0 IU/L). 50 μl of ALT (SGPT) substrate was transferred to a 96-well plate, and placed in a 37° C. heating bath for 5 mins. At timed intervals (about 13-30 secs), 10 μl standard, blank and serum samples were added. The reactions were mixed and returned to 37° C. heating bath for 30 mins. 50 μl of ALT (SGPT) color reagent was added to each well, maintaining the timed interval sequence. The reactions were mixed and returned to 37° C. heating bath for 10 mins. After 10 mins, 200 μl of ALT (SGPT) color developer (maintaining the same timed intervals) was added. The reactions were mixed and returned to 37° C. heating bath for 5 mins.

The spectrophotometer was zeroed with the reagent “blank” at 505 nm. Absorbance of all wells was read and recorded (wavelength range: 500-520 nm).

### REFERENCES

1 Loomba, R. & Sanyal, A. J. The global NAFLD epidemic. Nat Rev Gastroenterol Hepatol 10, 686-690, doi:10.1038/nrgastro.2013.171 (2013).
2 Corey, K. E. & Kaplan, L. M. Obesity and liver disease: the epidemic of the twenty-first century. Clin. Liver Dis 18, 1-18 (2014).
3 Wree, A., Broderick, L., Canbay, A., Hoffman, H. M. & Feldstein, A. E. From NAFLD to NASH to cirrhosis-new insights into disease mechanisms. Nat Rev Gastroenterol Hepatol 10, 627-636, doi:10.1038/nrgastro.2013.149 (2013).
4 Day, C. P. & James, O. F. Steatohepatitis: a tale of two “hits”? Gastroenterology 114, 842-845 (1998).
5 Tilg, H. & Moschen, A. R. Evolution of inflammation in nonalcoholic fatty liver disease: the multiple parallel hits hypothesis. Hepatology 52, 1836-1846 (2010).
6 Takaki, A., Kawai, D. & Yamamoto, K. Multiple hits, including oxidative stress, as pathogenesis and treatment target in non-alcoholic steatohepatitis (NASH). Int. J. Mol. Sci 14, 20704-20728 (2013).
7 Larrain, S. & Rinella, M. E. A myriad of pathways to NASH. Clin. Liver Dis 16, 525-548 (2012).
8 Bohinc, B. N. & Diehl, A. M. Mechanisms of disease progression in NASH: new paradigms. Clin. Liver Dis 16, 549-565 (2012).
9 Pacana, T. & Sanyal, A. J. Recent advances in understanding/management of non-alcoholic steatohepatitis. F1000Prime. Rep 7, 28 (2015).
10 Angulo, P. et al. Liver fibrosis, but no other histologic features, is associated with long-term outcomes of patients with nonalcoholic fatty liver disease. Gastroenterology 149, 389-397 (2015).
11 Dulai, P. S. et al. Increased risk of mortality by fibrosis stage in nonalcoholic fatty liver disease: Systematic review and meta-analysis. Hepatology 65, 1557-1565, doi:10.1002/hep.29085 (2017).
12 Vilar-Gomez, E. et al. Fibrosis severity as a determinant of cause-specific mortality in patients with advanced nonalcoholic fatty liver disease: a multi-national cohort study. Gastroenterology 155, 443-457.e417, doi:10.1053/j.gastro.2018.04.034 (2018).
13 Corey, K. E. et al. ADAMTSL2 protein and a soluble biomarker signature identify significant and advanced fibrosis in adults with NAFLD. J Hepatol, doi:10.1016/j.jhep.2021.09.026 (2021).
14 Nielsen, M. J. et al. Comparison of ADAPT, FIB4 and APRI as non-invasive predictors of liver fibrosis and NASH within the CENTAUR Screening Population. J Hepatol, doi:10.1016/j.jhep.2021.08.016 (2021).
15 Luo, Y. et al. SOMAscan Proteomics Identifies Serum Biomarkers Associated With Liver Fibrosis in Patients With NASH. Hepatol Commun 5, 760-773, doi:10.1002/hep4.1670 (2021).
16 Wang, X. et al. Hepatocyte TAZ/WWTR1 promotes inflammation and fibrosis in nonalcoholic steatohepatitis. Cell Metab 24, 848-862 (2016).
17 Wang, X. et al. Cholesterol stabilizes TAZ in hepatocytes to promote experimental non-alcoholic steatohepatitis. Cell Metab 31, 969-986.e967, doi:10.1016/j.cmet.2020.03.010 (2020).
18 Wang, X. et al. A therapeutic silencing RNA targeting hepatocyte TAZ prevents and reverses fibrosis in nonalcoholic steatohepatitis in mice. Hepatol Commun 3, 1221-1234, doi:10.1002/hep4.1405 (2019).
19 Springer, A. D. & Dowdy, S. F. GalNAc-siRNA conjugates: leading the way for delivery of RNAi therapeutics. Nucleic Acid Ther 28, 109-118, doi:10.1089/nat.2018.0736 (2018).

The scope of the present invention is not limited by what has been specifically shown and described hereinabove. Those skilled in the art will recognize that there are suitable alternatives to the depicted examples of materials, configurations, constructions and dimensions. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description is offered by way of illustration only and not as a limitation.

1. A method for diagnosing a liver condition or susceptibility to liver condition in a subject, wherein the liver condition is selected from the group consisting of a fatty liver disease, a non-alcoholic fatty liver disease, adiposity, and combinations thereof, the method comprising:
(a) obtaining a sample from the subject;
(b) determining a level of Indian hedgehog (IHH) in the sample;
(c) comparing the level of IHH in the sample with a level of IHH in a control sample; and
(d) diagnosing that the subject has the liver condition or diagnosing the subject as being susceptible to the liver condition, if the level of IHH in the sample increases by at least 25% compared to its level in the control sample.

2. A method of treating a subject with a liver condition or susceptible to a liver condition, wherein the liver condition is selected from the group consisting of a fatty liver disease, a non-alcoholic fatty liver disease, adiposity, and combinations thereof, the method comprising:
(a) obtaining a sample from the subject;
(b) determining a level of IHH in the sample;
(c) comparing the level of IHH in the sample with a level of IHH in a control sample; and
(d) treating the subject, if the level of IHH in the sample increases by at least 25% compared to its level in the control sample.

3. The method claim 2, wherein the non-alcoholic fatty liver disease is non-alcoholic steatosis hepatis or non-alcoholic steatohepatitis (NASH).

4. The method claim 2, wherein the fatty liver disease is steatosis hepatis or steatohepatitis.

5. The method of claim 2, wherein the sample is a plasma, serum or blood sample.

6. The method claim 2, wherein the increase in the level of IHH is at least 50%.

7. The method claim 2, wherein the increase in the level of IHH is at least 60%.

8. The method of claim 2, wherein the increase in the level of IHH is at least 70%.

9. The method of claim 2, wherein the level of IHH is determined by enzyme-linked immunosorbent assay (ELISA).

10. The method claim 2, wherein the level of IHH is determined by mass spectrometry (MS).

11. The method of claim 2, wherein in step (d) a therapeutically effective amount of an inhibitor of TAZ is administered to the subject.

12. The method of claim 11, wherein the inhibitor of TAZ is selected from the group consisting of proteins, nucleic acids, and combinations thereof.

13. The method of claim 12, wherein the nucleic acid is selected from the group consisting of an antisense oligonucleotide, siRNA, shRNA, and combinations thereof.

14. The method of claim 12, wherein the nucleic acid comprises nucleic acid sequence selected from the group consisting of: SEQ ID NO:1, SEQ ID NO:2, SEQ ID NO:55-SEQ ID NO:72, and SEQ ID NO:81.

15. The method of claim 2, wherein in step (d) a therapeutically effective amount of one or more of the following is administered to the subject: an inhibitor of IHH, an inhibitor of YAP, an inhibitor of TEAD1, and inhibitor of TEAD2, an inhibitor of TEAD3, and an inhibitor of TEAD4.

16. The method of claim 2, wherein in step (d) a therapeutically effective amount of one or more of the following is administered to the subject: a therapeutic agent for treatment of steatosis hepatis, a therapeutic agent for treatment of steatohepatitis, a therapeutic agent for treatment of non-alcoholic fatty liver disease, a therapeutic agent for treatment of non-alcoholic steatohepatitis, a therapeutic agent for treatment of adiposity and combinations thereof.

17. The method of claim 2, wherein in step (d) a therapeutically effective amount of one or more of the following is administered to the subject: an antidiabetic drug, and an insulin sensitizer.

18. The method of claim 2, wherein in step (d) a therapeutically effective amount of one or more of the following is administered to the subject: rosiglitazone; pioglitazone; losartan; simtuzumab; GR-MD-02; and obeticholic acid (OCA).

19. The method of claim 2, wherein the control sample is from a healthy subject or a plurality of healthy subjects.

20. The method of claim 2, wherein the subject is human.

21. (canceled)
